(19) United States
(12) Patent Application Publication
(10) Pub. No.: US 2022/0409650 A1
(43) Pub. Date: Dec. 29, 2022

(54) COMPOSITIONS AND METHODS FOR ASCAROSIDE MODIFICATION OF MAMMALIAN MICROBIOTA

(71) Applicants: Holoclara, Inc., Los Angeles, CA (US); California Institute Of Technology, Pasadena, CA (US)

(72) Inventors: Andrea Choe, Pasadena, CA (US); Brian Stoltz, Pasadena, CA (US); Simon Bailey, Pasadena, CA (US); Eduardo Martinez, Pasadena, CA (US)

(21) Appl. No.: 17/616,506

(22) PCT Filed: Jun. 3, 2020
(86) PCT No.: PCT/US20/35900

§ 371 Date: Dec. 3, 2021

Related U.S. Application Data
(60) Provisional application No. 62/856,619, filed on Jun. 3, 2019.

(51) Int. Cl. A61K 31/7028; A61P 1/14; A61K 9/00

(52) U.S. Cl. CPC A61K 31/7028; A61P 1/14; A61K 9/0053

(57) ABSTRACT

Described herein are composition of ascarosides, methods of using them, and the like for inhibiting bacterial population growth or for altering the relative ratio of sub-populations of first and second bacteria in a mixed population of bacteria. The invention is particularly useful, for example, for modifying a microbiome of a patient (e.g., for treatment of gut microbiome dysfunction).

### CROSS REFERENCE TO RELATED APPLICATIONS

This patent application claims priority to U.S. provisional patent application No. 62/856,619, filed on Jun. 3, 2019, titled “COMPOSITIONS AND METHODS FOR ASCAROSIDE MODIFICATION OF MAMMALIAN MICROBIOTA”, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are compositions and methods of use of one or more ascaroside “ascr” pheromones or ascaroside derivative pheromones (including one or a combination of such pheromones) to modify a mammalian microbiome.

### COMMON OWNERSHIP UNDER JOINT RESEARCH AGREEMENT 35 U.S.C. 102(c)

The subject matter disclosed in this application was developed, and the claimed invention was made by, or on behalf of, one or more parties to a joint Research Agreement that was in effect on or before the effective filing date of the claimed invention. The parties to the Joint Research Agreement are as follows: California Institute of Technology and Holoclara, Inc.

### BACKGROUND

Ascaroside pheromones were first identified in the roundworm C. elegans, and were known to mediate their mating behavior. Many roundworm species secret these pheromones, despite the fact that these species are otherwise biologically diverse, and occupy very diverse niches such as soil, mammal, plant, insect. Many roundworms species utilize these pheromones to mediate distinct behaviors such as mating, aggregation, hibernation, etc. Different roundworm species also use different combinations of pheromones to communicate.

FIG. 1 is a chart illustrating ascaroside pheromones produced by a diverse range of species of roundworms. The ascaroside pheromone language resembles that used by bacteria during quorum sensing.

Ascaroside pheromones have been proposed for the treatment of autoimmune or inflammatory disorders. For example, PCT/US2012/050031, U.S. Pat. No. 9,868,754 (describing the use of isolated (Ascr #7) or a salt thereof to treat asthma, inflammatory bowel disease, or type one diabetes), U.S. Pat. Nos. 9,534,008, 10,183,963 (describing a method of reducing eosinophils and/or lymphocytes using Ascr #7 or a salt thereof), U.S. Patent Application No. US20190053451, and CN Patent No. 104105489 (describing Ascr #1, Ascr #3, Ascr #7, Ascr #8, Ascr #9 for asthma treatment). Each of these patents and patent applications is herein incorporated by reference in its entirety.

However, to date, the use of Ascaroside pheromones has not been suggested for the treatment of mammalian microbiomes. Described herein are compositions of Ascaroside pheromones and methods of using Ascaroside pheromones to modify a mammalian microbiome (e.g., gut microbiome, vaginal microbiome, etc.).

### SUMMARY OF THE DISCLOSURE

Humans have coevolved with their microbes over thousands of years, but this relationship is now being dramatically affected by shifts in the collective human microbiome resulting from changes in the environment and societal norms, and especially in the last fifty years, due to the prevalence of antibiotics in our environment. Resulting perturbations of intestinal host-microbe interactions have enhanced the spread of so-called “western” disorders.

Described herein are compositions and methods that may be used to adjust or modify a patient's microbiome (e.g., a composition of ascaroside for increasing the diversity of a subject's gut microbiota). In particular, described herein as compositions including one or more ascarosides. These compositions may include an ascaroside having the formulation of Formula I:

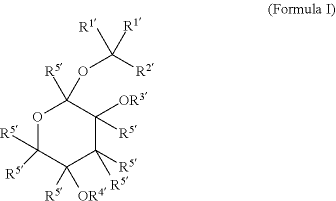

or a pharmaceutical equivalent, derivative, analog, and/or salt thereof. As readily apparent to one of skill in the art, the compound may be further defined by various R groups.

In some variations, the composition, and method of using the composition to adjust or modify a patient's microbiome (e.g., for increasing the diversity of a subject's gut microbiota and/or for treating a disorder such as constipation) may be a compositions of one or more Ascaroside having the formula of Formula II (or a pharmaceutical equivalent, derivative, analog, and/or salt thereof):

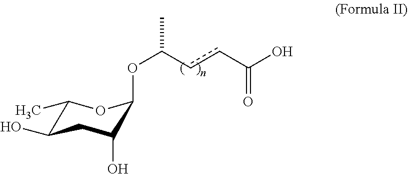

where: [symbol] represents a double or single bond, and ( )n represents (CH₂)ₙ where n may be between 1-12.

These compositions may include, for example, one or more ascaroside having the formula described above, in which the one or more ascarosides include one or more of:

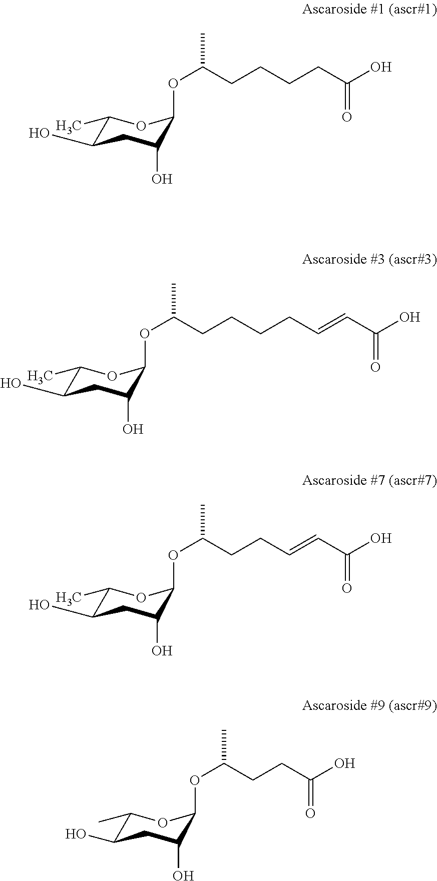

The Ascarosides having Formula II are a sub-set of the Ascarosides having the formula of Formula I.

In some variations, the composition, and method of using the composition to adjust or modify a patient's microbiome (e.g., for increasing the diversity of a subject's gut microbiota and/or for treating a disorder such as constipation) may be a compositions of one or more naturally-occurring Ascaroside (e.g., ascr #1, ascr #2, ascr #3, ascr #5, ascr #7, ascr #9, ascr #10, ascr #11, ascr #12, ascr #14, ascr #16, ascr #18, ascr #20, ascr #22, ascr #24, and ascr #26), or a pharmaceutical equivalent, derivative, analog, and/or salt thereof. The naturally-occurring Ascarosides are a subset of the Ascarosides having the formula of Formula I which overlaps with a subset of the Ascarosides having the Formula of Formula II (but also includes ascr #5).

In some variations, the composition, and method of using the composition to adjust or modify a patient's microbiome (e.g., for increasing the diversity of a subject's gut microbiota and/or for treating a disorder such as constipation) may be a compositions of one or more Ascaroside derivatives having the formula of Formula (III):

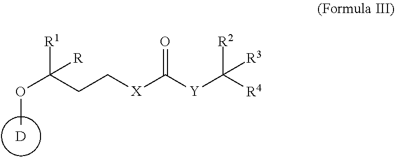

wherein Ring D is selected from:

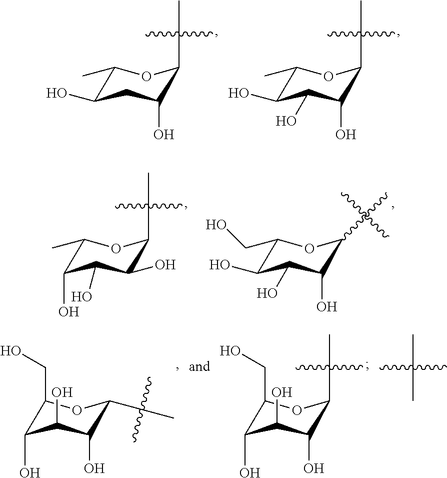

represents the point of attachment of Ring D to the oxygen atom;
X is —C(R⁵)₂—C(R⁵)₂— or E or Z —CH═CH—;

### Y is O or NR⁶;

R is H or CH₃, R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl; R⁵, independently for each occurrence, is H or OH, or two instances of R⁵, taken together with the carbon atom or carbon atoms to which they are bonded, form a 3-membered carbocyclic ring; and
R⁶ is H or C₁-C₆ alkyl, or, when Y is NR⁶, R⁴ and NR⁶, taken together with the carbon atom to which they are bonded, form a 5- or 6-membered heterocyclic ring.

Also described herein are methods (and compositions) for using any of these compositions to modify the patient's microbiome, e.g., to increase diversity of the gut microbiota. Also described are methods of (and compositions for) selectively altering a subject's gut microbiota by administering to the subject's gastrointestinal system a composition comprising pharmaceutically-acceptable carrier and an effective amount of ascaroside and/or ascaroside derivative thereby selectively modifying the composition of the gut microbiota to increase one or more of: the diversity of the gut microbiota, the amount of Bifidobacterium, the amount of Akkermansia and the amount of Adlercreutzia within the subject's gastrointestinal system. Also described herein are methods of (and compositions for) treating constipation in a subject comprising administering to the subject a composition of ascaroside and/or ascaroside derivative to increase fecal softness.

### BRIEF DESCRIPTION OF THE DRAWINGS

FIG. 1 is a graph illustrating Ascarosides produced by a wide range of nematode species. The plot shown summarizes the results from HPLC-MS-based screens of worm exudate samples. Colors represent relative abundance of ascarosides detected by HLPC-MS, as indicated in the bar diagram on the right. The phylogenic tree was constructed by comparison of small subunit ribosomal DNA (SSU rDNA) sequences obtained from GenBank. This graph is adapted from Choe et. al (2012).

FIG. 2A illustrates similarities of signaling molecules in nematodes and bacteria N-acyl homoserine lactones (AHLs). Also adapted from Choe et al. (2012). AHLs are a family of small molecules that mediate bacterial quorum sensing and are based on homoserine lactone and feature species-specific variations in the N-acyl chains. Although ascarosides and N-Acyl Homoserine lactones are structurally different molecules, they both play significant roles in mediating survival strategies of the organisms producing them. Because of their structural differences, it has not previously been suggested that the ascarosides may act directly or indirectly in the N-Acyl Homoserine lactone pathways of bacteria. FIG. 2A also illustrates Ascarosides having a dideoxysugar ascarylose (including a tetrahydropyran) as scaffold to which variable lipid chains are attached.

FIG. 2B illustrates naturally-occurring Ascarosides (ascrs #1, #2, #3, #5, #7, #9, #10, #11, #12, #14, #16, #18, #20, #22, #24, and #26) that are secreted by free living or parasitic worms.

FIG. 2C illustrates the Formula II sub-group of ascarosides (indicating the sub-set of naturally-occurring ascarosides.

FIGS. 2D-2G illustrate examples of ascarosides as described herein.

FIG. 3 illustrates one example of a graph showing the increased alpha diversity of the gut microbiota following treatment with an ascaroside as described herein. In FIG. 3, alpha diversity was calculated from taxonomic profiles using Shannon's diversity index. Mice from Experiment 3, treated with Ascr #7, were examined, with stools collected at the end of Experiment 3 and compared between the control and treatment.

FIG. 4 illustrates another example of a graph showing the increase in alpha diversity of the gut microbiota following treatment with an ascaroside. In FIG. 4, alpha diversity was calculated from taxonomic profiles using Shannon's diversity index. Mice from Experiment 1, treated with Ascr #9, were examined, with stools collected at the beginning and end of Experiment 1 and compared between the control and treatment. The data shown here represents data compared at the end of the experiment and compared between the control and treatment.

FIG. 5 is one example of a graph showing Beta Diversity in treated mice gut microbiota. In FIG. 5, pairwise differences in taxonomic profiles were examined. The dot plot in FIG. 5 is an ordination representing differences in community composition (or Beta Diversity) estimated with Bray-Curtis dissimilarities. Permanova is used to formally test whether these differences are significant. Like regular (M)ANOVAs, Permanovas can be used to estimate differences in composition between pairs of treatment levels, where here the data is shown to be significant. Mice from Experiment 3 were studied, with stools collected at the end of Experiment 3 and compared between the control and treatment.

FIG. 6 is a graph showing Beta Diversity in treated mice gut microbiota. As in FIG. 5, pairwise differences in taxonomic profiles were examined; dot plot shown an ordination representing differences in community composition (or Beta Diversity) estimated with Bray-Curtis dissimilarities. Permanova is used to formally test whether these differences are significant. Like regular (M)ANOVAs, Permanovas can be used to estimate differences in composition between pairs of treatment levels, where here the data is shown to be significant. In FIG. 6, mice from Experiment 1 were studied, with stools collected at the beginning and end of Experiment 1 and compared between the control and treatment. The data shown here represents data compared at the end of the experiment and compared between the control and treatment.

FIG. 7 is a table illustrating statics calculated from FIG. 6.

FIG. 8 is one example of a graph showing an increase in Bifidobacter catenulatum bacteria following treatment with ascaroside. In FIG. 8, the ascaroside examined is Ascr #7, which shows a specific increase in Bifidobacter catenulatum bacteria in subject gut microbiota following 31 days of daily treatment. Metric multidimensional scaling analysis was used to visualize microbiome similarities. Permutational analysis of variance (PERMANOVA) was used to formally test for the significance of overall microbiome differences. Mice from Experiment 3 were studied, with stools collected at the end of Experiment 3 and compared between the control and treatment.

FIG. 9 is one example of a graph showing an increase in Bifidobacter, Akkermansia, and Adlercreutzia bacteria following treatment with ascaroside. In FIG. 9, the treatment with one or more ascaroside (e.g., Ascr #7), increases Bifidobacter, Akkermansia, and Adlercreutzia bacteria. Metric multidimensional scaling analysis was used to visualize microbiome similarities. Permutational analysis of variance (PERMANOVA) was used to formally test for the significance of overall microbiome differences. Mice from Experiment 1 were studied, with stools collected at the beginning and end of Experiment 1 and compared between the control and treatment. The data shown in FIG. 9 represents data compared at the end of the experiment and compared between the control and treatment.

FIG. 10 shows a comparison between stool consistency (e.g., in relation to constipation) for untreated mice (left, control) and mice treated with naturally-occurring ascaroside (center, treated with Ascr #7), or with ascaroside derivative (right, treated with Compound 1, see table 3), showing an overall increase the softness and smoothness of mouse feces following treatment. The mice treated with ascaroside (e.g., naturally-occurring ascaroside or ascaroside derivative) had a significantly higher proportion of feces with Type 4 stools, which is “smooth and soft” as compared to untreated mice. Mice from Experiment 3 were studied, with stools collected at the end of Experiment 3 and compared between the control and treatment.

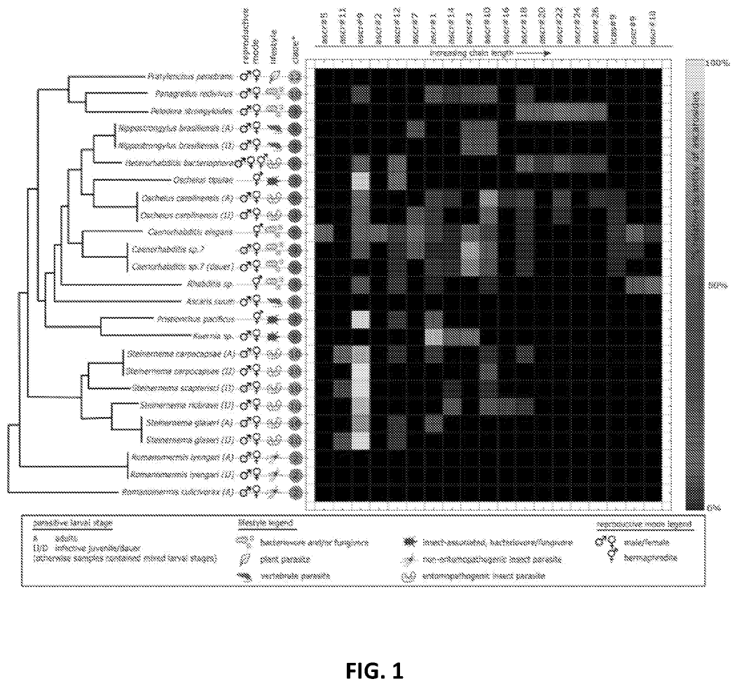

### DETAILED DESCRIPTION

In general, described herein are compositions, including ascaroside compositions, for the modification of mammalian microbiomes, and method of using ascaroside to modify mammalian microbiomes. These methods and compositions may be used to increase the diversity of gut microbiota. These methods and compositions may be used to treat constipation. These methods and compositions may be used to increase the absolute amount and/or proportion of one or more gut bacteria, such as one or more of Bifidobacterium, Akkermansia and Adlercreutzia. In particular, these compositions and methods may be used to restore a disrupted microbiome, e.g., to help reestablish a healthy microbiome, to protect an existing microbiome from disruption, and/or to modify an existing microbiome to help set new healthy microbiome. In some variations, the methods and compositions described herein may be used in conjunction with an antibiotic. These methods and compositions may be used as part of an animal feedstock and/or water source. These methods and compositions may be formulated for daily, weekly or monthly delivery by a human or non-human patient (e.g., mammalian).

The factors that influence gut colonization and the various factors leading to microbial imbalance or maladaptation (e.g., dysbacteriosis) remain poorly understood. One way of looking for potential contributing factors is to examine what local environmental factors have co-evolved with the mammalian gut microflora. The benefit of roundworms on human health has been recognized by studies where humans have consumed roundworms for therapeutic effect. However, consumption of roundworms is considered unpalatable and there have been some safety concerns, such as roundworm burrowing into the intestinal wall. Humans in western civilizations do not have worms residing in their gut. Further, the ingesting roundworms does is potentially dangerous and may be of limited effect.

There is a need in the art to develop roundworm-derived compounds, and combination of compounds, that are improved compared to whole roundworm administration, specifically towards the beneficial modification of the diversity and distribution of the gut microbiota.

Ascarosides are a family of small-molecule pheromones produced by roundworms that are known to mediate roundworm behavior (such as mate-finding, aggregation, and hibernation). These pheromones may induce responses from plants, mammals, and fungi. Given the broad evolutionary implications, it is possible that these pheromones also interact with gut bacteria. However, mammalian studies have not been performed in individuals with roundworms to identify the presence of ascarosides and its connection to differences in their microbiome, and (prior to the work described herein) it was unknown how ascarosides may modify the mammalian gut microbiome.

The term “ascaroside” refers to any of a group of glycolipids, containing the sugar ascarylose, found in most nematode worms. Ascarosides represent an evolutionarily conserved family of nematode-derived small molecules that serve essential functions in regulating development and social behaviors (Choe et al. (2012) Curr Biol., 22:772-780; Pungaliya et al. (2009) Proc Natl Acad Sci., 106:7708-7713; Srinivasan et al. (2008) Nature 454:1115-1118; Srinivasan et al. (2012) PLoS Biol 10:e1001237; von Reuss et al. (2012) J Am Chem Soc., 134:1817-1824; Butcher et al. (2007) Nat. Chem. Biol., 3:420-422; Golden et al. (1982) Science 218:578-580; Jeong et al. (2005) Nature 433:541-545; Kaplan et al. (2012) PLoS ONE 7:e38735; Ludewig et al. (2013) WormBook, 1-22; Noguez, J et al. (2012) ACS Chem Biol 7:961-966). Ascarosides are glycosides of the dideoxysugar ascarylose that carry a fatty acid-derived lipophilic side chain and have been identified exclusively from nematodes. For example, in the model organisms Caenorhabditis elegans, and Pristionchus pacificus as well as in the insect parasitic nematode Heterorhabditis bacteriophora, ascarosides regulate entry into stress resistant dispersal or infective larval stages. Whereas some nematode ascarosides (NAs) are broadly produced among different nematode species, other NAs are highly species-specific or are associated primarily with a specific ecology. For example, Ascr #9 is particularly common among entomopathogenic (insect-parasitic) nematodes, whereas the longer-chained Ascr #18 is produced by several species of the plant-parasitic genus Meloidogyne.

A compositions as disclosed herein may comprise between 0.1 and 95% by weight of ascaroside, e.g., between 0.5 and 90%, as the active ingredient; this percentage may represent the percentage of a single ascaroside or an aggregate of multiple types of ascarosdies.

The compositions may include substantially pure ascaroside. The term “substantially pure” refers to a preparation comprising at least 50-60% by weight of a given material (e.g., small molecule, nucleic acid, oligonucleotide, protein, etc.). In some variations the preparation may comprise at least 75% by weight, between about 90-95% by weight, etc. of the given compound (e.g., ascaroside). Purity may be measured by methods appropriate for the given compound (e.g. chromatographic methods, agarose or polyacrylamide gel electrophoresis, HPLC-MS analysis, and the like). In some variations the compositions may include 5% or more (e.g., 10% or more, 15% or more, 20% or more, 25% or more, 30% or more, etc.) of one or more ascaroside. This composition may be prepared for delivery as a drug (e.g., with one or more excipient, diluent, or carrier). In some variations the composition may be configured to time-release delivery form.

The term “functional” as used herein implies that the ascaroside is functional for the recited assay or purpose, e.g., for modulation of gut microbiome, treatment of constipation, etc.

The ascarosides for use in the methods and compositions described herein can vary in structure. The term “alkyl” refers to an aliphatic hydrocarbon group which may be a linear, branched, or cyclic hydrocarbon structure or combination thereof. Representative alkyl groups are those having 24 or fewer carbon atoms, for instance, methyl, ethyl, n-propyl, ipropyl, n-butyl, s-butyl, t-butyl, n-pentyl, i-pentyl, n-hexyl, and the like. Lower alkyl refers to alkyl groups having about 1 to about 6 carbon atoms in the chain. Branched alkyl means that one or more lower alkyl groups such as methyl, ethyl, or propyl are attached to a linear alkyl chain.

The statement that alkyl is intended to include linear, branched, or cyclic hydrocarbon structures and combinations thereof means that an “alkyl” group also includes the following combination of linear and cyclic structural elements

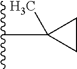 [SMILES: *C1(C)CC1]

(and similar combinations).

The term “alkenyl” as used herein, alone or in combination, refers to a straight-chain or branched-chain hydrocarbon radical having one or more double bonds optionally substituted and containing from 2 to 20, preferably 2 to 6, carbon atoms. Alkenyl refers to a carbon-carbon double bond system attached at two or more positions such as ethenylene [(—CH═CH—), (—C::C—)]. Representative straight chain and branched alkenyls are those having about 2 to about 6 carbon atoms in the chain, for instance, ethylenyl, propylenyl, I-butenyl, 2-butenyl, isobutylenyl, I-pentenyl, 2-pentenyl, 3-methyl-1-butenyl, 2-methyl-2-butenyl, 2,3-dimethyl-2-butenyl, and the like.

The term “haloalkenyl” refers to a branched or straight-chain alkenyl as described above, substituted with one or more halogens.

The term “aryl” means an aromatic monocyclic or multi-cyclic (polycyclic) ring system of 6 to about 19 carbon atoms, for instance, about 6 to about 10 carbon atoms, and includes arylalkyl groups. Representative aryl groups include, but are not limited to, groups such as phenyl, naphthyl, azulenyl, phenanthrenyl, anthracenyl, fluorenyl, pyrenyl, triphenylenyl, chrysenyl, and naphthacenyl.

The term “arylalkyl” means an alkyl residue attached to an aryl ring. Examples are benzyl, phenethyl, and the like.

The term “heteroaryl” means an aromatic monocyclic or multi-cyclic ring system of about 5 to about 19 ring atoms, for instance, about 5 to about 10 ring atoms, in which one or more of the atoms in the ring system is/are element(s) other than carbon, for example, nitrogen, oxygen, and/or sulfur. As is well known to those skilled in the art, heteroaryl rings have less aromatic character than their all-carbon counter parts. Thus, for the purposes of the invention, a “heteroaryl” group need only have some degree of aromatic character. For instance, in the case of multi-cyclic ring systems, only one of the rings needs to be aromatic for the ring system to be defined as “heteroaryl”. Exemplary heteroaryls contain about 5 to 6 ring atoms. The prefix aza, oxa, thia, or thio before heteroaryl means that at least a nitrogen, oxygen, or sulfur atom, respectively, is present as a ring atom. A nitrogen, carbon, or sulfur atom in the heteroaryl ring may be optionally oxidized; the nitrogen may optionally be quaternized. Representative heteroaryls include, but are not limited to, purinyl, pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, quinolinyl, isoquinolinyl, quinazolinyl, cinnolinyl, phthalazinyl, quinoxalinyl, and the like.

The terms “cycloalkyl” and “cycloalkenyl” refer to a non-aromatic, saturated (cycloalkyl) or unsaturated (cycloalkenyl), mono- or multi-cyclic ring system of about 3 to about 8 carbon atoms, for instance, about 5 to about 7 carbon atoms. Exemplary cycloalkyl and cycloalkenyl groups include, without limitation, cyclopropyl, cyclobutyl, cyclopentyl, cyclohexyl, cycloheptyl, norbomyl, cyclopropenyl, cyclobutenyl, cyclopentenyl, cyclohexenyl, cyclophenyl, anti-bicyclopropane, syn-tricyclopropane, and the like.

As used herein, “heterocycle” or “heterocyclyl” refers to a stable 3- to 18 membered ring (radical) which is saturated, unsaturated, or aromatic, and which consists of carbon atoms and from one to five heteroatoms selected from the group consisting of nitrogen, oxygen and sulfur. For purposes of this invention, the heterocycle may be a monocyclic, bicyclic, or a polycyclic ring system, which may include fused, bridged, or spiro ring systems, including bicyclic rings in which any of the above heterocycles are fused to a benzene ring. The nitrogen, carbon, or sulfur atoms in the heterocycle may be optionally oxidized; the nitrogen atom may be optionally quaternized; and the ring may be partially or fully saturated. The heterocycle may be attached via any heteroatom or carbon atom. Heterocycles include heteroaryls as defined below. Examples of such heterocycles include, without limitation, morpholinyl, pyrrolidinonyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, tetrahydrothiopyranyl, and the like. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “acyl” refers to groups of from 1 to 8 carbon atoms of a straight, branched, or cyclic configuration, saturated, unsaturated, or aromatic, and combinations thereof, attached to the parent structure through a carbonyl functionality. One or more carbons in the acyl residue may be replaced by nitrogen, oxygen, or sulfur as long as the point of attachment to the parent remains at the carbonyl. Examples include acetyl (Ac), benzoyl, propionyl, isobutyryl, t-butoxycarbonyl, benzyloxycarbonyl, and the like.

The term “amino acid” refers to the fragment of an amino acid that remains following amide bond formation via reaction of the amino acid carboxyl group with an amino group of another molecule. The amino acid can be in D- or L-configuration. Suitable amino acids include α-amino acids, β-amino acids, γ-amino acids, 5-amino acids, and ε-amino acids, and include not only natural amino acids (i.e., those found in biological systems, including the twenty amino acids found in natural proteins), but also naturally-occurring variants of such amino acids, as well as synthetic amino acids and their analogues known to those skilled in the art. Exemplary amino acids include the twenty natural amino acids, 4-hydroxyproline, hydroxyysine, demosine, isodemosine, 3-methylhistidine, norvalin, beta-alanine, gamma-aminobutyric acid, citrulline, homocysteine, homoserine, ornithine, and methionine sulfone.

The term “pyrimidine” refers to a heteroaromatic compound containing a benzene ring with two carbon atoms replaced by two nitrogen atoms (diazine). For instance, the following moiety having the carbon atoms at positions 1 and 3 replaced by nitrogen atoms is considered a pyrimidine

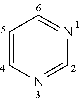

This term, as it is defined herein, also includes its isomeric forms of diazine, such as pyridazine, with the nitrogen atoms in positions 1 and 2; and pyrazine, with the nitrogen atoms in positions 1 and 4. The term “pyrimidine” also generally includes its analogues and derivatives. For instance, the natural nucleobases, cytosine (C), thymine (T), and uracil (D), are pyrimidine derivatives. The term “purine” refers to a heteroaromatic compound containing a pyrimidine ring fused to an imidazole ring. The term “purine” also generally includes its analogues and derivatives. For instance, the natural nucleobases, adenine (A) and guanine (G). Other examples of naturally occurring purine derivatives are hypoxanthine, xanthine, theobromine, caffeine, uric acid, and isoguanine. Exemplary purines and pyrimidines include those disclosed in U.S. Pat. No. 3,687,808; Concise Encyclopedia Of Polymer Science And Engineering, pages 858-859; 30 Kroschwitz, 1. 1, ed. John Wiley & Sons, 1990; and Englisch et al., Angewandte Chemie, International Edition, 1991, 30, 613, each of which is hereby incorporated by reference in its entirety.

The term “fatty acid” generally refers to a carboxylic acid with an aliphatic tail (chain). The aliphatic chain can be between about 2 and about 36 carbon atoms in length. Fatty acids can be saturated, unsaturated, or polyunsaturated. The aliphatic chain can be a linear or a branched chain. The term “fatty acid” may be used herein to refer to a “fatty acid derivative” which can include one or more different fatty acid derivatives, or mixtures of fatty acids derivatives. Exemplary fatty acids include unsaturated fatty acids, saturated fatty acids, and diacids; mono-, di-, and tri-glycerides of ascarosides that have a carboxylic acid functionality; hydroxy acids, co hydroxy acids, co-I hydroxy acids, di-hydroxy fatty acids (e.g., dihydroxy fatty acids that are omega- or omega-1 hydroxylated, as well as alpha- or beta-hydroxylated fatty acids).

The term “sugar” refers to a compound which is either a carbohydrate per se made up of one or more monosaccharide units having at least 5 carbon atoms (which may be linear, branched, or cyclic) with an oxygen, nitrogen, or sulfur atom bonded to each carbon atom; or a compound having as a part thereof a carbohydrate moiety made up of one or more monosaccharide units each having at least 5 carbon atoms (which may be linear, branched or cyclic), with an oxygen, nitrogen or sulfur atom bonded to each carbon atom. Representative sugars include the mono-, di-, tri-, and oligosaccharides containing from about 4-9 monosaccharide units, and polysaccharides such as starches, glycogen, cellulose, and polysaccharide gums. Exemplary monosaccharides include C, and above (e.g., C₅-C₈ or C₅-C₆) sugars; di- and trisaccharides include sugars having two or three monosaccharide units.

The term “monosaccharide” means a sugar molecule having a chain of 3-10 carbon atoms in the form of an aldehyde (aldose) or ketone (ketose). Suitable monosaccharides include both naturally occurring and synthetic monosaccharides. Suitable monosaccharides include trioses, such as glycerose and dihydroxyacetone; textroses such as erythrose and erythrulose; pentoses, such as xylose, arabinose, ribose, xylulose ribulose; methyl pentoses (6-deoxyhexoses), such as rhamnose and fucose; hexoses, such as ascarylose, glucose, mannose, galactose, fructose, and sorbose; and heptoses, such as glucoheptose, galamannoheptose, sedoheptulose, and mannoheptulose. Exemplary monosaccharides embrace radicals of allose, altrose, arabinose, cladinose, erythrose, erythrulose, fructose, Dfucitol, L-fucitol, fucosamine, fucose, fuculose, galactosamine, D-galactosaminitol, N-acetyl-galactosamine, galactose, glucosamine, N-acetyl-glucosamine, glucosaminitol, glucose, glucose-6-phosphate, gulose glyceraldehyde, L-glycero-D-mannos-heptose, glycerol, glycerone, gulose, idose, lyxose, mannosamine, mannose, mannose-6-phosphate, psicose, quinovose, quinovasamine, rhamnitol, rhamnosamine, rhamnose, ribose, ribulose, sedoheptulose, sorbose, tagatose, talose, tartaric acid, threose, xylose, and xylulose. The monosaccharide can be in D- or L-configuration. A typical monosaccharide used herein is hexose.

The monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or CvO replaced by C═S, or a ring oxygen of cyclic form replaced by sulfur), a seleno sugar, a telluro sugar, an aza sugar (ring carbon replaced by nitrogen), an imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth. Amino sugars include amino monosaccharides, such as galactosamine, glucosamine, mannosamine, fucosamine, quinovasamine, neuraminic acid, muramic acid, lactosediamine, acosamine, bacillosamine, daunosamine, desosamine, forosamine, garosamine, kanosamine, kansosamine, mycaminose, mycosamine, perosamine, pneumosamine, purpurosamine, rhodosamine. It is understood that the monosaccharide and the like can be further substituted.

The terms “disaccharide”, “trisaccharide”, and “polysaccharide” embrace radicals of abequose, acrabose, amicetose, amylopectin, amylose, apiose, arcanose, ascarylose, ascorbic acid, boivinose, cellobiose, cellotriose, cellulose, chacotriose, chalcose, chitin, colitose, cyclodextrin, cymarose, dextrin, 2-deoxyribose, 2-deoxyglucose, diginose, digitalose, digitoxose, evalose, evemitrose, fructoologosachharide, galto-oligosaccharide, gentianose, gentiobiose, glucan, glucogen, glycogen, hamamelose, heparin, inulin, isolevoglucosenone, isomaltose, isomaltotriose, isopanose, kojibiose, lactose, lactosamine, lactosediamine, laminarabiose, levoglucosan, levoglucosenone, ˜-maltose, maltriose, mannan-oligosaccharide, manninotriose, melezitose, melibiose, muramic acid, mycarose, 20 mycinose, neuraminic acid, nigerose, nojirimycin, moviose, oleandrose, panose, paratose, planteose, primeverose, raffinose, rhodinose, rutinose, sarmentose, sedoheptulose, solatriose, sophorose, stachyose, streptose, sucrose, a, a-trehalose, trehalosamine, turanose, tyvelose, xylobiose, umbelliferose, and the like. Further, it is understood that the “disaccharide”, “trisaccharide”, and “polysaccharide” and the like can be further substituted. Disaccharide also includes amino sugars and their derivatives, particularly, a mycaminose derivatized at the C-4′ position or a 4 deoxy-3-amino-glucose derivatized at the C-6′ position.

The term “polycyclic” or “multi-cyclic” used herein indicates a molecular structure having two or more rings, including, but not limited to, fused, bridged, or spiro rings.

The above “alkyl”, “alkenyl”, “cycloalkyl”, and “cycloalkenyl” radicals, as well as the ring system of the above aryl, heterocyclyl, or heteroaryl groups, may be optionally substituted.

The term “substituted” or “optionally substituted” is used to indicate that a group may have a substituent at each substitutable atom of the group (including more than one substituent on a single atom), provided that the designated atom's normal valency is not exceeded and the identity of each substituent is independent of the others. In accordance with the present invention, up to three H atoms in each residue can be replaced with alkyl, halogen, haloalkyl, alkyenyl, haloalkenyl, cycloalkyl, cycloalkenyl, hydroxy, alkoxy, acyl, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, aryl, heteroaryl, heterocyclyl, aryloxy, heteroaryloxy, a purine or pyridimine or an analogue or derivate thereof (as defined in “nucleobase”), or a sugar such as a monosaccharide having 5 or 6 carbon atoms (as defined in “monosaccharide”). “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious agent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstitutedsilyl, N3, SH, SCH3, C(O)CH3, CO2CH3, CO2H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH2CH3), fully substituted (e.g., —CF2CF3), monosubstituted (e.g., —CH2CH2F) or substituted at a level anywhere in-between fully substituted and monosubstituted (e.g., —CH2CF3). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

In the characterization of some of the substituents, certain substituents may combine to form rings. Unless stated otherwise, it is intended that such rings may exhibit various degrees of unsaturation (from fully saturated to fully unsaturated), may include heteroatoms, and may be substituted with other substituent groups as described above. The compounds described herein may contain one or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration; thus a carbon-carbon double bond depicted arbitrarily herein as trans may be Z, E, or a mixture of the two in any proportion.

The term “compounds” (e.g., “compounds including ascaroside) and equivalent expressions, are meant to embrace the prodrugs, the pharmaceutically acceptable salts, the oxides, the solvates, e.g. hydrates, and inclusion complexes of that compound, where the context so permits, as well as any stereoisomeric form, or a mixture of any such forms of that compound in any ratio, unless otherwise specified. Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed within the claims. Thus, in accordance with some embodiments of the invention, a compound as described herein, including in the contexts of biologically compatible compositions, methods of treatment, and compounds per se, is provided as the salt form. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The “quaternization” of any basic nitrogen-containing groups of the compounds disclosed herein is also contemplated. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides 25 including benzyl and phenethyl bromides. Water or oil-soluble or dispersible products may be obtained by such quaternization.

An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkoxy” as used herein, alone or in combination, refers to an alkyl ether radical, optionally substituted wherein the term alkyl is as defined below. Examples of alkyl ether radicals include methoxy, ethoxy, n-propoxy, isopropoxy, n-butoxy, iso-butoxy, sec-butoxy, tert-butoxy, and the like.

The term “alkylamino” as used herein, alone or in combination, refers to an alkyl group optionally substituted attached to the parent molecular moiety through an amino group. Alkylamino groups may be mono- or dialkylated, forming groups such as, for example, N-methylamino, N-ethylamino, N,N-dimethylamino, N,N-ethylmethylamino and the like.

The term “alkylthio” as used herein, alone or in combination, refers to an alkyl thioether (R—S—) radical wherein the term alkyl is as defined above and wherein the sulfur may be singly or doubly oxidized. Examples of alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynyl” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The term “amido” as used herein, alone or in combination, refer to an amino group as described below attached to the parent molecular moiety through a carbonyl group, or vice versa.

The term “amino” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryloxy” as used herein, alone or in combination, refers to an aryl group attached to the parent molecular moiety through an oxygen atom.

The term “polyether radical” means a polyether radical containing from 2 to 6 carbon atoms interrupted with at least one oxygen atom, such as methoxymethyl, ethoxymethyl or methoxyethoxymethyl radicals or methoxyethyl.

The terms “benzo” and “benz” as used herein, alone or in combination, refer to the divalent radical C6H4=derived from benzene. Examples include benzothiophene and benzimidazole.

The terms “carbamate” and “carbamoyl” as used herein, alone or in combination, refers to an ester of carbamic acid (—NHCOO—) which may be attached to the parent molecular moiety from either the nitrogen or acid end, and which may be optionally substituted as defined herein.

The term “carbonyl” as used herein, when alone includes formyl [—C(O)H] and in combination is a —C(O)— group.

The term “carboxy” as used herein, refers to —C(O)OH or the corresponding “carboxylate” anion, such as is in a carboxylic acid salt. An “O carboxy” group refers to a RC(O)O— group, where R is as defined herein. A “C carboxy” group refers to a —C(O)OR groups where R is as defined herein.

The term “chemical stability” according to the invention means that the content exhibits very little variation with respect to the initial content, namely, that the variation in content of active principle at the time T should not be less than 90% to more particularly than 95% of the initial content at TO.

The term “cyano” as used herein, alone or in combination, refers to —CN.

The term “cycloalkyl” or, alternatively, “carbocycle”, as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo-fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester” as used herein, alone or in combination, refers to a carboxy group bridging two moieties linked at carbon atoms.

The term “ether” as used herein, alone or in combination, refers to an oxygen atom bridging two moieties linked at carbon atoms.

The terms “halo” or “halogen” as used herein, alone or in combination, refers to fluorine, chlorine, bromine, or iodine. The term “halo alkyl” refers to a branched or straight-chain alkyl as described above, substituted with one or more halogens.

The term “haloalkyl” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CHF—), difluoromethylene (—CF2-), chloromethylene (—CHCl—) and the like.

The term “heteroalkyl” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH2-NH—OCH3.

The term “hydroxyl” as used herein, alone or in combination, refers to —OH.

The phrase “in the main chain” refers to the longest contiguous or adjacent chain of carbon atoms starting at the point of attachment of a group to the compounds of this invention.

The phrase “linear chain of atoms” refers to the longest straight chain of atoms independently selected from carbon, nitrogen, oxygen and sulfur.

The term “lower” as used herein, alone or in combination, means containing from 1 to and including 6 carbon atoms.

The term “negatively-charged ion” as used herein, refers to any negatively-charged ion or molecule, either inorganic (e.g., Cl—, Br—, I—) or organic (e.g., TsO— (i.e., tosylate)).

The term “nitro” as used herein, alone or in combination, refers to —NO2.

The term “perhaloalkyl” as used herein, alone or in combination, refers to an alkyl group where all of the hydrogen atoms are replaced by halogen atoms.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amino group, and the term alkoxyalkyl would represent an alkoxy group attached to the parent molecule through an alkyl group.

When a group is defined to be “null,” what is meant is that said group is absent.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “imaging agent” as used herein refers to any moiety useful for the detection, tracing, or visualization of a compound of the invention when coupled thereto. Imaging agents include, e.g., an enzyme, a fluorescent label (e.g., fluorescein), a luminescent label, a bioluminescent label, a magnetic label, a metallic particle (e.g., a gold particle), a nanoparticle, an antibody or fragment thereof (e.g., a Fab, Fab′, or F(ab′)2 molecule), and biotin. An imaging agent can be coupled to a compound of the invention by, for example, a covalent bond, ionic bond, van der Waals interaction or a hydrophobic bond. An imaging agent of the invention can be a radiolabel coupled to a compound of the invention, or a radioisotope incorporated into the chemical structure of a compound of the invention. Methods of detecting such imaging agents include, but are not limited to, positron emission tomography (PET), X-ray computed tomography (CT) and magnetic resonance imaging (MRI).

The phrase “therapeutically effective” is intended to qualify the amount of active ingredients used in the treatment of a disease or disorder. This amount will achieve the goal of reducing or eliminating the disease or disorder.

The term “therapeutically acceptable” refers to those compounds (or salts, esters, prodrugs, tautomers, zwitterionic forms, etc. thereof) which are suitable for use in contact with the tissues of patients without undue toxicity, irritation, and allergic response, are commensurate with a reasonable benefit/risk ratio, and are effective for their intended use.

As used herein the term “microbiota” (equivalently referred to herein as “microbiome”) refers to the assemblage of microorganisms (bacteria, archaea, eukaryotes, and viruses) present in a defined environment. The microbiota may vary according to its surrounding environment. The term microbiota is typically preceded by the name of the environment in which it is located. For example, “gut microbiota” refers to the microbiota in the intestinal tract.

As used herein, reference to “treatment” of a patient is intended to include prophylaxis. The term “patient” means mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “patient” does not denote a particular age or sex.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology, Testa, Bernard and Wiley-VHCA, Zurich, Switzerland 2003. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bio-available by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug is a compound that is administered as an ester (the “prodrug”), but then is metabolically hydrolyzed to the carboxylic acid, the active entity. Additional examples include peptidyl derivatives of a compound.

The term “compounds of the invention,” and equivalent expressions, are meant to embrace the compounds, as well as any stereoisomeric form, or a mixture of any such forms of that compound in any ratio, unless otherwise specified. Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed within the claims. For the sake of clarity, particular instances when the context so permits are sometimes indicated in the text, but these instances are purely illustrative and it is not intended to exclude other instances when the context so permits.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixtures and separate individual isomers.

Some of the compounds may also exist in tautomeric forms. Such forms, although not explicitly indicated in the formulae described herein, are intended to be included within the scope of the present disclosure.

The compounds of the invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Stahl, P. Heinrich, Pharmaceutical Salts: Properties, Selection, and Use, Wiley-VCHA, Zurich, Switzerland (2002).

The term “therapeutically acceptable salt” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethanesulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid. The novel compounds described herein can be prepared in a form of pharmaceutically acceptable salts that will be prepared from nontoxic inorganic or organic bases including but not limited to aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally-occurring substituted amines, cyclic amines, and basic ion exchange resins, such as arginine, betaine, caffeine, choline, ethylamine, 2-diethylaminoethano, 1,2-dimethylaminoethanol, ethanolamine, ethylenediamine, N-ethyl-morpholine, N-ethylpiperidine, glucamine, glucosamine, Histidine, hydroxylamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, and tromethamine.

If the compounds of the invention are basic, salts could be prepared in a form of pharmaceutically acceptable salts that will be prepared from nontoxic inorganic or organic acids including but not limited to hydrochloric, hydrobromic, phosphoric, sulfuric, tartaric, citric, acetic, fumaric, alkylsulphonic, naphthalenesulphonic, para-toluenesulphonic, camphoric acids, benzenesulfonic, benzoic, camphorsulfonic, citric, ethanesulfonic, gluconic, glutamic, isethonic, lactic, maleic, malic, mandelic, methanesulfonic, mucic, nitric, pamoic, pantothenic, phosphoric, and succinic.

While it may be possible for the compounds of the invention to be administered as the raw chemical, it is also possible to present them as a pharmaceutical formulation. Accordingly, the present invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

Formulations that may be suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the present invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds described herein may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the compounds described herein, the compounds of the invention may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compounds described herein may take the form of tablets, lozenges, pastilles, or gels formulated in conventional manner. Such compositions may comprise the active ingredient in a flavored basis such as sucrose and acacia or tragacanth.

The compounds described herein may also be formulated in rectal compositions such as suppositories or retention enemas, e.g., containing conventional suppository bases such as cocoa butter, polyethylene glycol, or other glycerides.

The compounds described herein may be administered topically, that is by non-systemic administration. This includes the application of a compound externally to the epidermis or the buccal cavity and the instillation of such a compound into the ear, eye and nose, such that the compound does not significantly enter the bloodstream. In contrast, systemic administration refers to oral, intravenous, intraperitoneal and intramuscular administration.

Formulations suitable for topical administration include solid, liquid or semi-liquid preparations suitable for penetration through the skin to the site of infection such as gels, liniments, lotions, creams, ointments or pastes, and drops suitable for administration to the eye, ear or nose. In some examples the active ingredient may comprise, for topical administration, from 0.001% to 40% w/w, for instance from 1% to 5% by weight of the formulation (e.g., 0.001% to 35%, 0.002% to 30%, 0.01% to 25%, 0.05% to 20%, 0.1% to 15%, 0.1% to 12.5%, 0.5% to 10%, 0.5% to 8%, 1% to 7%, 1% to 6%, 1% to 5%, etc.). It may however comprise more than 10% w/w (e.g., 20% or less, 25% or less, 30% or less, 35% or less, 40% or less, 45% or less, 50% or less, etc. and/or in some variations greater than 0.001%, greater than 0.01%, greater than 0.1%, greater than 1%, etc.).

Via the topical route, the compounds described herein may be in the form of liquid or semi liquid such as ointments, or in the form of solid such as powders. It may also be in the form of suspensions such as polymeric microspheres, or polymer patches and hydrogels allowing a controlled release. This topical composition may be in anhydrous form, in aqueous form or in the form of an emulsion. The compounds may be used topically at a concentration of between 0.001% and 10% by weight (e.g., between 0.01% and 1% by weight), relative to the total weight of the composition. In some variations, the compounds may be used topically at greater than 10% by weight (e.g., 20% or less, 25% or less, 30% or less, 35% or less, 40% or less, 45% or less, 50% or less, etc.).

For administration by inhalation, the compounds described herein may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations include those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example, those suitable for oral administration may include flavoring agents.

The compounds described herein may be administered orally. Tablets or other forms of presentation provided in discrete units may conveniently contain an amount of compound of the invention which is effective at such dosage or as a multiple of the same. For example, compounds described herein can be administered at a daily dose of about 0.001 mg/kg to 100 mg/kg of body weight, in 1 to 5 dosage intakes (e.g., 1/day, 2/day, 3/day, 4/day, 5/day, etc.). In some variations, compounds can be used systemically, at a concentration generally of between 0.001% and 10% by weight and preferably between 0.01% and 1% by weight, relative to the weight of the composition.

The one or more ascarosides may be collectively or separately considered the active ingredient (or if separately, active ingredients) that may be combined with the carrier materials to produce a single dosage form may vary depending upon the host treated and the particular mode of administration.

The compounds described herein can be administered in various modes, e.g. orally, topically, etc. The precise amount of compound administered to a patient may be the responsibility of the attendant physician. The specific dose level for any particular patient may depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein, or a pharmaceutically acceptable salt, ester, or prodrug thereof, in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for pain involving administration of one of the compounds described herein, increased therapeutic benefit may result by also providing the patient with another therapeutic agent for pain. In any case, regardless of the disease, disorder or condition being treated, the overall benefit experienced by the patient may simply be additive of the two therapeutic agents or the patient may experience a synergistic benefit.

Specific, non-limiting examples of possible combinations with the compounds described herein may include use of the compounds together with inert or active compounds, or other drugs including wetting agents, flavor enhancers, preserving agents, stabilizers, humidity regulators, pH regulators, osmotic pressure modifiers, emulsifiers, UV-A and UV-B screening agents, antioxidants, depigmenting agents such as hydroquinone or kojic acid, emollients, moisturizers, for instance glycerol, PEG 400, or urea, antiseborrhoeic or antiacne agents, such as S-carboxymethylcysteine, S-benzylcysteamine, salts thereof or derivatives thereof, or benzoyl peroxide, antibiotics, for instance erythromycin and tetracyclines, chemotherapeutic agent, for example, paclitaxel, antifungal agents such as ketoconazole, agents for promoting regrowth of the hair, for example, minoxidil (2,4-diamino-6-piperidinopyrimidine 3-oxide), non-steroidal anti-inflammatory agents, carotenoids, and especially p-carotene, antipsoriatic agents such as anthralin and its derivatives, eicosa-5,8,11,14-tetraynoic acid and eicosa-5,8,11-triynoic acid, and esters and amides thereof, retinoids, e.g., RAR or RXR receptor ligands, which may be natural or synthetic, corticosteroids or oestrogens, alpha-hydroxy acids and a-keto acids or derivatives thereof, such as lactic acid, malic acid, citric acid, and also the salts, amides or esters thereof, or p-hydroxy acids or derivatives thereof, such as salicylic acid and the salts, amides or esters thereof, ion-channel blockers such as potassium-channel blockers, or alternatively, more particularly for the pharmaceutical compositions, in combination with medicaments known to interfere with the immune system, anticonvulsant agents include, and are not limited to, topiramate, analogs of topiramate, carbamazepine, valproic acid, lamotrigine, gabapentin, phenytoin and the like and mixtures or pharmaceutically acceptable salts thereof. A person skilled in the art will take care to select the other compound(s) to be added to these compositions such that the advantageous properties intrinsically associated with the compounds of the invention are not, or are not substantially, adversely affected by the envisaged addition.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, methods for treating diseases, disorders, conditions, or symptoms in a patient (e.g., a human or animal) in need of such treatment are presented herein, the methods comprising the step of administering to the patient an amount of a compound of the invention effective to reduce or prevent the disease, disorder, condition, or symptom, in combination with at least one additional agent for the treatment of said disorder that is known in the art.

The term “ascaroside” may refer to a compound of Formula I:

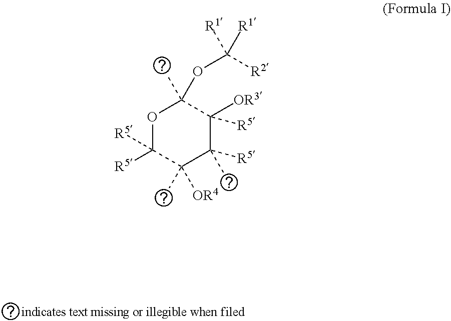

or a pharmaceutical equivalent, derivative, analog, and/or salt thereof. As readily apparent to one of skill in the art, the compound may be further defined by various R groups, where: R^{1′} is H, —C(R′)₃, —OR, —N(R′)₂, halogen, an alkyl, a haloalkyl, an alkenyl, or a haloalkenyl; where each R is independently H, halogen, an alkyl, or an alkenyl; R^{1′} is absent, H, —C(R′)₃, —OR, —N(R′)₂, halogen, an alkyl, a haloalkyl, an alkenyl, or a haloalkenyl; where each R′ is independently H, halogen, an alkyl, or an alkenyl; R^{2′} is a moiety of formula:

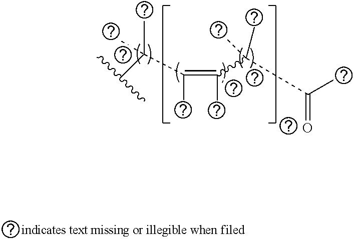

where:
each R^{1′} is independently H, —C(R′)₃, —OR, —N(R′)₂, halogen, an alkyl, a haloalkyl, an alkenyl, or a haloalkenyl; where each R is independently H, halogen, an alkyl, or an alkenyl;
R^{5′} is H, —OH, —OR^{6′}, —OCR^{6′}R^{7′}R^{8′}, —CR^{6′}R^{7′}R^{8′}, —NH₂, —NHR^{6′}, —NR^{6′}R^{7′}, halogen, an alkyl, a haloalkyl, an alkenyl, a haloalkenyl, an aryl, a heteroaryl, an arylalkyl, a heterocyclyl, a cycloalkyl, a cycloalkenyl, an acyl, an amino acid, a nucleoside, a monosaccharide having 5 or 6 carbon atoms, or a bond connecting to R^{3′} or R^{4′} of another unit of Formula III;
where:
R^{6′} and R^{7′} are each independently H, —CR′₃, —OR, —N(R′)₂, halogen, an alkyl, a haloalkyl, an alkenyl, a haloalkenyl, an aryl, a heteroaryl, a heterocyclyl, a cycloalkyl, or a cycloalkenyl, where the alkyl, alkenyl, aryl, heteroaryl, heterocyclyl, cycloalkyl, or cycloalkenyl is optionally substituted with one or more substituents independently selected from the group consisting of —OR^{8′}, —C(O)R^{8′}, —NHC(O)R^{8′}, an alkyl, a haloalkyl, an aryl, a heteroaryl, a heterocyclyl, and a cycloalkyl;
where:
each R′ is independently H, halogen, an alkyl, or an alkenyl; and
R^{8′} is H, —C(R′)₃, —[C(R′)₂]ₙ₄NHC(O)R^{9′}, [C(R′)₂]ₙ₄C(O)(NH)R^{9′}, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR^{9′}, —C(O)R^{9′}, —NHC(O)R^{9′}, halogen, an alkyl, a haloalkyl, an aryl, a heteroaryl, a heterocyclyl, a cycloalkyl, and a monosaccharide having 5 or 6 carbon atoms;
where:
each R′ is independently H, halogen, an alkyl, or an alkenyl;
R^{9′} is H, —C(R′)₃, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR′, —C(O)R′, halogen, an alkyl, a haloalkyl, an aryl, a heteroaryl, a heterocyclyl, and a cycloalkyl; where each R′ is independently H, halogen, an alkyl, or an alkenyl; and
n⁴ is an integer of 1 to 30; and
R^{8′} is H, —C(R′)₃, —[C(R′)₂]ₙ₄NHC(O)R^{9′}, —[C(R′)₂]ₙ₄C(O)(NH)R^{9′}, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of C(R′)₃, —OR^{9′}, —C(O)R^{9′}, —NHC(O)R^{9′}, halogen, an alkyl, a haloalkyl, an aryl, a heteroaryl, a heterocyclyl, a cycloalkyl, and a monosaccharide having 5 or 6 carbon atoms;
where:
each R′ is independently H, halogen, an alkyl, or an alkenyl;
R^{9′} is H, —C(R′)₃, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR′, —C(O)R′, halogen, an alkyl, a haloalkyl, an aryl, a heteroaryl, a heterocyclyl, and a cycloalkyl; where each R′ is independently H, halogen, an alkyl, or an alkenyl; and
n⁴ is an integer of 1 to 30;
n′, n², and n³ are each independently an integer of 0 to 30;
n⁴ is an integer of 1 to 30; and
the sum of n¹, each n², and each n³ is 1 to 30;
R^{3′} and R^{4′} are each independently H, —CR^{6′}R^{7′}R^{8′}, —C(O)R^{8′}, an alkyl, a haloalkyl, an alkenyl, a haloalkenyl, an aryl, a heteroaryl, a heterocyclyl, a cycloalkyl, a cycloalkenyl, an acyl, an amino acid, a nucleoside, a monosaccharide having 5 or 6 carbon atoms, or a bond connecting to R^{5′} of another unit of Formula I;
where:
R^{6′} and R^{7′} are each independently H, —CR′₃, —OR′, —N(R′)₂, halogen, an alkyl, a haloalkyl, an alkenyl, a haloalkenyl, an aryl, a heteroaryl, a heterocyclyl, a cycloalkyl, or a cycloalkenyl, where the alkyl, alkenyl, aryl, heteroaryl, heterocyclyl, cycloalkyl, or cycloalkenyl is optionally substituted with one or more substituents independently selected from the group consisting of OR^{8′}, —C(O)R^{8′}, —NHC(O)R^{8′}, an alkyl, a haloalkyl, an aryl, a heteroaryl, a heterocyclyl, and a cycloalkyl;
where:
each R′ is independently H, halogen, an alkyl, or an alkenyl; and
R^{8′} is H, —C(R′)₃, [C(R′)₂]ₙ₄NHC(O)R^{9′}, —[C(R′)₂]ₙ₄C(O)(NH)R^{9′}, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR^{9′}, —C(O)R^{9′}, —NHC(O)R^{9′}, halogen, an alkyl, a haloalkyl, an aryl, a heteroaryl, a heterocyclyl, a cycloalkyl, and a monosaccharide having 5 or 6 carbon atoms;
where:
each R′ is independently H, halogen, an alkyl, or an alkenyl;
R^{9′} is H, —C(R′)₃, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of C(R′)₃, —OR′, —C(O)R′, halogen, an alkyl, a haloalkyl, an aryl, a heteroaryl, a heterocyclyl, and a cycloalkyl; where each R′ is independently H, halogen, an alkyl, or an alkenyl; and
n⁴ is an integer of 1 to 30; and
R^{8′} is H, —C(R′)₃, —[C(R′)₂]ₙ₄NHC(O)R^{9′}, —[C(R′)₂]ₙ₄C(O)(NH)R^{9′}, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR^{9′}, —C(O)R^{9′}, —NHC(O)R^{9′}, halogen, an alkyl, a haloalkyl, an aryl, a heteroaryl, a heterocyclyl, a cycloalkyl, and a monosaccharide having 5 or 6 carbon atoms;
where:
each R′ is independently H, halogen, an alkyl, or an alkenyl;
R^{9′} is H, —C(R′)₃, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR′, —C(O)R′, halogen, an alkyl, a haloalkyl, an aryl, a heteroaryl, a heterocyclyl, and a cycloalkyl; where each R′ is independently H, halogen, an alkyl, or an alkenyl; and
n⁴ is an integer of 1 to 30; and
each R^{5′} is independently H, —OH, —OR^{6′}, —OCR^{6′}R^{7′}R^{8′}, —CR^{6′}R^{7′}R^{8′}, —NH₂, —NHR^{6′}, —NR^{6′}R^{7′}, halogen, an alkyl, a haloalkyl, an alkenyl, a haloalkenyl, an aryl, a heteroaryl, an arylalkyl, a heterocyclyl, a cycloalkyl, a cycloalkenyl, an acyl, an amino acid, a nucleoside, a monosaccharide having 5 or 6 carbon atoms, or a bond connecting to R^{3′} or R^{4′} of another unit of Formula I;
where:
R^{6′} and RT are each independently H, —CR′₃, —OR′, —N(R′)₂, halogen, an alkyl, a haloalkyl, an alkenyl, a haloalkenyl, an aryl, a heteroaryl, a heterocyclyl, a cycloalkyl, or a cycloalkenyl, where the alkyl, alkenyl, aryl, heteroaryl, heterocyclyl, cycloalkyl, or cycloalkenyl is optionally substituted with one or more substituents independently selected from the group consisting of OR^{8′}, —C(O)R^{8′}, —NHC(O)R^{8′}, an alkyl, a haloalkyl, an aryl, a heteroaryl, a heterocyclyl, and a cycloalkyl;
where:
each R′ is independently H, halogen, an alkyl, or an alkenyl; and
R^{8′} is H, —C(R′)₃, —[C(R′)₂]ₙ₄NHC(O)R^{9′}, —[C(R′)₂]ₙ₄C(O)(NH)R^{9′}, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR^{9′}, —C(O)R^{9′}, —NHC(O)R^{9′}, halogen, an alkyl, a haloalkyl, an aryl, a heteroaryl, a heterocyclyl, a cycloalkyl, and a monosaccharide having 5 or 6 carbon atoms;
where:
each R′ is independently H, halogen, an alkyl, or an alkenyl;
R^{9′} is H, —C(R′)₃, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR′, —C(O)R′, halogen, an alkyl, a haloalkyl, an aryl, a heteroaryl, a heterocyclyl, and a cycloalkyl; where each R′ is independently H, halogen, an alkyl, or an alkenyl; and n⁴ is an integer of 1 to 30; and
R^{8′} is H, —C(R′)₃, —[C(R′)₂]ₙ₄NHC(O)R^{9′}, —[C(R′)₂]ₙ₄C(O)(NH)R^{9′}, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of C(R′)₃, —OR^{9′}, —C(O)R^{9′}, —NHC(O)R^{9′}, halogen, an alkyl, a haloalkyl, an aryl, a heteroaryl, a heterocyclyl, a cycloalkyl, and a monosaccharide having 5 or 6 carbon atoms;
where:
each R′ is independently H, halogen, an alkyl, or an alkenyl;
R^{9′} is H, —C(R′)₃, —OR′, —N(R′)₂, halogen, an alkyl, a haloalkyl, an alkenyl, a haloalkenyl, an aryl, a heteroaryl, a heterocyclyl, a cycloalkyl, a cycloalkenyl, a purine, a pyrimidine, or a monosaccharide having 5 or 6 carbon atoms, where the alkyl, alkenyl, aryl, heteroaryl, heterocyclyl, cycloalkyl, cycloalkenyl, purine, pyrimidine, or monosaccharide is optionally substituted with one or more substituents independently selected from the group consisting of —C(R′)₃, —OR′, —C(O)R′, halogen, an alkyl, a haloalkyl, an aryl, a heteroaryl, a heterocyclyl, and a cycloalkyl; where each R′ is independently H, halogen, an alkyl, or an alkenyl; and n⁴ is an integer of 1 to 30.

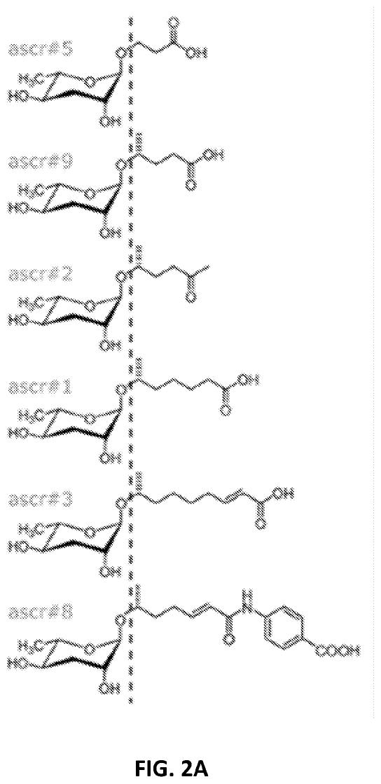

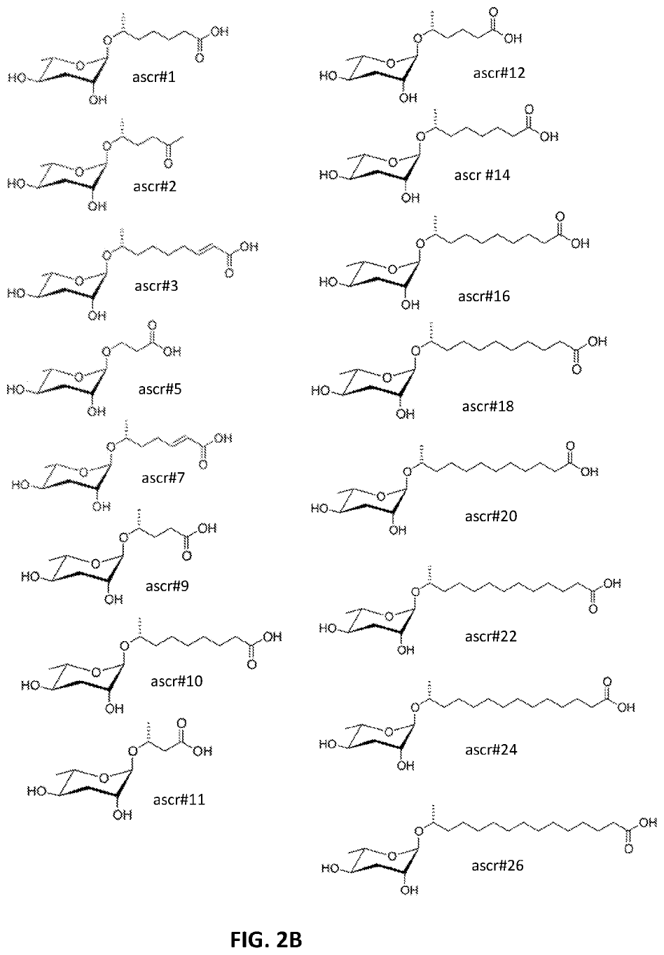

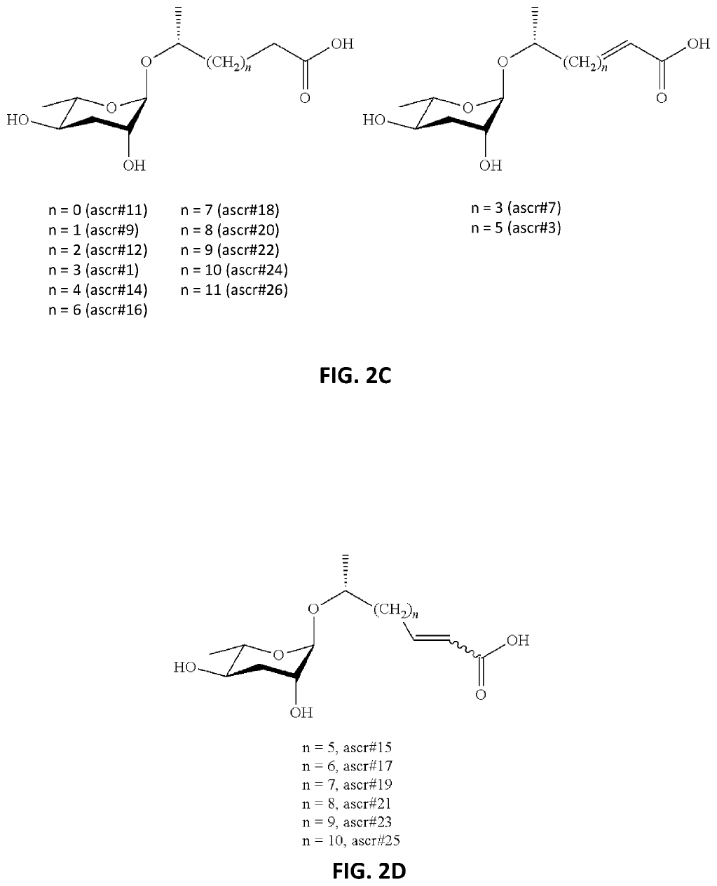

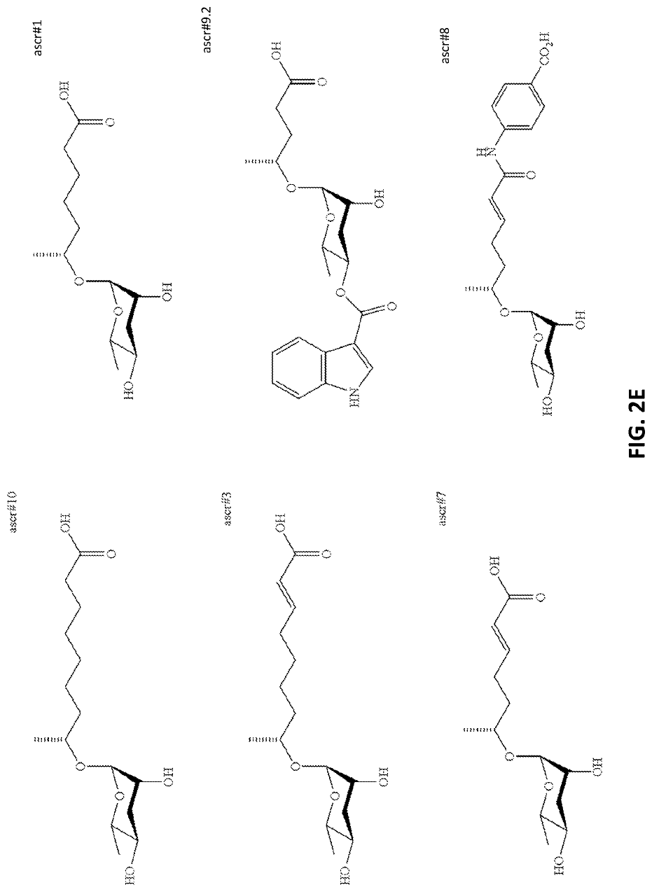

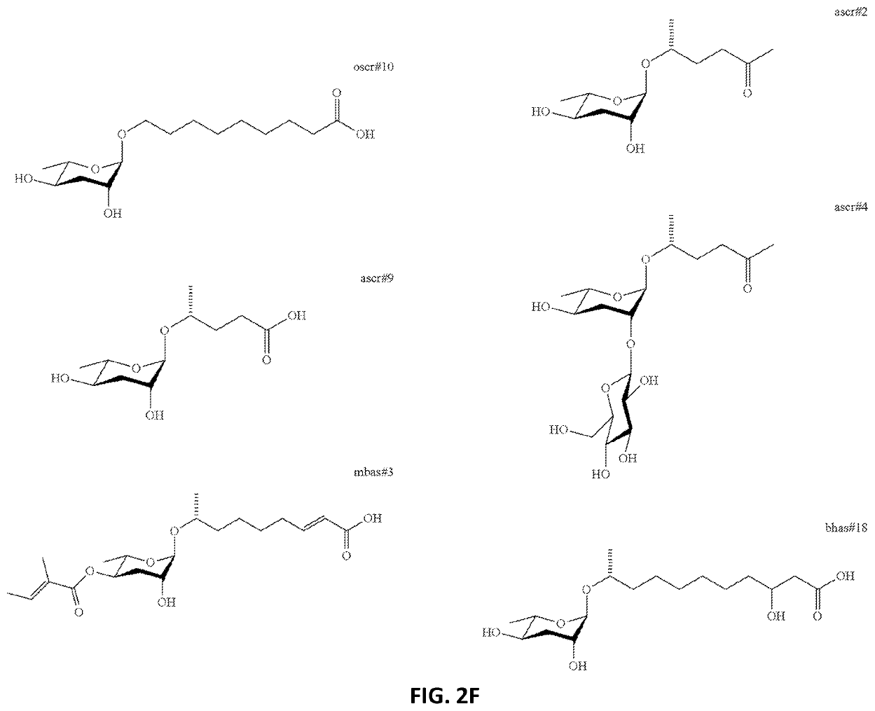

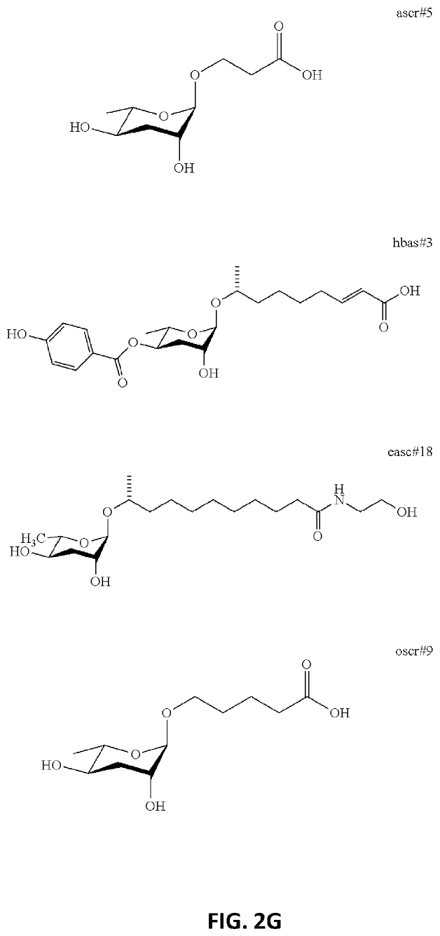

In some variations, the ascarosides for use in the methods and compositions described herein include those shown in FIGS. 2B-2E, as well as compounds that are structurally identical to the compounds provided below except for the number of carbon atoms in the fatty acid-like side chain (e.g., from between 3 and 24 carbons). Compounds with fatty acid-like side chains containing between 3 and 24 carbon atoms are contemplated for use in the invention.

In some variations, ascarosides for use in the methods and compositions described herein include those compounds of Formula II, which is a subset of Formula I:

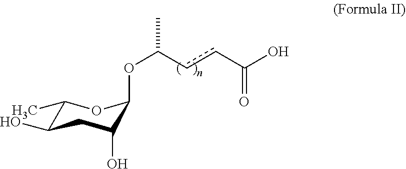

where [symbol] represents a double or single bond, ( )ₙ represents (CH2)ₙ where n may be 1-12. The naturally-occurring Ascarosides shown in FIG. 2B are a subset of the Formula II ascarosides.

Described herein are compositions including one or more ascaroside having the general form of Formula I. Also described herein are compositions including one or more ascaroside having the general form of Formula II (including those in FIGS. 2B and 2C). For example, the compositions described herein may include one or more of:

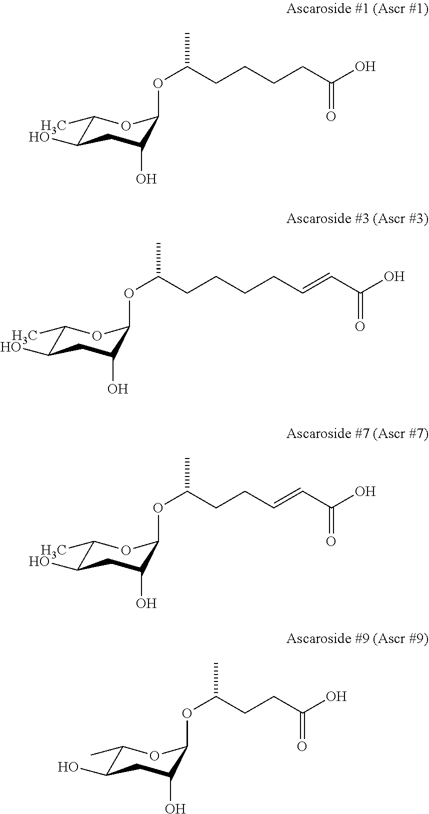

In some variations, ascarosides for use in the methods and compositions described herein include naturally-occurring Ascarosides (ascrs #1, #2, #3, #5, #7, #9, #10, #11, #12, #14, #16, #18, #20, #22, #24, and #26) that are secreted by free living or parasitic worms (see, e.g., FIG. 2B).

In some variations the ascarosides for use in the methods and compositions described herein include Ascaroside derivatives, having the formula of Formula III or Formula IV, described in greater detail below.

Described herein are methods of modifying a mammalian microbiome. Any of these methods may include providing a composition including one or more ascarosides to be exposed to the patient's gut microbiome for a dosage period. For example, described herein are methods of preventing, alleviating, or treating disorders related to microbiome imbalance, such as: dysbiosis, irritable bowel syndrome, irritable bowel syndrome with constipation, irritable bowel syndrome with diarrhea, functional constipation, functional diarrhea, chronic diarrhea, and/or chronic constipation. In some aspects, the method comprises administering to the subject a composition comprising a compound of Formula I or a pharmaceutically acceptable salt thereof or product thereof. The method comprises administering to the subject a composition comprising a compound of Formula II or a pharmaceutically acceptable salt thereof or product thereof. In some aspects, the method comprises administering to the subject a composition comprising a compound of Formula II with prebiotics, probiotics, or antibiotics. For example, any of the compositions described herein may include one or more of: Ascr #1, Ascr #3, Ascr #7 and/or Ascr #9, and sub-combinations thereof. The ascaroside(s) in the composition may be formed from an extract, or more preferably, synthesized.

In some aspects, the method comprises administering to the subject a composition comprising a naturally-occurring Ascaroside or a pharmaceutically acceptable salt thereof or product thereof. The method comprises administering to the subject a composition comprising a naturally-occurring Ascaroside (e.g., e.g., ascr #1, ascr #2, ascr #3, ascr #5, ascr #7, ascr #9, ascr #10, ascr #11, ascr #12, ascr #14, ascr #16, ascr #18, ascr #20, ascr #22, ascr #24, and ascr #26), or a pharmaceutically acceptable salt thereof or product thereof. In some aspects, the method comprises administering to the subject a composition comprising a naturally-occurring Ascaroside with prebiotics, probiotics, or antibiotics. For example, any of the compositions described herein may include one or more of: ascr #1, ascr #2, ascr #3, ascr #5, ascr #7, ascr #9, ascr #10, ascr #11, ascr #12, ascr #14, ascr #16, ascr #18, ascr #20, ascr #22, ascr #24, and ascr #26, and sub-combinations thereof. The ascaroside(s) in the composition may be formed from an extract, or more preferably, synthesized.

In some aspects, the method comprises administering to the subject a composition comprising an Ascaroside derivative or a pharmaceutically acceptable salt thereof or product thereof. The method comprises administering to the subject a composition comprising an Ascaroside derivative, or a pharmaceutically acceptable salt thereof or product thereof. In some aspects, the method comprises administering to the subject a composition comprising an Ascaroside derivative with prebiotics, probiotics, or antibiotics.

### Ascaroside Derivatives

The Ascaroside derivatives described herein may comprise a compound of Formula III or IV, described below, and may be enantioenriched at the side chain methyl group stereogenic center. The level of enantioenrichment of a compound may be expressed as enantiomeric excess (ee). The ee of a compound may be measured by dividing the difference in the fractions of the enantiomers by the sum of the fractions of the enantiomers. For example, if a compound is found to comprise 98% (S)-enantiomer, and 2% (R)-enantiomer, then the ee of the compound is (98−2)/(98+2), or 96%. In certain embodiments, the compound of Formula III has about 5% ee or greater, 10% ee or greater, 15% ee or greater, 20% ee or greater, 25% ee or greater, 30% ee or greater, 40% ee or greater, 50% ee or greater, 60% ee or greater, 70% ee or greater, about 80% ee, about 85% ee, about 88% ee, about 90% ee, about 91% ee, about 92% ee, about 93% ee, about 94% ee, about 95% ee, about 96% ee, about 97% ee, about 98% ee, about 99% ee, or above about 99% ee, even where this % ee is greater than the % ee of the starting material, such as 0% ee (racemic). In some embodiments, the compound of Formula III or IV is enantioenriched. In some embodiments, the compound of Formula III or IV is enantiopure e.g., has the structure of compound 1 or 10. In embodiments where the starting material has more than one stereocenter, reactions of the disclosure may enrich the stereocenter bearing a methyl group relative to the enrichment at this center, if any, of the starting material, and substantially independently of the stereochemical disposition/enrichment (de) of any other stereocenters of the molecule. For example, a compound of Formula III or IV described herein may have 5% de or greater, 10% de or greater, 15% de or greater, 20% de or greater, 25% de or greater, 30% de or greater, 40% de or greater, 50% de or greater, 60% de or greater, 70% de or greater, 80% de or greater, 90% de or greater, 95% de or greater, or even 98% de or greater at the stereocenter of the compound of Formula III bearing a methyl group.

For example, an Ascaroside derivative compound may have the structure of Formula III:

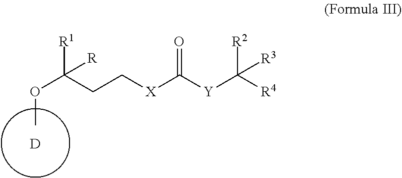

wherein Ring D is selected from:

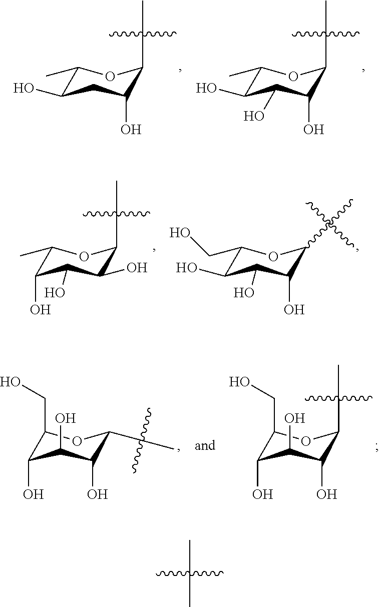

represents the point of attachment of Ring D to the oxygen atom;

X is —C(R⁵)₂—C(R⁵)₂— or E or Z —CH═CH—;

Y is O or NR⁶;

R is H or CH₃,

R¹ is H or CH₃;

or, R and R¹, taken together with the carbon atom to which they are bonded, form a 3- or 4-membered carbocyclic ring;

R² is H or CH₃;

R³ is H or CH₃;

R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆alkyl;

R⁵, independently for each occurrence, is H or OH,

or two instances of R⁵, taken together with the carbon atom or carbon atoms to which they are bonded, form a 3-membered carbocyclic ring; and

R⁶ is H or C₁-C₆ alkyl,

or, when Y is NR⁶, R⁴ and NR⁶, taken together with the carbon atom to which they are bonded, form a 5- or 6-membered heterocyclic ring.

In some embodiments, the compound of Formula III is selected from

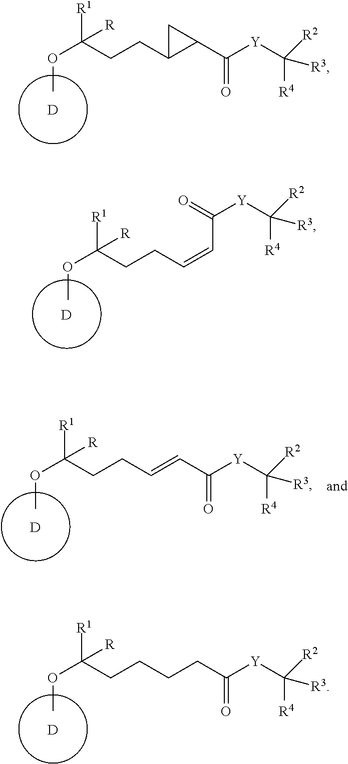

In some embodiments, X is —C(R⁵)₂—C(R⁵)₂—. In some embodiments, X is E or Z —CH═CH—. In some embodiments, X is —C(H)₂—C(H)₂—. In some embodiments, X is —C(H)(OH)—C(H)₂—.

In some embodiments, X —C(R⁵)₂—C(R⁵)₂, two instances of R⁵, taken together with the carbon atom or carbon atoms to which they are bonded, form a 3-membered carbocyclic ring.

In some embodiments, X is

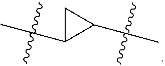 [SMILES: *C1CC1*]

In some embodiments, X is

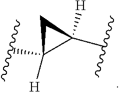 [SMILES: *[C@@H]1C[C@H]1*]

In some embodiments, X is

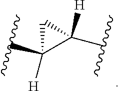 [SMILES: *[C@H]1C[C@@H]1*]

In some embodiments, X is

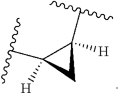 [SMILES: *[C@@H]1C[C@@H]1*]

In some embodiments, X is

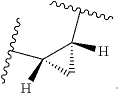 [SMILES: *[C@@H]1C[C@@H]1*]

In some embodiments, Y is NR⁶. In some embodiments, Y is NR⁶, wherein R⁶ is H. In some embodiments, Y is NR⁶, wherein R⁶ is C₁-C₆ alkyl. In some embodiments, Y is NR⁶, wherein R⁶ is CH₃. In some embodiments, Y is NR⁶, wherein R⁶ is CH₂CH₃. In some embodiments, Y is NR⁶, wherein R⁴ and NR⁶, taken together with the carbon atom to which they are bonded, form a 5- or 6-membered heterocyclic ring. In some embodiments, Y is O.

In some embodiments, R and R¹ are each CH₃. In some embodiments, R and R¹ are each CH₃. In some embodiments, R is CH₃; and R¹ is H. In some embodiments, R and R¹, taken together with the carbon atom to which they are bonded, form a cyclopropyl. In some embodiments, R and R¹, taken together with the carbon atom to which they are bonded, form a cyclobutyl.

In some embodiments, R² is H; R³ is H or CH₃; and R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl, or, when Y is NR⁶, R⁴ and NR⁶, taken together with the carbon atom to which they are bonded, form a 5- or 6-membered heterocyclic ring. In some embodiments, R², R³ and R⁴ are each CH₃. In some embodiments, R² is H; R³ is CH₃; and R⁴ is CH₃. In some embodiments, R² is H; R³ is H; and R⁴ is CH₂CH₃. In some embodiments, R² is H; R³ is H; and R⁴ is CH(CH₂CH₃)₂. In some embodiments, R² is H; R³ is H; and R⁴ is CH(CH₂CH₃)₂. In some embodiments, R² is H; R³ is H; and R⁴ is cyclohexyl. In some embodiments, R² is H; R³ is H; and R⁴ is phenyl. In some embodiments, R² is H; R³ is H; and R⁴ is pyridinyl.

In some embodiments, the compound of Formula III is selected from

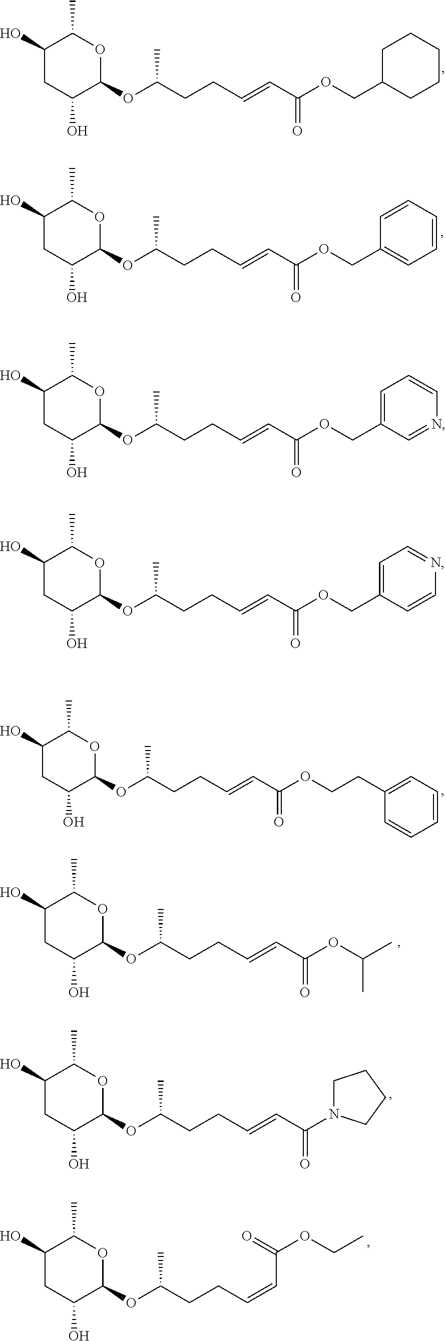

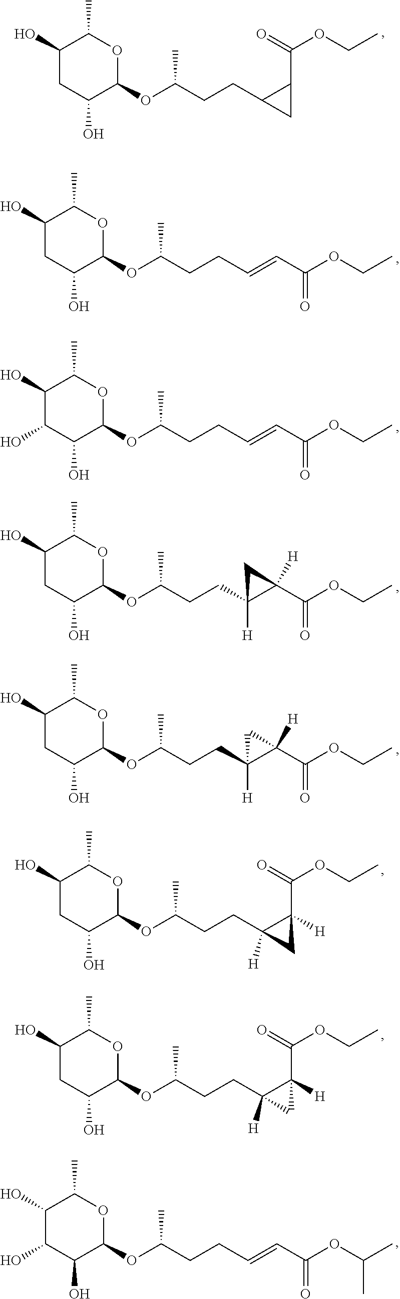

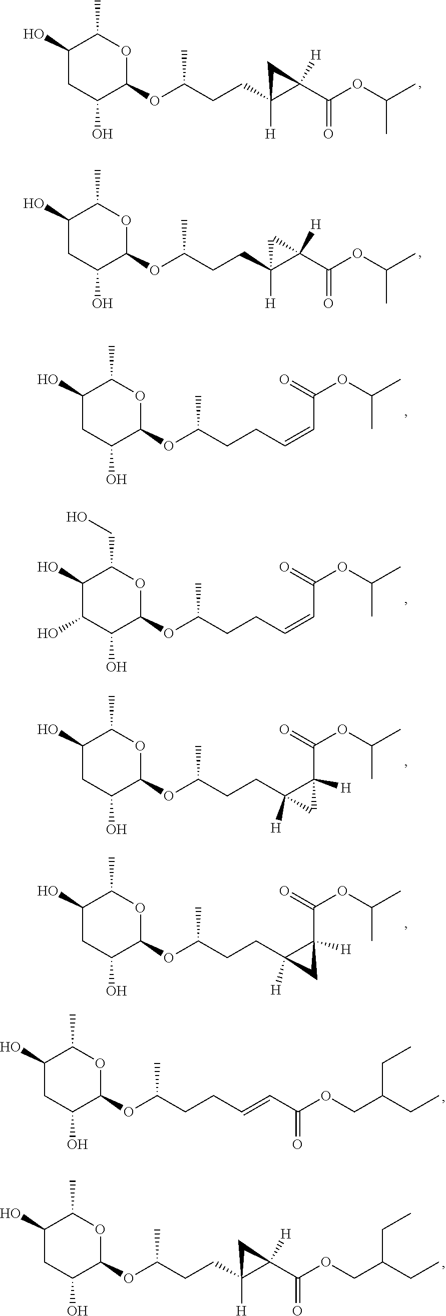

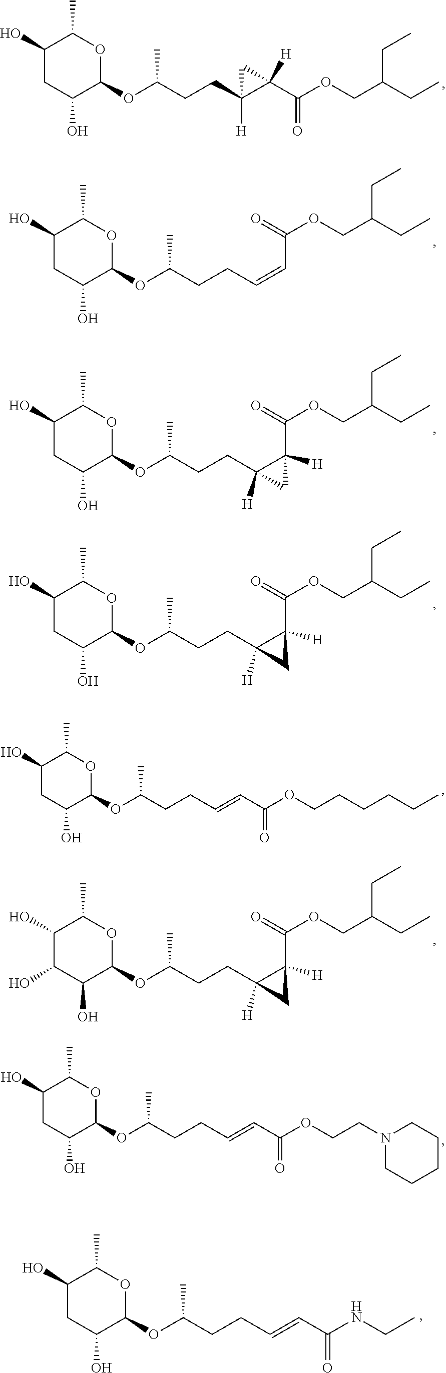

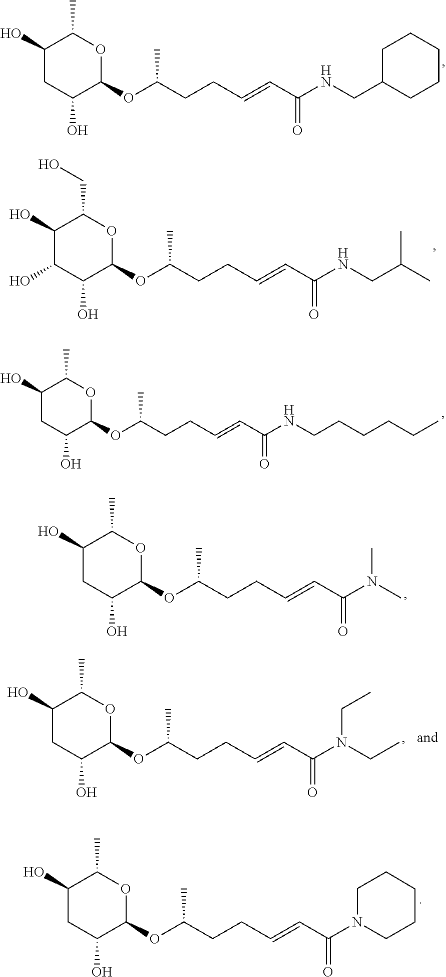

In some embodiments, the compound of Formula III is selected from

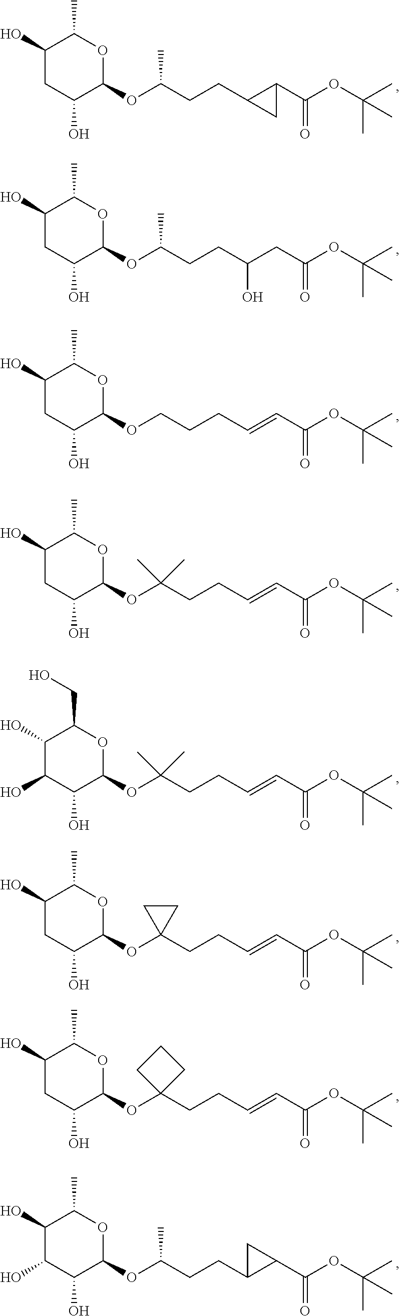

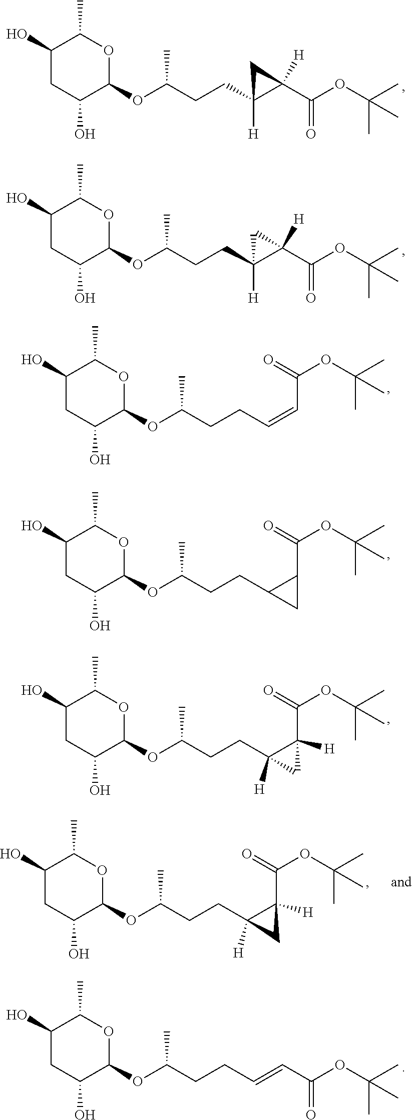

In one aspect, provided herein is a compound of Formula IV with the following structural formula:

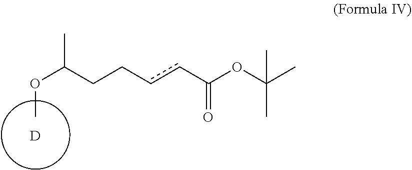

wherein Ring D is selected from:

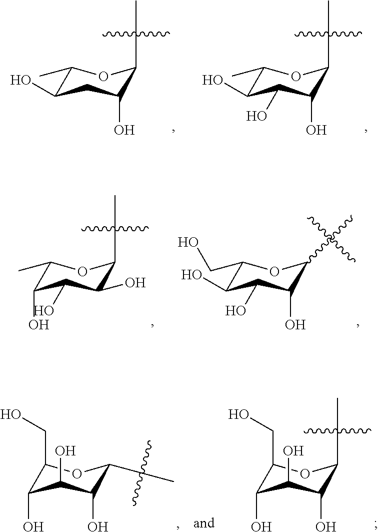

represents the point of attachment of Ring D to the oxygen atom; and [symbol]
is a double bond or a single bond.

In some embodiments, the compound of Formula IV is the compound with the following structural formula:

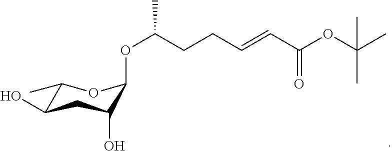

In some embodiments, the compound of Formula IV is the compound with the following structural formula:

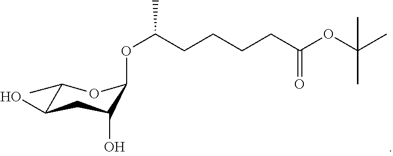

In some embodiments, the compound of Formula IV is selected from

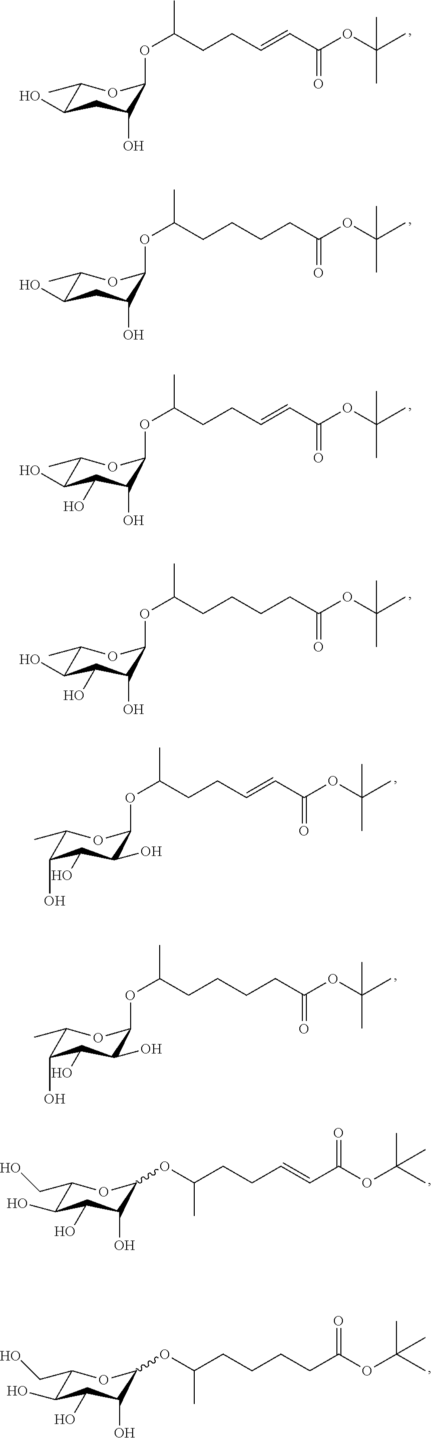

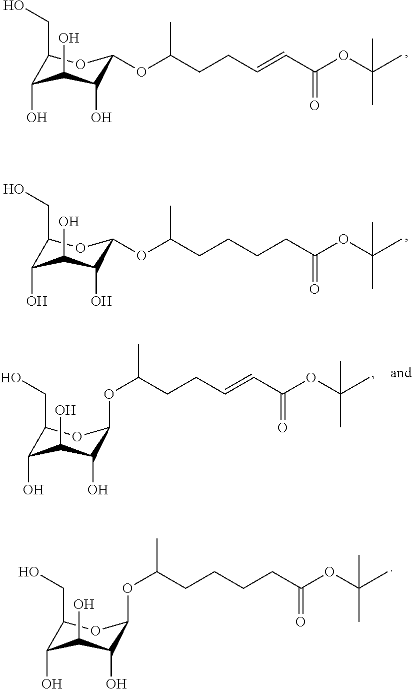

In certain embodiments, Ring D is

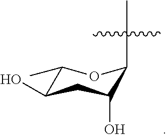

In certain embodiments, Ring D is

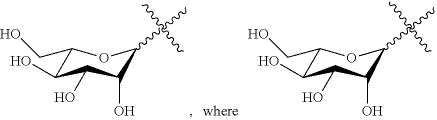

represents a mixture of OH and

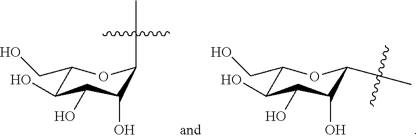

In certain embodiments, Ring D is

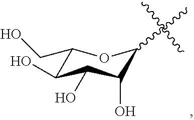

where

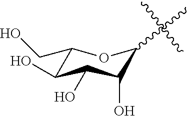

represents

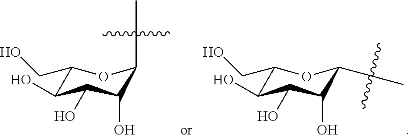

In some embodiments, [symbol] is a double bond.

In some embodiments, the stereochemistry at the methyl-substituted carbon is R.

In some embodiments, [symbol] is a double bond; and the stereochemistry at the methyl-substituted carbon is R.

In another aspect, provided herein are for selectively altering a subject's gut microbiota in a subject in need thereof administering to the subject a composition comprising pharmaceutically-acceptable carrier and an effective amount of the compound of having the structure of Formula III:

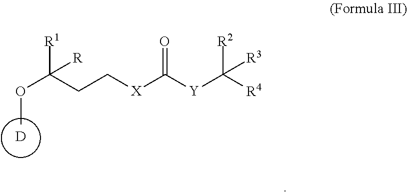

wherein Ring D is selected from:

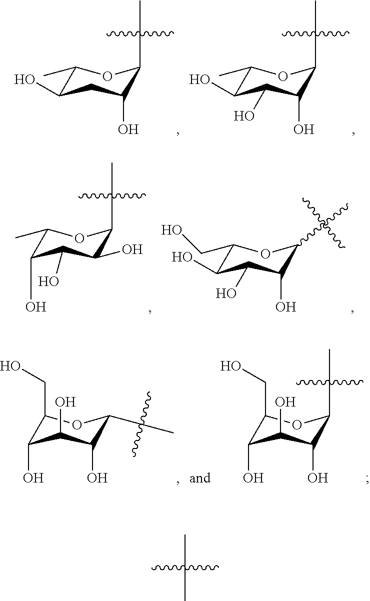

represents the point of attachment of Ring D to the oxygen atom;
X is —C(R⁵)₂—C(R⁵)₂— or E or Z —CH═CH—;

### Y is O or NR⁶;

### R is H or CH₃,

R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring;
thereby selectively modifying the composition of the gut microbiota.

In some embodiments of the methods disclosed herein, the compound of Formula III is selected from:

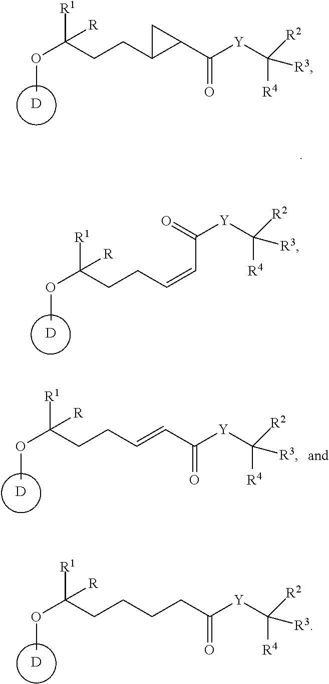

In some embodiments of the methods disclosed herein, X is —C(R⁵)₂—C(R⁵)₂—. In some embodiments, X is E or Z —CH═CH—. In some embodiments, X is —C(H)₂—C(H)₂—. In some embodiments, X is —C(H)(OH)—C(H)₂—.

In some embodiments, X —C(R⁵)₂—C(R⁵)₂, two instances of R⁵, taken together with the carbon atom or carbon atoms to which they are bonded, form a 3-membered carbocyclic ring.

In some embodiments, X is

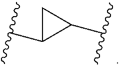 [SMILES: *C1CC1*]

In some embodiments, X is

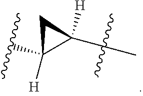 [SMILES: *[C@@H]1C[C@H]1*]

In some embodiments, X is

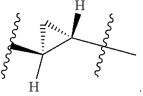 [SMILES: *[C@H]1C[C@@H]1*]

In some embodiments, X is

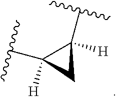 [SMILES: *[C@@H]1C[C@@H]1*]

In some embodiments, X is

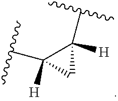 [SMILES: *[C@@H]1C[C@@H]1*]

In some embodiments of the methods disclosed herein, Y is NR⁶. In some embodiments, Y is NR⁶, wherein R⁶ is H. In some embodiments, Y is NR⁶, wherein R⁶ is C₁-C₆ alkyl. In some embodiments, Y is NR⁶, wherein R⁶ is CH₃. In some embodiments, Y is NR⁶, wherein R⁶ is CH₂CH₃. In some embodiments, Y is NR⁶, wherein R⁴ and NR⁶, taken together with the carbon atom to which they are bonded, form a 5- or 6-membered heterocyclic ring. In some embodiments, Y is O.

In some embodiments of the methods disclosed herein, R and R¹ are each CH₃. In some embodiments, R and R¹ are each CH₃. In some embodiments, R is CH₃; and R¹ is H. In some embodiments, R and R¹, taken together with the carbon atom to which they are bonded, form a cyclopropyl. In some embodiments, R and R¹, taken together with the carbon atom to which they are bonded, form a cyclobutyl.

In some embodiments of the methods disclosed herein, R² is H; R³ is H or CH₃; and R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl, or, when Y is NR⁶, R⁴ and NR⁶, taken together with the carbon atom to which they are bonded, form a 5- or 6-membered heterocyclic ring. In some embodiments, R², R³ and R⁴ are each CH₃. In some embodiments, R² is H; R³ is CH₃; and R⁴ is CH₃. In some embodiments, R² is H; R³ is H; and R⁴ is CH₂CH₃. In some embodiments, R² is H; R³ is H; and R⁴ is CH(CH₂CH₃)₂. In some embodiments, R² is H; R³ is H; and R⁴ is CH(CH₂CH₃)₂. In some embodiments, R² is H; R³ is H; and R⁴ is cyclohexyl. In some embodiments, R² is H; R³ is H; and R⁴ is phenyl. In some embodiments, R² is H; R³ is H; and R⁴ is pyridinyl.

In another aspect, provided herein are methods selectively altering a subject's gut microbiota, in a subject in need thereof comprising administering to the to the subject's gastrointestinal system a composition comprising pharmaceutically-acceptable carrier and an effective amount of Ascaroside derivative having the structure of Formula IV

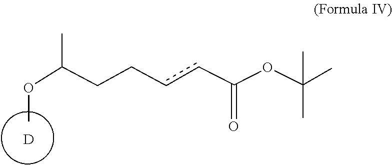

Wherein Ring D is selected from:

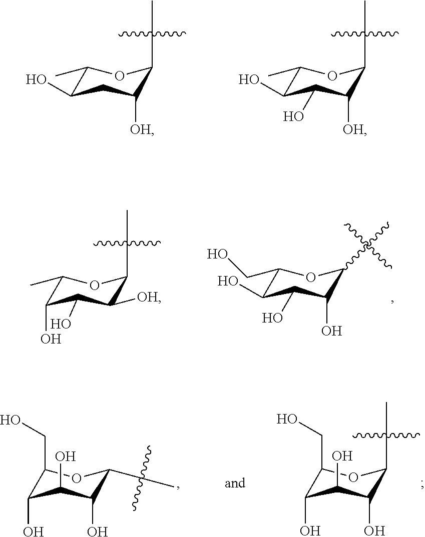

represents the point of attachment of Ring D to the oxygen atom; and [symbol]
is a double bond or a single bond; thereby selectively modifying the composition of the gut microbiota to increase one or more of: the diversity of the gut microbiota, the amount of Bifidobacterium, the amount of Akkermansia and the amount of Adlercreutzia within the subject's gastrointestinal system.

In some embodiments of the methods disclosed herein, the compound of Formula IV is the compound having the structure:

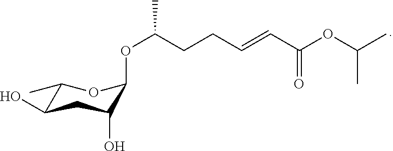

In some embodiments of the methods disclosed herein, the compound of Formula IV is the compound having the structure:

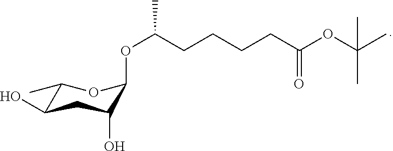

In some embodiments of the methods disclosed herein, the compound of Formula IV is a compound selected from

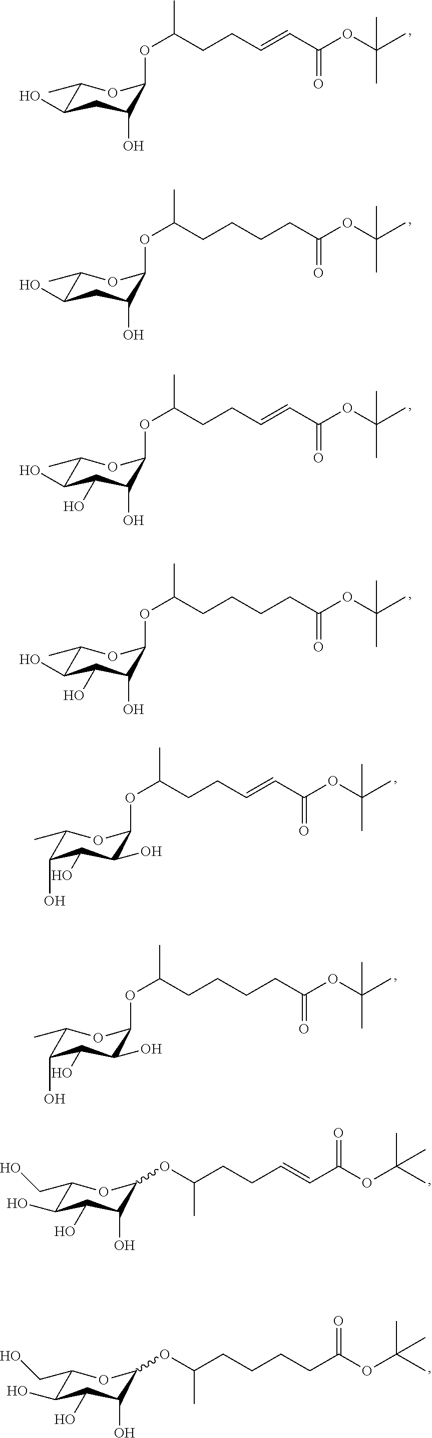

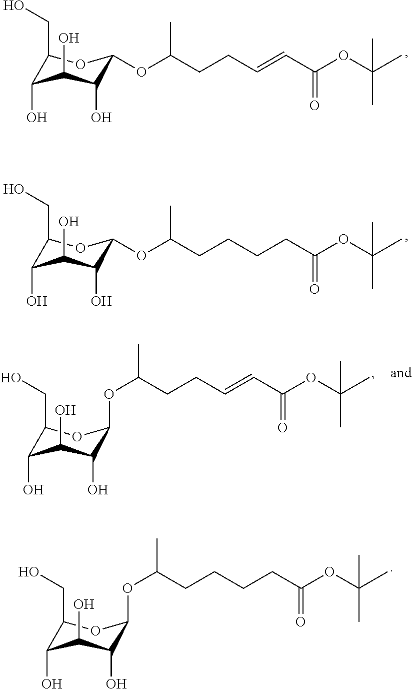

In another aspect, provided herein are methods of increasing the diversity of a subject's gut microbiota, the method comprising: administering to the subject's gastrointestinal system a composition comprising pharmaceutically-acceptable carrier and an effective amount of Ascaroside derivative having the structure of Formula III,

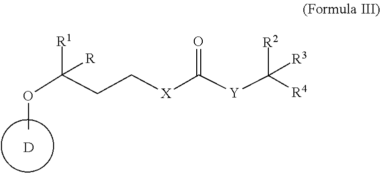

wherein Ring D is selected from:

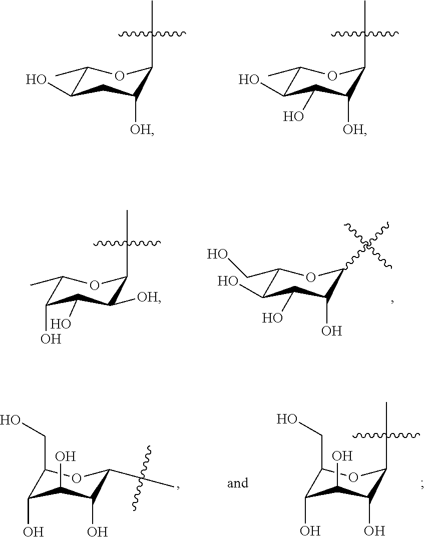

represents the point of attachment of Ring D to the oxygen atom;
X is —C(R⁵)₂—C(R⁵)₂— or E or Z —CH═CH—;

### Y is O or NR⁶;

### R is H or CH₃,

R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring; so that within the subject's intestinal tract, the Ascaroside derivative modifies the diversity of the subject's gut microbiota.
In some embodiments of the methods disclosed herein, the compound of Formula III is selected from:

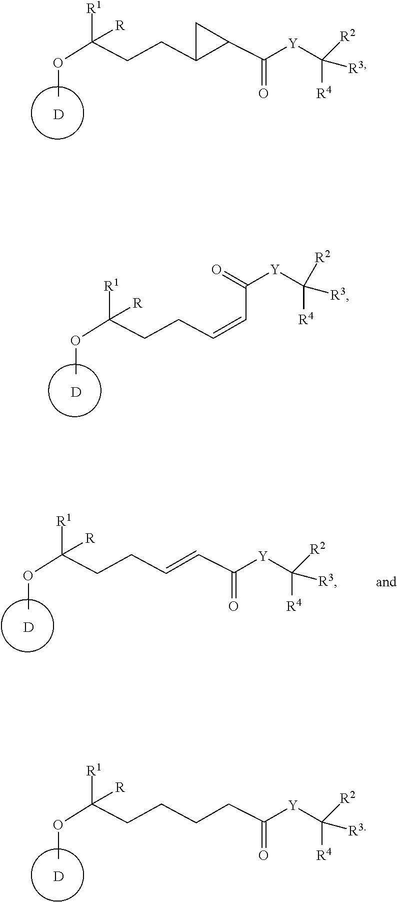

In some embodiments of the methods disclosed herein, X is —C(R⁵)₂—C(R⁵)₂—. In some embodiments, X is E or Z —CH═CH—. In some embodiments, X is —C(H)₂—C(H)₂—. In some embodiments, X is —C(H)(OH)—C(H)₂—.

In some embodiments, X —C(R⁵)₂—C(R⁵)₂, two instances of R⁵, taken together with the carbon atom or carbon atoms to which they are bonded, form a 3-membered carbocyclic ring.

In some embodiments, X is

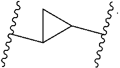 [SMILES: *C1CC1*]

In some embodiments, X is

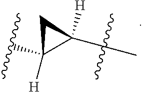 [SMILES: *[C@@H]1C[C@H]1*]

In some embodiments, X is

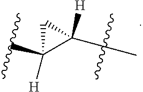 [SMILES: *[C@H]1C[C@@H]1*]

In some embodiments, X is

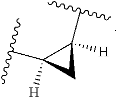 [SMILES: *[C@@H]1C[C@@H]1*]

In some embodiments, X is

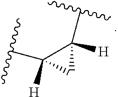 [SMILES: *[C@@H]1C[C@@H]1*]

In some embodiments of the methods disclosed herein, Y is NR⁶.

In some embodiments, Y is NR⁶, wherein R⁶ is H. In some embodiments, Y is NR⁶, wherein R⁶ is C₁-C₆ alkyl. In some embodiments, Y is NR⁶, wherein R⁶ is CH₃. In some embodiments, Y is NR⁶, wherein R⁶ is CH₂CH₃. In some embodiments, Y is NR⁶, wherein R⁴ and NR⁶, taken together with the carbon atom to which they are bonded, form a 5- or 6-membered heterocyclic ring. In some embodiments, Y is O.

In some embodiments of the methods disclosed herein, R and R¹ are each CH₃. In some embodiments, R and R¹ are each CH₃. In some embodiments, R is CH₃; and R¹ is H. In some embodiments, R and R¹, taken together with the carbon atom to which they are bonded, form a cyclopropyl. In some embodiments, R and R¹, taken together with the carbon atom to which they are bonded, form a cyclobutyl.

In some embodiments of the methods disclosed herein, R² is H; R³ is H or CH₃; and R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl, or, when Y is NR⁶, R⁴ and NR⁶, taken together with the carbon atom to which they are bonded, form a 5- or 6-membered heterocyclic ring. In some embodiments, R², R³ and R⁴ are each CH₃. In some embodiments, R² is H; R³ is CH₃; and R⁴ is CH₃. In some embodiments, R² is H; R³ is H; and R⁴ is CH₂CH₃. In some embodiments, R² is H; R³ is H; and R⁴ is CH(CH₂CH₃)₂. In some embodiments, R² is H; R³ is H; and R⁴ is CH(CH₂CH₃)₂. In some embodiments, R² is H; R³ is H; and R⁴ is cyclohexyl. In some embodiments, R² is H; R³ is H; and R⁴ is phenyl. In some embodiments, R² is H; R³ is H; and R⁴ is pyridinyl.

In another aspect, provided herein are methods of treating constipation in a subject, the method comprising administering to the subject's gastrointestinal system a composition comprising pharmaceutically-acceptable carrier and an effective amount of the compound having the structure of Formula IV:

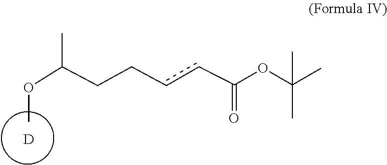

Wherein Ring D is selected from

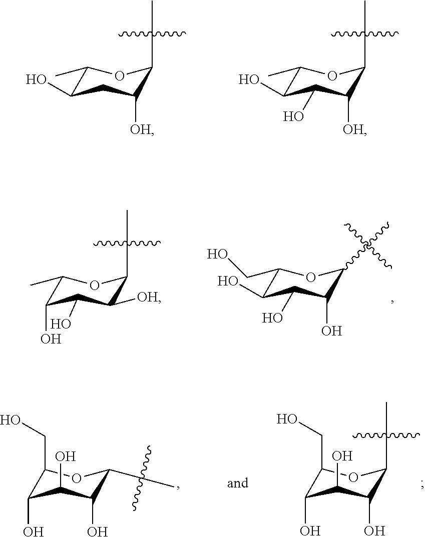

represents the point of attachment of Ring D to the oxygen atom; and

[symbol] is a double bond or a single bond; and fecal softness is increased.

In certain embodiments, the composition is administered orally. In other embodiments, the composition is administered rectally (e.g., by suppository). In some variations the compositions is administered by direct injection, topically, and/or intravenously.

In any of the methods described herein, the subject may be selected from primates, humans, equines, horses, cattle, cows, swine, sheep, rodents, rats, pets, dogs, and guinea pigs. In certain preferred embodiments, the subject is a human. In other embodiments, the subject is a rodent. In yet other embodiments, the subject is selected from primates, equines, horses, cattle, cows, swine, sheep, rats, pets, cats, dogs and guinea pigs. In some embodiments, the subject is a female. In other embodiments, the subject is a male. In some embodiments, the subject is an infant, a child, or an adult.

In another aspect, provided herein are uses of the compound having the structure of Formula III in the manufacture of a medicament for increasing the diversity of a subject's gut microbiota, and/or for treating constipation in a subject. In some embodiments of the uses, the compound is compound 1. In some embodiments of the uses, the compound is compound 10.

In some embodiments of any of the methods disclosed herein, the compound of Formula III is selected from:

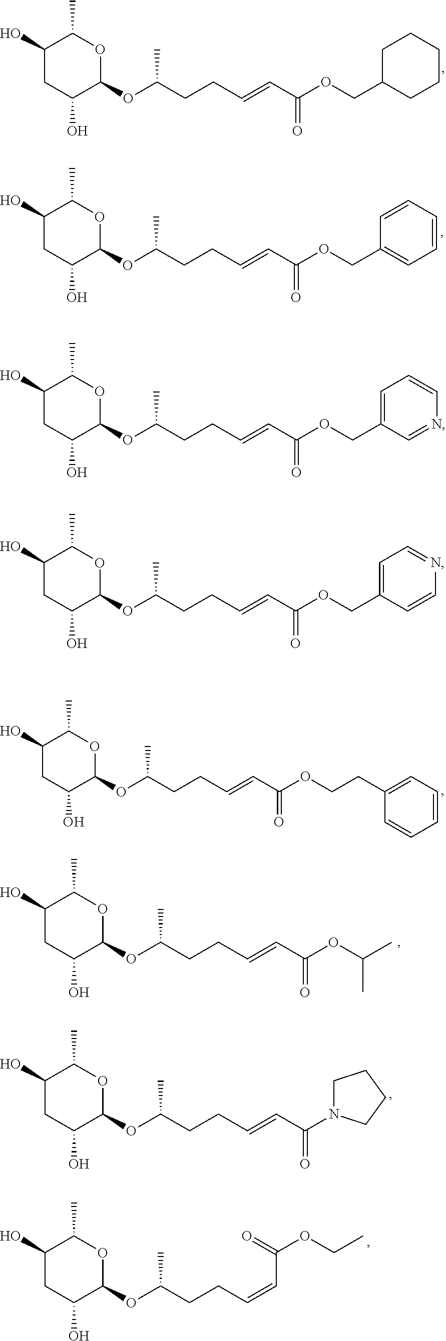

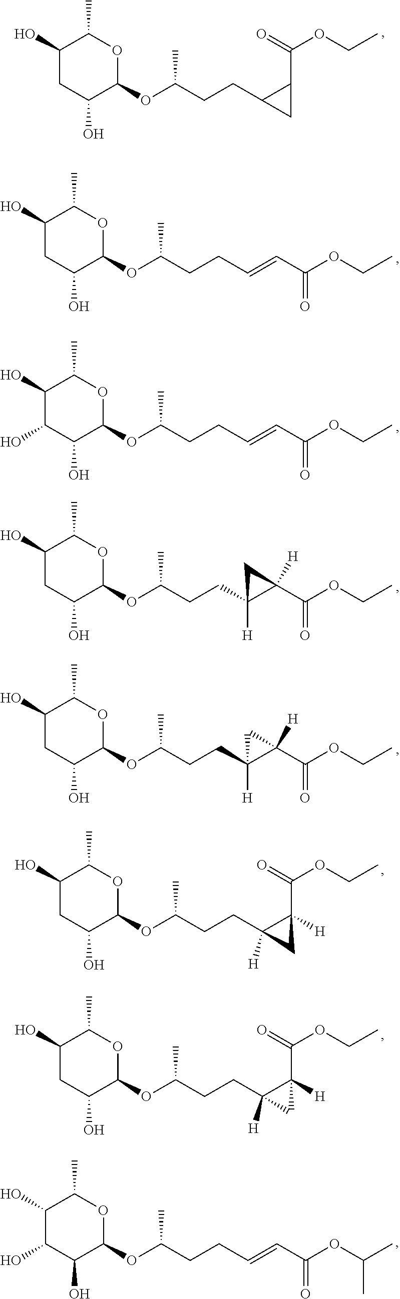

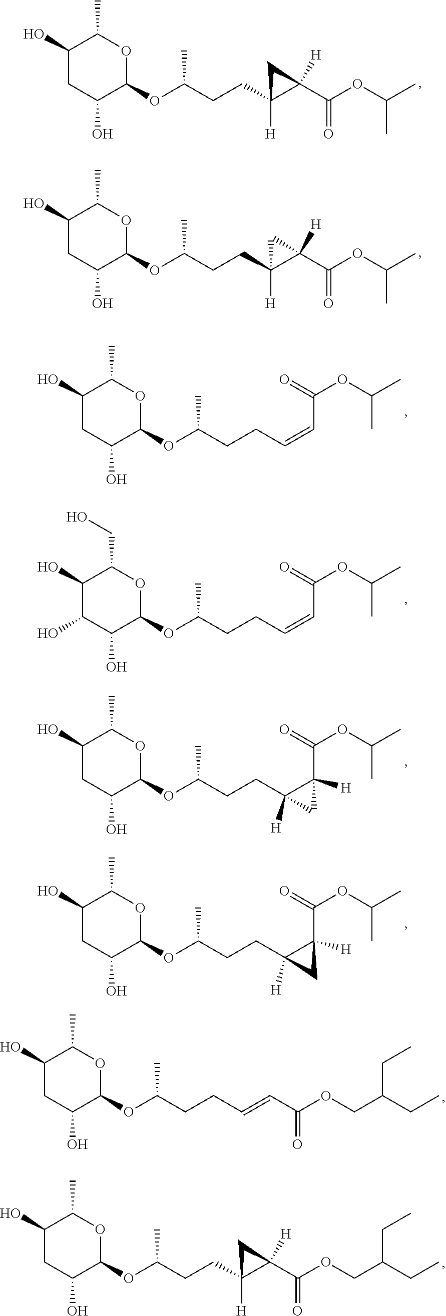

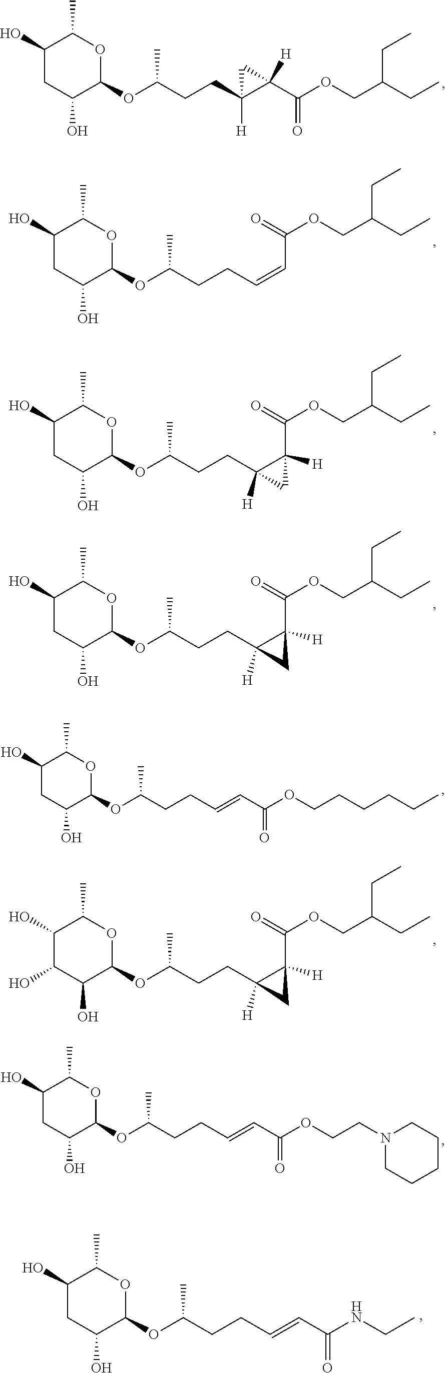

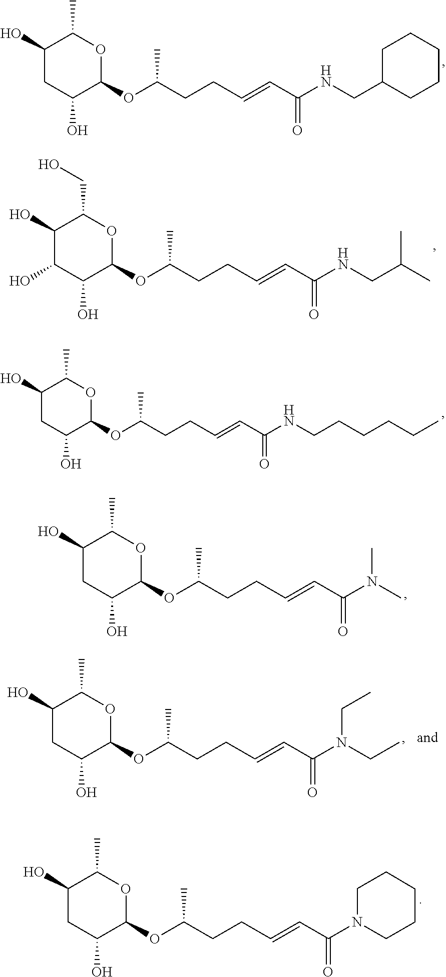

In some embodiments of any of the methods disclosed herein, the compound of Formula III is selected from:

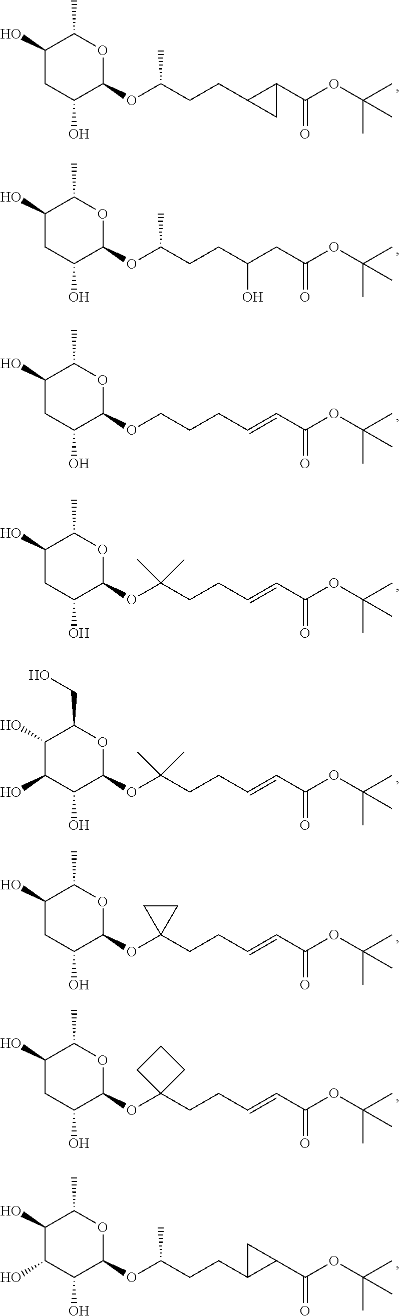

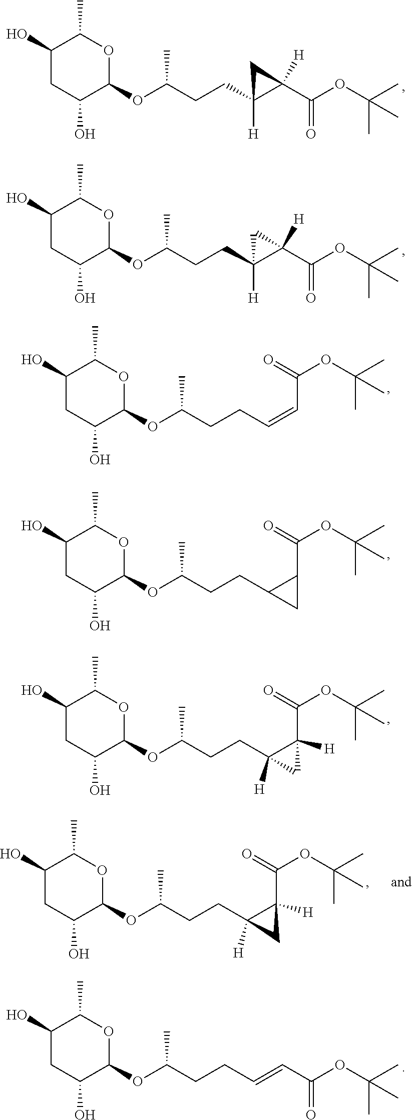

In some embodiments of any of the methods, the compound of Formula IV is selected from:

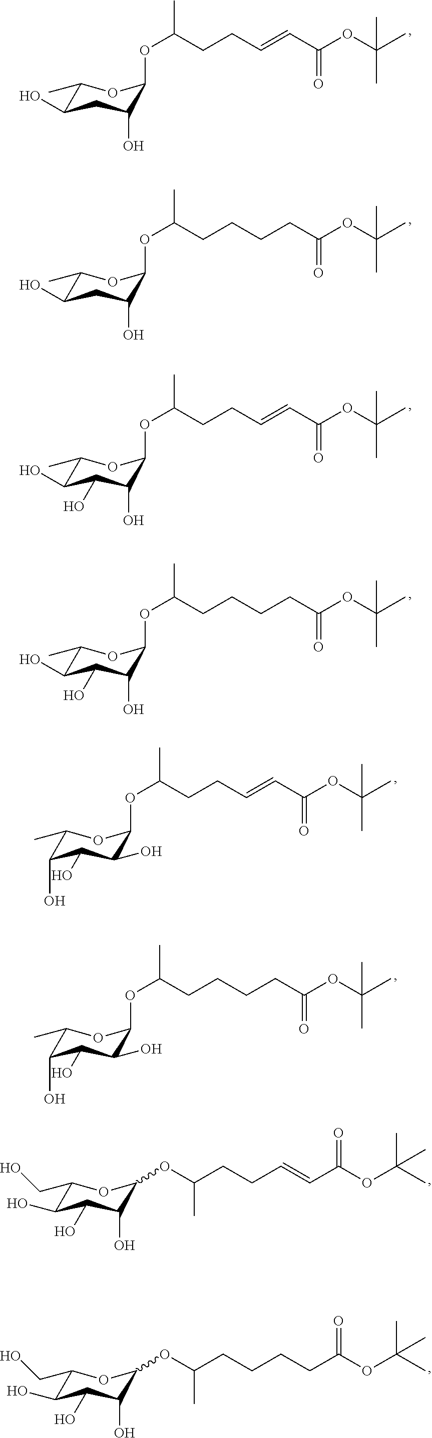

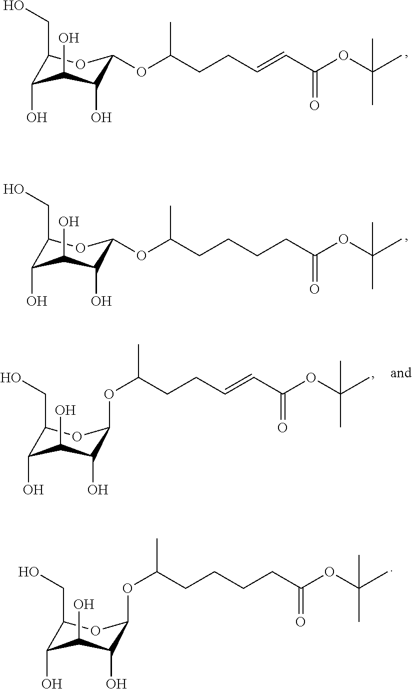

In another aspect, provided herein are pharmaceutical formulations comprising a pharmaceutically acceptable carrier; and the compound having the structure of Formula III:

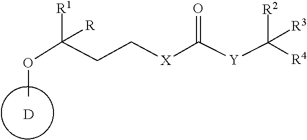

Formula III

wherein

Ring D is selected from

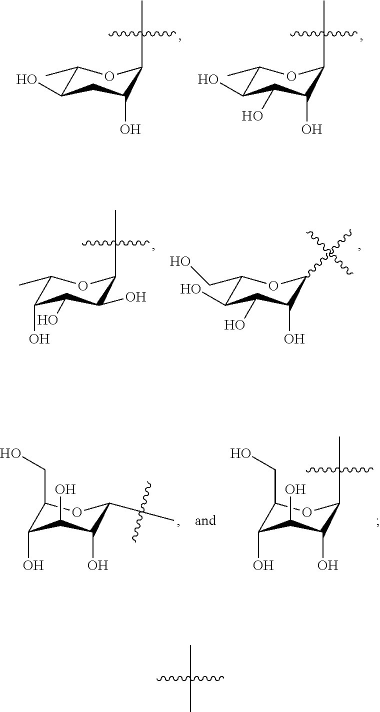

represents the point of attachment of Ring D to the oxygen atom;

X is —C(R⁵)₂—C(R⁵)₂— or E or Z —CH═CH—;

Y is O or NR⁶;

R is H or CH₃,

R¹ is H or CH₃;

or, R and R¹, taken together with the carbon atom to which they are bonded, form a 3- or 4-membered carbocyclic ring;

R² is H or CH₃;

R³ is H or CH₃;

R⁴ is CH₃, CH₂CH₃, a straight- or branched-chain C₃-C₆ alkyl, a C₅-C₇ cycloalkyl,

a 6-membered aryl or heteroaryl, an aryl-substituted C₁-C₆ alkyl, or a heterocyclyl-substituted C₁-C₆ alkyl;

R⁵, independently for each occurrence, is H or OH,

or two instances of R⁵, taken together with the carbon atom or carbon atoms to which they are bonded, form a 3-membered carbocyclic ring; and

R⁶ is H or C₁-C₆ alkyl,

or, when Y is NR⁶, R⁴ and NR⁶, taken together with the carbon atom to which they are bonded, form a 5- or 6-membered heterocyclic ring.

In some embodiments of the pharmaceutical formulations, X is —C(R⁵)₂—C(R⁵)₂—. In some embodiments, X is E or Z —CH═CH—. In some embodiments, X is —C(H)₂—C(H)₂—. In some embodiments, X is —C(H)(OH)—C(H)₂—.

In some embodiments, X —C(R⁵)₂—C(R⁵)₂, two instances of R⁵, taken together with the carbon atom or carbon atoms to which they are bonded, form a 3-membered carbocyclic ring. In some embodiments, X is

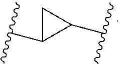 [SMILES: *C1CC1*]

In some embodiments, X is

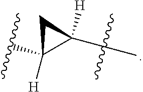 [SMILES: *[C@@H]1C[C@H]1*]

In some embodiments, X is

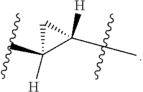 [SMILES: *[C@H]1C[C@@H]1*]

In some embodiments, X is

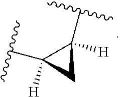 [SMILES: *[C@@H]1C[C@@H]1*]

In some embodiments, X is

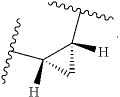 [SMILES: *[C@@H]1C[C@@H]1*]

In some embodiments of the pharmaceutical formulations, Y is NR⁶. In some embodiments, Y is NR⁶, wherein R⁶ is H. In some embodiments, Y is NR⁶, wherein R⁶ is C₁-C₆ alkyl. In some embodiments, Y is NR⁶, wherein R⁶ is CH₃. In some embodiments, Y is NR⁶, wherein R⁶ is CH₂CH₃. In some embodiments, Y is NR⁶, wherein R⁴ and NR⁶, taken together with the carbon atom to which they are bonded, form a 5- or 6-membered heterocyclic ring. In some embodiments, Y is O.

In some embodiments of the pharmaceutical formulations, R and R¹ are each CH₃. In some embodiments, R and R¹ are each CH₃. In some embodiments, R is CH₃; and R¹ is H. In some embodiments, R and R¹, taken together with the carbon atom to which they are bonded, form a cyclopropyl. In some embodiments, R and R¹, taken together with the carbon atom to which they are bonded, form a cyclobutyl.

In some embodiments of the pharmaceutical formulations, R² is H; R³ is H or CH₃; and R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl, or, when Y is NR⁶, R⁴ and NR⁶, taken together with the carbon atom to which they are bonded, form a 5- or 6-membered heterocyclic ring. In some embodiments, R², R³ and R⁴ are each CH₃. In some embodiments, R² is H; R³ is CH₃; and R⁴ is CH₃. In some embodiments, R² is H; R³ is H; and R⁴ is CH₂CH₃. In some embodiments, R² is H; R³ is H; and R⁴ is CH(CH₂CH₃)₂. In some embodiments, R² is H; R³ is H; and R⁴ is CH(CH₂CH₃)₂. In some embodiments, R² is H; R³ is H; and R⁴ is cyclohexyl. In some embodiments, R² is H; R³ is H; and R⁴ is phenyl. In some embodiments, R² is H; R³ is H; and R⁴ is pyridinyl.

In some embodiments of the pharmaceutical formulations, the compound of Formula III is selected from:

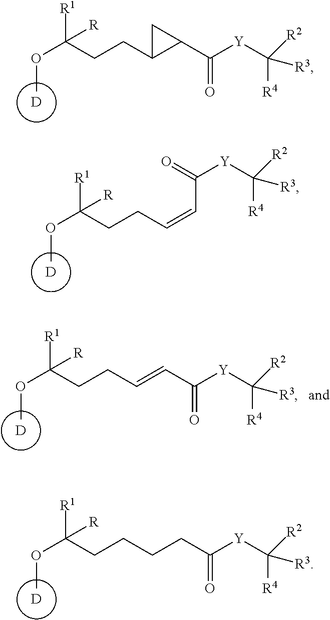

In some embodiments of the pharmaceutical formulations, the compound of Formula III is selected from:

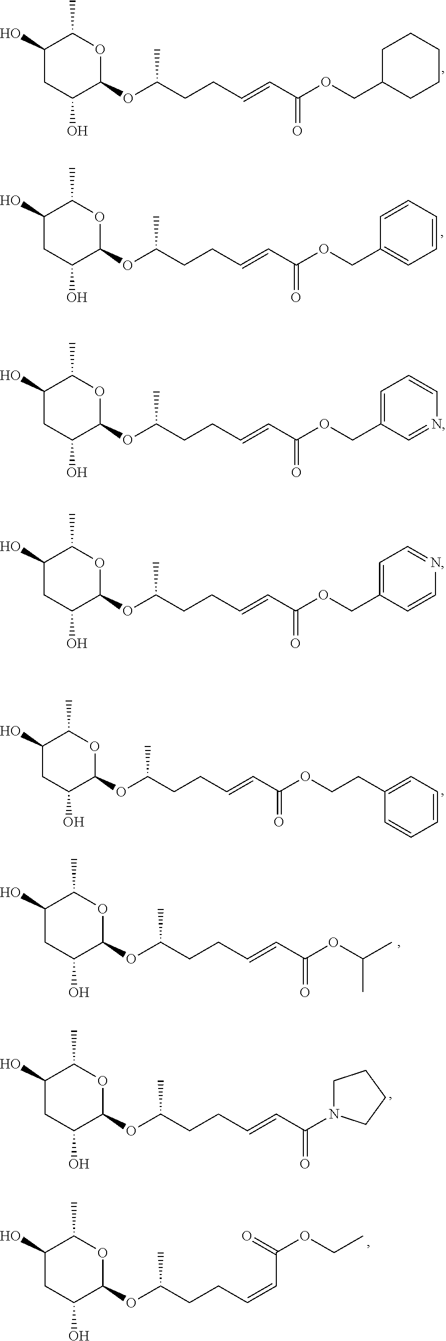

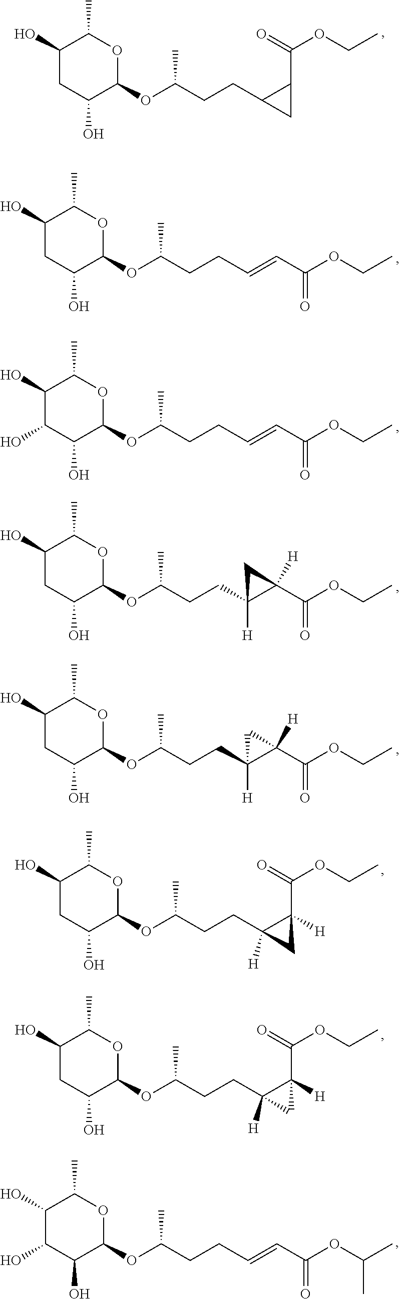

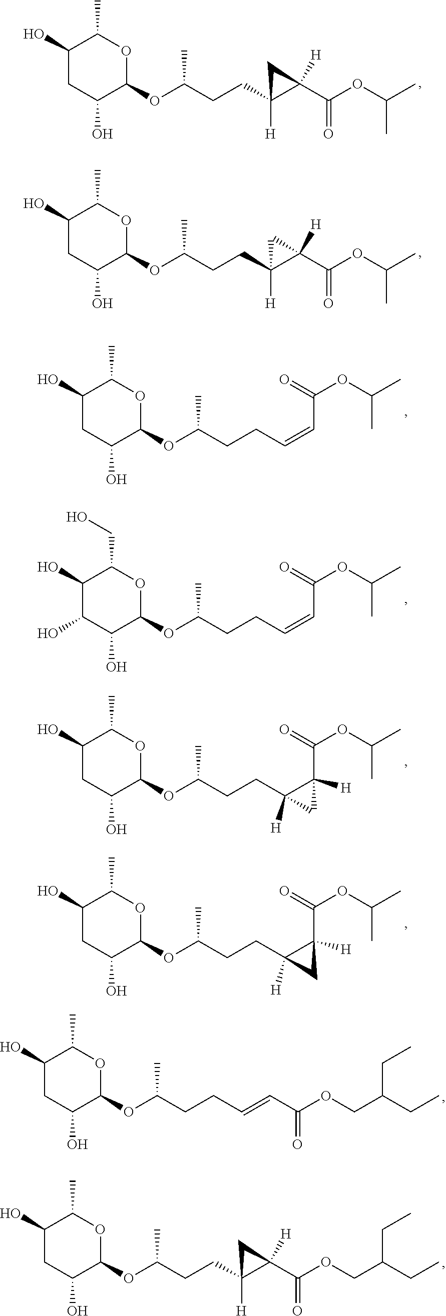

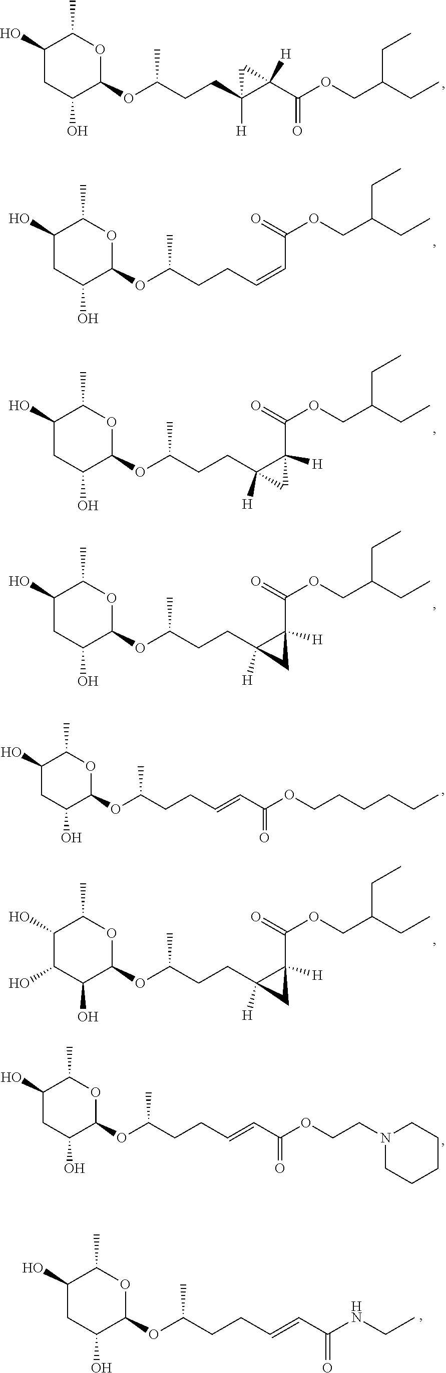

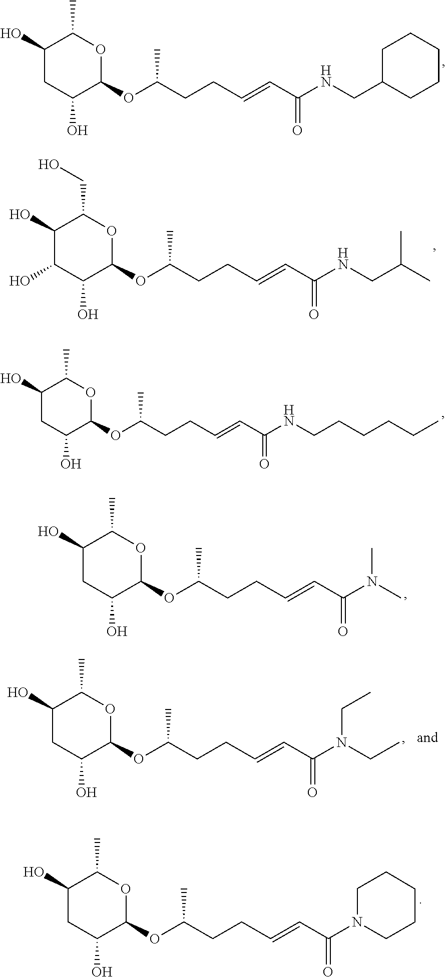

In some embodiments of the pharmaceutical formulations, the compound of Formula III is selected from:

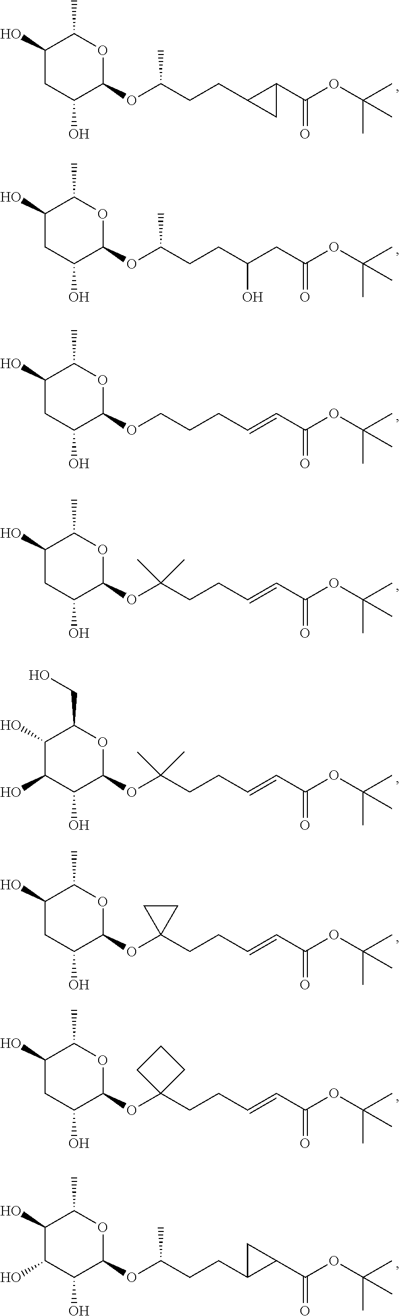

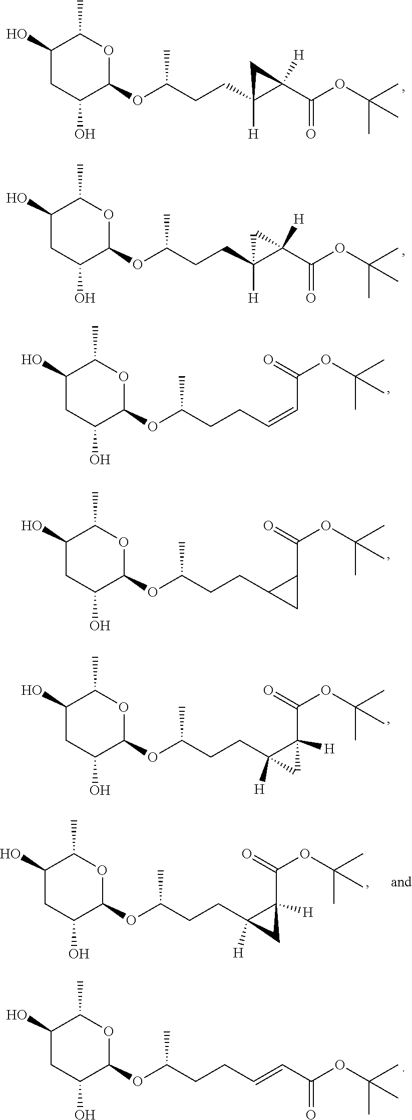

In another aspect, provided herein are pharmaceutical formulations comprising a pharmaceutically acceptable carrier; and the compound having the structure of Formula IV:

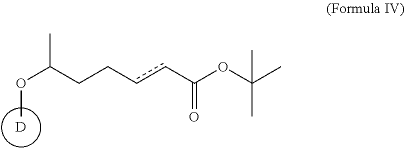

wherein Ring D is selected from:

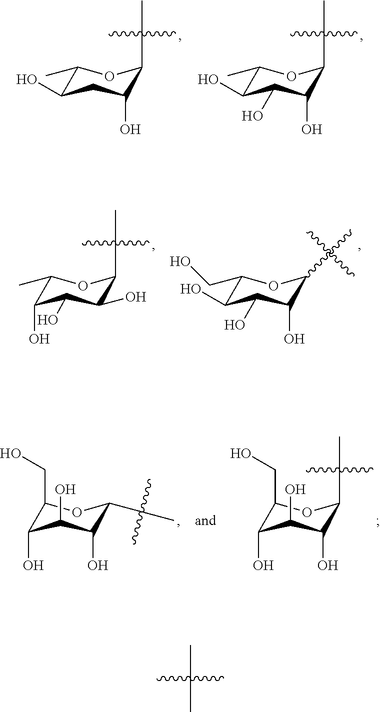

represents the point of attachment of Ring D to the oxygen atom; and [symbol]
is a double bond or a single bond.

In some embodiments of the pharmaceutical formulations, the compound of Formula IV is a compound with the following structural formula:

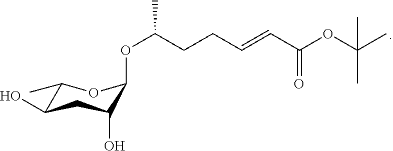

In some embodiments of the pharmaceutical formulations, the compound of Formula IV is a compound with the following structural formula:

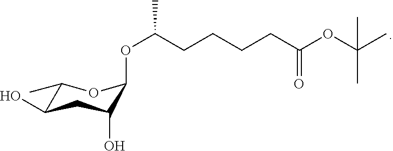

In some embodiments of the pharmaceutical formulations, the compound of Formula IV is selected from:

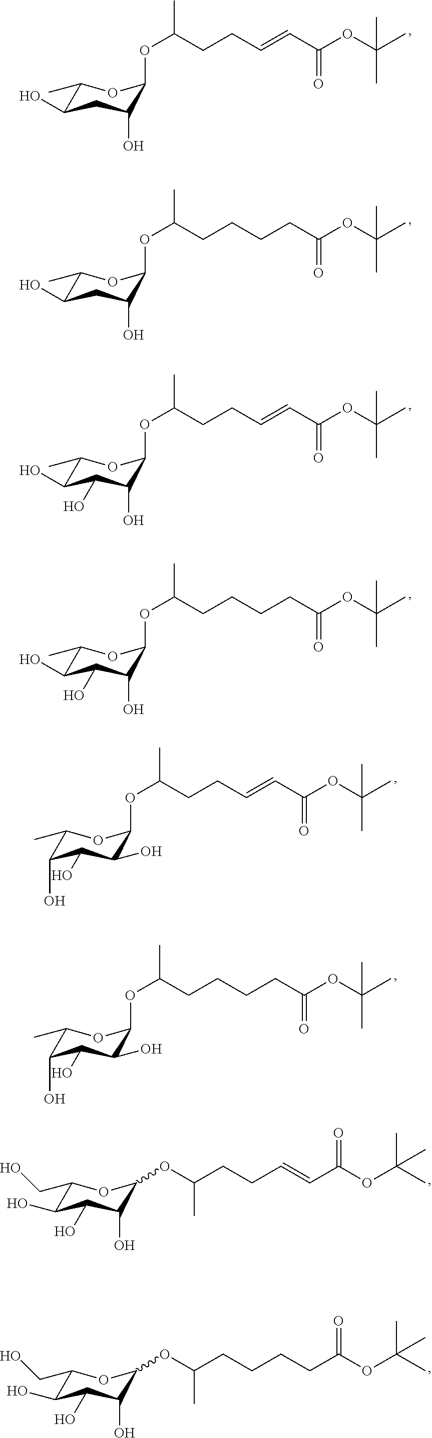

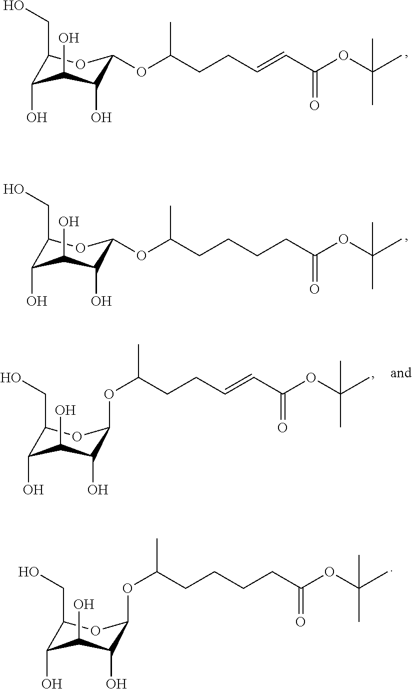

The compounds, compositions and methods described herein may be utilized to treat a subject in need thereof. In certain embodiments, the subject is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop

A pharmaceutical composition (preparation, formulation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,731,000, 5,541,231, 5,427,798, 5,358,970 and 4,172,896, as well as in patents cited therein.

One of skill in the art would appreciate that a method of administering a formulation or composition of the disclosure would depend on factors such as the age, weight, and physical condition of the subject being treated, and the disease or condition being treated. The skilled worker would, thus, be able to select a method of administration optimal for a subject on a case-by-case basis.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for administration to the mouth may be presented as a mouthwash, or an oral spray, or an oral ointment.

Alternatively, or additionally, compositions can be formulated for delivery via a catheter, stent, wire, or other intraluminal device. Delivery via such devices may be especially useful for delivery to the bladder, urethra, ureter, rectum, or intestine.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The subject receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Ascaroside Derivative Examples

### Synthesis of Compound 1

The compound was synthesized according to the following scheme.

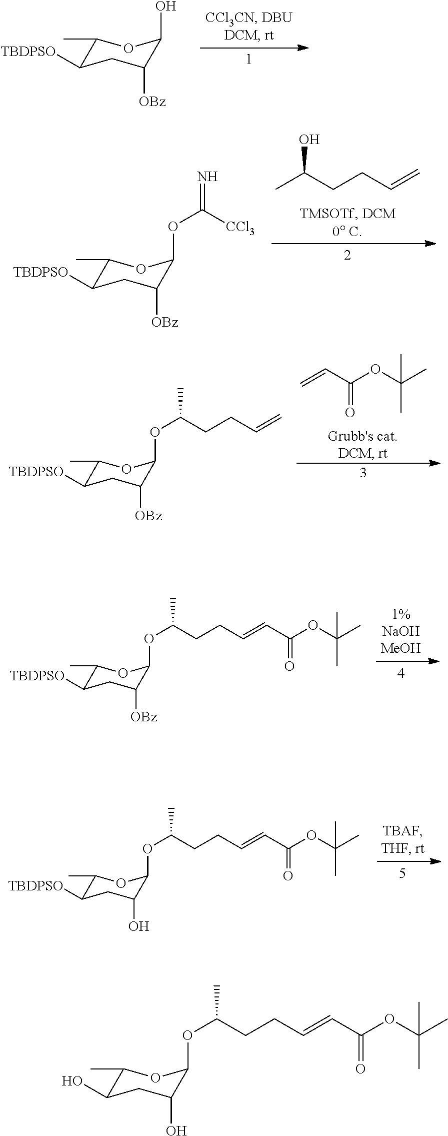

### Synthesis of Compound 10

The compound was synthesized according to the following scheme.

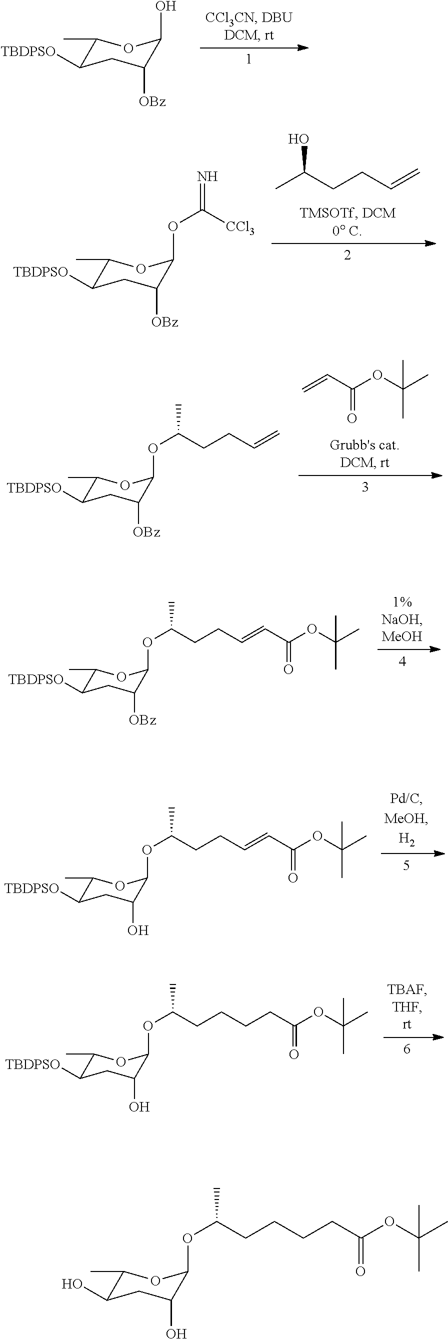

I. Generic Synthesis

Ascaroside derivative compounds were prepared using methods illustrated in the general synthetic schemes and experimental procedures detailed below. These general synthetic schemes and experimental procedures are presented for purposes of illustration and are not intended to be limiting. Starting materials used to prepare compounds of the present invention are commercially available or can be prepared using routine methods known in the art. Where present, names of compounds were generated using ChemAxon's Instant JChem v6.1 for Desktop and IUPAC Naming Plugin or ChemDraw Professional 16.0.1.4 (61).

General methods for the synthesis of ascaroside pheromones and related compounds have been previously described in the literature, e.g. Jeong P. Y. et al. Chemical structure and biological activity of the Caenorhabditis elegans dauer-inducing pheromone. Nature 2005, 433 (7025), 541-545; Martin, R. et al. Improved Synthesis of an Ascaroside Pheromone Controlling Dauer Larva Development in Caenorhabditis elegans. Synthesis 2009, 20, 3488-3492; and Zhang, Y. K. et al. Improved Synthesis for Modular Ascarosides Uncovers Biological Activity. Org. Lett. 2017, 19, 2837-2840, each of which are hereby incorporated by reference.

Protected carbohydrates (I) with a free anomeric hydroxyl group are activated by making O-glycosyl trichloroacetimidates IV, which are prepared using trichloroacetonitrile (Cl₃CCN) under basic conditions (e.g. DBU, DIPEA, etc.). Scheme 1 illustrated the reaction of 0-glycosyl trichloroacetimidates IV with a Lewis acid, such as BF₃.OEt₂ or TMSOTf, in the presence of an alcohol to afford O-glycosylated products (III).

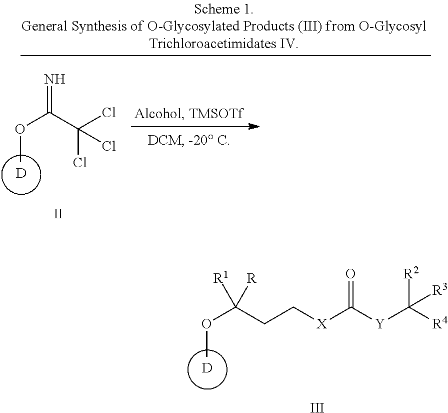

Scheme 2 illustrates the reaction of O-glycosylated terminal olefins (IV) with suitable α,β-unsaturated esters or amides using Grubb's olefin metastasis catalysis (e.g. dichloro[1,3-bis(2,6-isopropylphenyl)-2-imidazolidinylidene](benzylidene) (tricyclohexylphosphine) rutheniumIV, (1,3-bis(2,4,6-trimethylphenyl)-2-imidazolidinylidene)dichloro(phenylmethylene) (tricyclohexylphosphine)ruthenium, benzylidene-bis(tricyclohexylphosphine)dichlororuthenium, bis(tricyclohexylphosphine) benzylidine ruthenium(IV) dichloride, etc.) to afford O-glycosylated (E)-isomer α,β-unsaturated esters or amides (V).

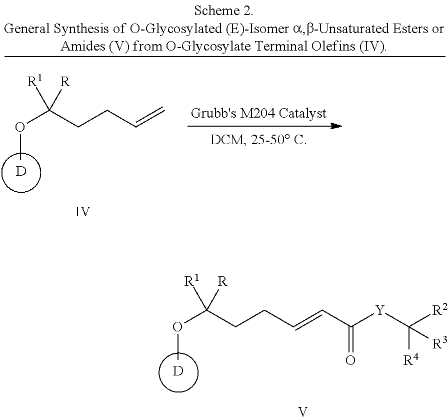

Scheme 3 illustrates the oxidation of O-glycosylated terminal olefins (IV) to afford O-glycosylated aldehydes (VI) using Lemieux oxidation (OsO₄ and NaIO₄) or ozonolysis (O₃) conditions.

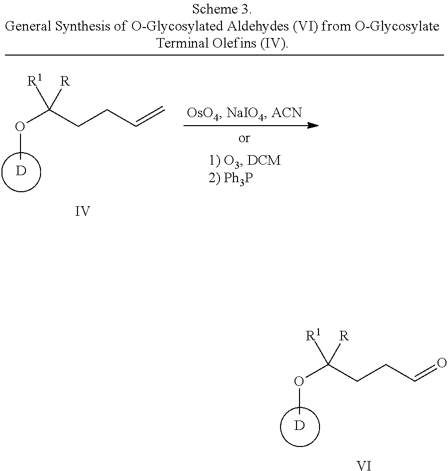

Scheme 4 illustrates the Still-Gennari olefination reaction of O-glycosylated aldehydes (IV) to afford O-glycosylated (Z)-isomer α,β-unsaturated esters or amides (VII). (Z)-Stereospecific Still-Gennari olefination conditions utilize bistrifluoroethylphosphonates (e.g. bis(2,2,2-trifluoroethyl)(methoxycarbonylmethyl)phosphonate), bis(2,2,2-trifluoroethyl) (ethoxycarbonylmethyl) phosphonate), etc.), a strong base such as KHMDS, and a cation savanger, such as 18-crown-6.

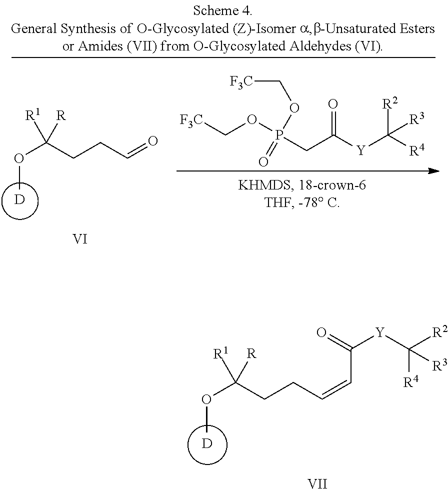

Scheme 5 illustrates the coupling of O-glycosylated carboxylic acids (VIII) with alcohols to form O-glycosylated esters (IX). O-Glycosylated carboxylic acids (VIII) are made by base or acid hydrolysis of simple esters (e.g. methyl, ethyl, tert-butyl) derived from the Grubb's olefin metastasis approach (Scheme 2). Several esterification methods are possible from VIII to IX ketone carbamate esters IV: 1) IIIV is coupled with alcohols (HO—C(R²)(R³)(R⁴)) using a coupling reagent (e.g. N,N′-dicyclohexylcarbodiimide (DCC), EDC, HBTU, HATU, TBTU, PyBOP, etc.) under basic conditions (e.g. triethylamine, diisopropylethylamine, pyridine, N,N-4-dimethylaminopyridine, etc.); 2) IIIV is alkylated with an activated alkane (X—C(R²)(R³)(R⁴)), where X=OTf, OTs, OMs, I, Br, and Cl) under basic conditions (e.g. NaOH, NaH, NaCO₃, K₂CO₃, Cs₂CO₃, NaHCO₃, etc.); and 3) IIIV is esterified with alcohols (HO—C(R²)(R³)(R⁴)) using Mitsunobu conditions (e.g. diethyl azodicarboxylate and triphenylphosine, or similar reagents).

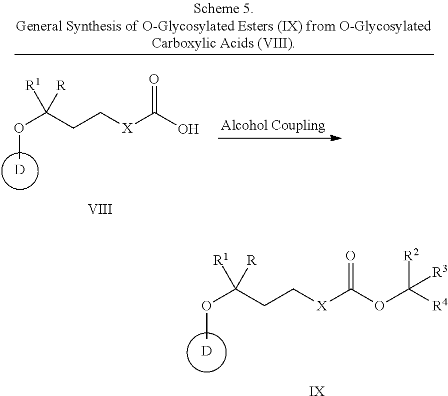

Scheme 6 illustrates the coupling of O-glycosylated carboxylic acids (VIII) with amines to form O-glycosylated amides (X). Standard amide coupling conditions between amines (N(C—(R²)(R³)(R⁴))R⁶) and coupling reagent (e.g. N,N′-dicyclohexylcarbodiimide (DCC), EDC, HBTU, HATU, TBTU, PyBOP, etc.) under basic conditions (e.g. triethylamine, diisopropylethylamine, pyridine, N,N-4-dimethylaminopyridine).

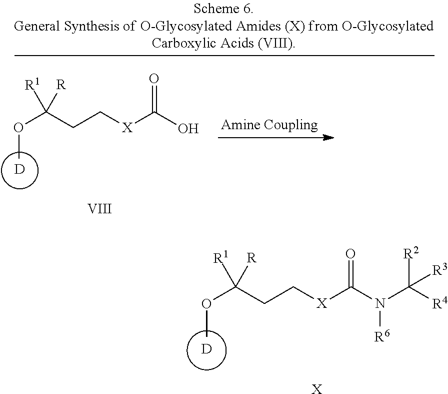

Scheme 7 illustrates the cyclopropanation of O-glycosylated terminal olefins (IV) to form O-glycosylated trans-cyclopropyl esters or amides (XI) under standard diazo cyclopropanation conditions using a rhodium tetracarboxylate catalyst (e.g. rhodiumIV acetate dimer, rhodiumIV trifluoroacetate dimer, tetrakis[N-phthaloyl-(S)-tert-leucinato] dirhodium, dirhodiumIVtetrakis [methyl 2-pyrrolidone-5(S)-carboxylate], etc.).

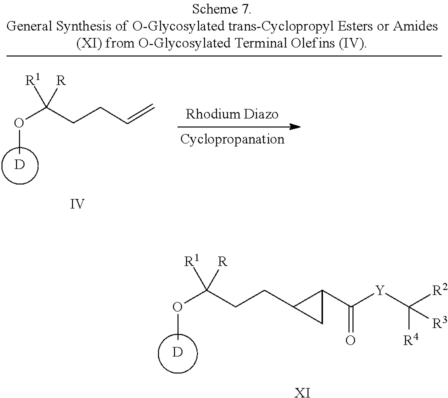

Scheme 8 illustrates the cyclopropanation of O-glycosylated (Z)-isomer α,β-unsaturated esters or amides (VII) to form O-glycosylated cis-cyclopropyl esters or amides (XII). Several methods are possible using Corey-Chaykovsky cyclopropanation conditions (e.g. dimethyloxosulfonium methylide, made using trimethylsulfoxonium iodide in DMSO and sodium hydride) or Simmons-Smith conditions (e.g. iodomethylzinc iodide, made using diiodomethane and zinc-copper couple).

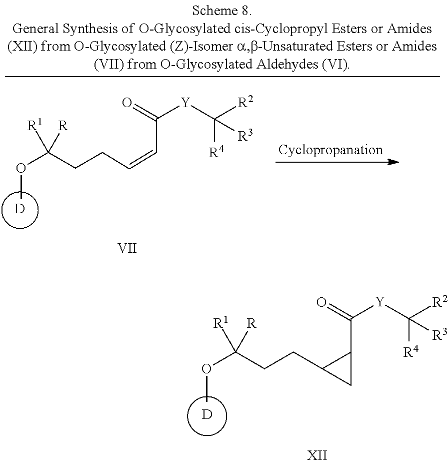

All chemical reagents were purchased commercially and were used without further purification unless otherwise noted. Reactions were done under air/nitrogen atmosphere according to the requirements. Column chromatography was performed on silica gel 60 (230-400 mesh) and analytical TLC was performed on plates coated with silica gel. TLC plates were stained with ceric ammonium molybdate (CAM), p-anisaldehyde (Anis), potassium permanganate (KMnO4), or ninhydrin staining solutions. Routine ¹H NMR spectra were recorded using an Automated Varian Inova 500 MHz (MestReNova Software: version 14.1.2-25024) or a Bruker 400 MHz using common deuterated solvens such as deuterium oxide, chloroform-d, or methanol-d₄. LCMS was accomplished using a Shimadzu LC-20AD Pump and LCMS-2020 Single Quadrupole Liquid Chromatograph Mass Spectrometer. Mass spectrometry was done using an Agilent 1290 UHPLC system with G4212 DAD and a G6140 MS detector (ESI: positive or negative ion mode). The samples were analyzed using an Agilent Eclipse-Plus C₁₈ column (2.1 mm×50 mm) with gradient elution (Mobile Phase A: 0.05% TFA in water; Mobile Phase B: acetonitrile with 0.1% AcOH or 0.025% TFA) at a flow rate of 0.5-1.0 mL/min. Gas chromatography analyses were conducted using an Agilent GC equipped with a flame ionization detector using a Agilent J&W CYCLOSIL-B (30 μm×250 μm×0.25 μm) column (Conditions: Inlet Temperature: 200° C.; Run Time: 60 min; Flow: 1 mL/min; Carrier Gas: Helium; Detector Temperature: 240° C.).

### Preparation of (2S,3R,5R,6S)-5-(benzoyloxy)-2-methyl-6-[(trichloroethanimidoyl) oxy]oxan-3-yl benzoate (C-01)

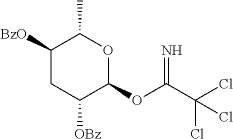

(2S,3R,5R)-5-(Benzoyloxy)-6-hydroxy-2-methyloxan-3-yl benzoate (40.0 g, 112 mmol, 1.00 eq.) was dissolved in DCM (200 mL) and trichloroacetonitrile (81.0 g, 561 mmol, 56.2 mL, 5.00 eq.) was added to the reaction. The mixture was degassed with nitrogen 3 times and cooled to 0° C. DBU (8.54 g, 56.1 mmol, 8.46 mL, 0.50 eq.) was added dropwise to the reaction at 0° C. and stirred at 0° C. for 1.5 h. TLC showed the reaction was complete (3:1 petroleum ether-ethyl acetate, SM R_{f}=0.45, R_{f} (C-01)=0.80). The resulting black liquid C-01 was used directly without further purification.

### Preparation of (2S,3R,5R,6S)-5-((tert-butyldiphenylsilyl)oxy)-6-methyl-2-(2,2,2-trichloro-1-iminoethoxy)tetrahydro-2H-pyran-3-yl benzoate (C-02)

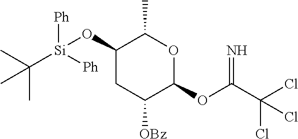

To a stir solution of (2R,3R,5R,6S)-5-((tert-butyldiphenylsilyl)oxy)-2-hydroxy-6-methyltetrahydro-2H-pyran-3-yl benzoate (7.30 g, 14.90 mmol) in DCM (50 mL) was added CCl₃CN (3.10 mL, 29.80 mmol) and DBU (0.210 mL, 1.50 mmol). The resulting mixture was stirred at room temperature under nitrogen for 18 h. The reaction was concentrated in vacuo then purified by the column chromatography (220 g silica gel, 0% to 30% acetone in hexanes) to afford C-02 (4.5 g, 48% yield) as a white foam. ¹H NMR (500 MHz, CDCl₃) δ 8.65 (s, 1H), 7.81-7.76 (m, 2H), 7.71-7.65 (m, 4H), 7.61 (ddt, J=8.8, 7.4, 1.3 Hz, 1H), 7.46-7.41 (m, 2H), 7.40-7.37 (m, 1H), 7.37-7.32 (m, 3H), 7.32-7.29 (m, 2H), 6.14 (s, 1H), 5.16 (dt, J=4.5, 2.1 Hz, 1H), 4.09-4.01 (m, 1H), 3.76 (ddd, J=11.1, 9.3, 4.4 Hz, 1H), 2.12 (ddd, J=14.1, 11.2, 3.0 Hz, 1H), 2.07-2.00 (m, 1H), 1.35 (d, J=6.2 Hz, 3H), 1.08 (s, 9H). Mass Analysis (ESI, +ve)=634.2 [M+H].

Using the above protocols, Table 1 summarizes additional carbohydrate starting materials that are prepared from commercial reagents after installing suitable protecting groups.

TABLE 1
Trichloroethanimidoyl Carbohydrates.

| # | Carbohydrate Core | Structure |
|---|---|---|
| C-01 | L-Rhamnose (6-Deoxy-L-mannose) CAS# 10030-85-0 | 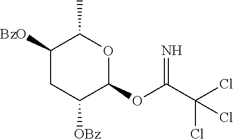 |
| C-02 | L-Rhamnose (6-Deoxy-L-mannose) CAS# 10030-85-0 | 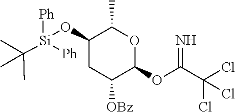 |
| C-03 | L-Rhamnose (6-Deoxy-L-mannose) CAS# 10030-85-0 | 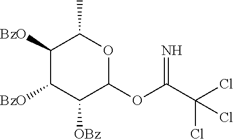 |
| C-04 | L-(−)-Fucose (6-Deoxy-L-galactose) CAS # 2438-80-4 | 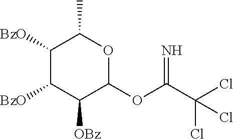 |
| C-05 | L-(−)-Mannose CAS # 10030-80-5 | 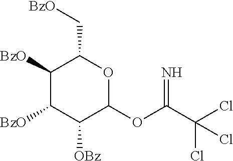 |
| C-06 | D-Glucose CAS# 492-62-6 | 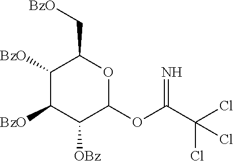 |

### Preparation of (2R)-hex-5-en-2-ol (I-01)

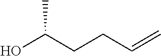

(R)-(+)-Propylene oxide (99.0 g, 1.70 mol, 119 mL, 1.00 eq.) was dissolved in THF (534 mL). Copper (I) bromide (24.4 g, 170 mmol, 5.19 mL, 0.10 eq.) was added to the reaction mixture as a solid. The reaction was cooled to −70° C. under nitrogen atmosphere and allylmagnesium bromide (1 M, 2.22 L, 1.30 eq.) was added to the reaction dropwise over 4 h. The reaction was warmed to 15° C. and stirred at 15° C. for 4 h. TLC showed the reaction was complete (5:1 petroleum ether-ethyl acetate, R_{f} (1-01)=0.39). The reaction was quenched with aqueous ammonium chloride (1.0 L) at 0° C. and then extracted with MTBE (3×5.0 L). The layers were separated and the organic layers were washed with brine (2×2.0 L). The combined organic layers were dried over sodium sulfate, filtered, and concentrate in vacuo at 30° C. The residue was purified by column chromatography (silica gel, 5:1 petroleum ether-ethyl acetate) to afford I-01 (43.3 g, 25% yield) as a yellow oil. ¹H NMR (400 MHz, CDCl₃) δ 5.87-5.80 (m, 1H), 5.06-4.95 (m, 1H), 3.84-3.82 (m, 1H), 3.81-3.79 (m, 1H), 2.18-2.11 (m, 1H), 1.61-1.52 (m, 2H), 1.19 (d, J=6.0 Hz, 3H). Chiral GC: 99.9% ee.

### Preparation of (2S,3R,5R,6R)-5-(benzoyloxy)-6-[(2R)-hex-5-en-2-yloxy]-2-methyloxan-3-yl benzoate (T-01)

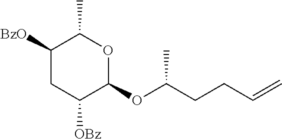

To the crude solution of C-01 was added 4 Å molecular sieves and the reaction was degassed with nitrogen 3 times. Compound I-01 (12.3 g, 123 mmol, 1.10 eq.) was added to the reaction at 25° C. and the mixture was cooled to −20° C. TMSOTf (12.4 g, 55.9 mmol, 10.1 mL, 0.50 eq.) was added dropwise to the reaction at −20° C. The mixture was stirred at −20° C. for 1.5 h. TLC showed the reaction was complete (3:1 petroleum ether-ethyl acetate, SM R_{f} (C-01)=0.80, R_{f} (T-01)=0.88). Water (500 mL) was added to quench the reaction and the mixture was extracted with DCM (3×300 mL). The layers were separated and the organic layers were washed with brine (2×50 mL). The combined organic layers were dried over sodium sulfate, filtered, and concentrate in vacuo. The residue was purified by column chromatography (silica gel, 2:1 petroleum ether-ethyl acetate) to afford T-01 (29.0 g, 59% yield) as a yellow oil. ¹H NMR (400 MHz, CDCl₃) δ 8.14-8.05 (m, 4H), 7.59-7.27 (m, 6H), 5.94-5.86 (m, 1H), 5.17-4.98 (m, 5H), 4.17-4.13 (m, 1H), 3.92-3.87 (m, 1H), 2.46-2.42 (m, 1H), 2.27-2.20 (m, 3H), 1.79-1.63 (m, 2H), 1.32 (d, J=4.2 Hz, 3H), 1.23 (d, J=4.2 Hz, 3H).

### Preparation of (2R,3R,5R,6S)-2-[(2R)-hex-5-en-2-yloxy]-6-methyloxane-3,5-diol (T-02)

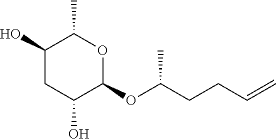 [SMILES: C=CCC[C@@H](C)O[C@@H]1O[C@@H](C)[C@H](O)C[C@H]1O]

Compound T-01 (23.0 g, 52.4 mmol, 1.00 eq.) was dissolved into THF (160 mL) and then water (230 mL) was added at 25° C. LiOH.H₂O (22.0 g, 524 mmol, 10.0 eq.) was added as a solid to the reaction mixture and the reaction was heated to 70° C. After stirring for 16 h, TLC showed the reaction was complete (5:1 petroleum ether-ethyl acetate, SM R_{f} (T-01)=0.24, R_{f}(T-02)=0.00). The mixture was concentrated at 40° C. in vacuo to remove THF. Water (400 mL) was added and the mixture was extracted with ethyl acetate (3×400 mL). The layers were separated and the organic layers were washed with brine (2×400 mL). The combined organic layers were dried over sodium sulfate, filtered, and concentrate in vacuo to afford T-02 (12 g, crude) as a colorless oil, which was used directly without further purification.

### Preparation of tert-butyl({[(2S,3R,5R,6R)-5-[(tert-butyldimethylsilyl)oxy]-6-[(2R)-hex-5-en-2-yloxy]-2-methyloxan-3-yl]oxy})dimethylsilane (T-03)

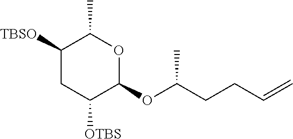 [SMILES: C=CCC[C@@H](C)O[C@@H]1O[C@@H](C)[C@H](O[Si](C)(C)C(C)(C)C)C[C@H]1O[Si](C)(C)C(C)(C)C]

Compound T-02 (11.0 g, 47.7 mmol, 1.00 eq.) was dissolved in THF (70 mL) and silver nitrate (20.2 g, 119 mmol, 2.50 eq.) and imidazole (13.0 g, 191 mmol, 4.00 eq.) were added as solids. The reaction was cooled to 0° C. under nitrogen atmosphere tert-Butyldimethylsilyl chloride (21.6 g, 143 mmol, 3.00 eq.) was added to the reaction and stir for 16 h at 25° C. TLC showed the reaction was complete. Water (200 mL) was added and the mixture was extracted with ethyl acetate (3×150 mL). The layers were separated and the organic layers were washed with brine (2×100 mL). The combined organic layers were dried over sodium sulfate, filtered, and concentrate in vacuo. The residue was purified by column chromatography (silica gel, 20:1 to 10:1 petroleum ether-ethyl acetate) to afford T-03 (14.0 g, 64% yield) as a yellow oil.

### Preparation of (3R,5R,6R)-2-methyl-6-(pent-4-en-1-yloxy)tetrahydro-2H-pyran-3,5-diyl dibenzoate (T-04)

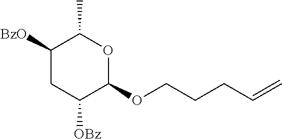

To a solution of (2R,3R,5R)-2-hydroxy-6-methyltetrahydro-2H-pyran-3,5-diyl dibenzoate (0.300 g, 0.842 mmol) in DCM (15 mL) was added 4-penten-1-ol (0.260 mL, 2.52 mmol) and 4 Å molecular sieve (0.100 g). The reaction mixture was cooled to 0° C. followed by adding BF₃.Et₂O (0.420 mL, 3.37 mmol) dropwise via the syringe. The resulting mixture was warmed up to room temperature and stirred for 4 h then quenched with triethylamine (2 mL). The solvent was concentrated in vacuo and the crude product was purified by the column chromatography (24 g silica gel, 0% to 30% acetone in hexanes) to afford T-04 (0.200 g, 56%) as a colorless oil. ¹H NMR (500 MHz, CDCl₃) δ 8.16-8.11 (m, 2H), 8.08-8.03 (m, 2H), 7.64-7.57 (m, 2H), 7.52-7.45 (m, 4H), 5.88 (ddt, J=16.9, 10.2, 6.6 Hz, 1H), 5.26-5.17 (m, 2H), 5.14-5.00 (m, 2H), 4.85 (s, 1H), 4.10 (dd, J=9.7, 6.2 Hz, 1H), 3.81 (dt, J=9.7, 6.6 Hz, 1H), 3.55 (dt, J=9.6, 6.4 Hz, 1H), 2.44 (dt, J=13.4, 3.9 Hz, 1H), 2.28-2.20 (m, 3H), 1.84-1.75 (m, 2H), 1.32 (d, J=6.2 Hz, 3H). Mass Analysis (ESI, +ve)=425.2 [M+H].

### Preparation of (2R,3R,5R,6S)-5-((tert-butyldiphenylsilyl)oxy)-2-(((R)-hex-5-en-2-yl)oxy)-6-methyltetrahydro-2H-pyran-3-yl benzoate (T-05)

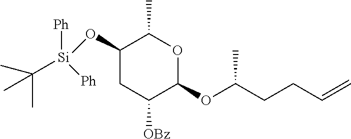

To a stirred solution of C-02 (0.570 g, 0.900 mmol) in DCM (5 mL) was added (2R)-(−)-hex-5-en-2-ol (0.165 mL, 1.35 mmol). The resulting mixture was cooled to 0° C. followed by treatment with TMSOTf (0.080 mL, 0.450 mmol). The reaction mixture was warmed to room temperature and stirred under nitrogen for 2 h. The resulting mixture was partitioned between water (5 mL) and DCM (10 mL). The organic layer was washed with saturated aqueous NaHCO₃ (5 mL), brine (5 mL), dried over magnesium sulfate, filtered, and concentrated. The crude product was purified by the column chromatography (40 g silica gel, 0% to 30% acetone in hexanes) to afford T-05 (0.420 g, 82% yield) as a light brown oil. ¹H NMR (500 MHz, CDCl₃) δ 7.77-7.73 (m, 2H), 7.72-7.66 (m, 4H), 7.58 (ddt, J=8.8, 7.2, 1.3 Hz, 1H), 7.43-7.38 (m, 3H), 7.37-7.29 (m, 5H), 5.91 (ddt, J=16.9, 10.2, 6.5 Hz, 1H), 5.12 (dq, J=17.1, 1.7 Hz, 1H), 5.07-5.02 (m, 1H), 4.90 (td, J=3.1, 1.4 Hz, 1H), 4.78 (s, 1H), 3.96-3.89 (m, 1H), 3.87-3.79 (m, 1H), 3.68 (ddd, J=11.2, 9.2, 4.3 Hz, 1H), 2.31-2.16 (m, 2H), 2.06 (ddd, J=14.0, 11.3, 3.0 Hz, 1H), 1.93-1.86 (m, 1H), 1.76 (dddd, J=13.4, 9.3, 7.4, 5.9 Hz, 1H), 1.65-1.58 (m, 1H), 1.29 (d, J=6.2 Hz, 3H), 1.17 (d, J=6.1 Hz, 3H), 1.08 (s, 9H). Mass Analysis (ESI, +ve)=573.2 [M+H].

Using the above protocols, the following carbohydrate alkane starting materials are made from the compounds in Table 2 and terminal alkene alcohols. Disubstituted alkene alcohols are made from ketones (e.g. acetone, cyclopropanone, cyclobutanone) by reaction with 3-butenylmagnesium bromide.

TABLE 2
Carbohydrates Terminal Alkenes.

| # | Structure |
|---|---|
| T-01 | 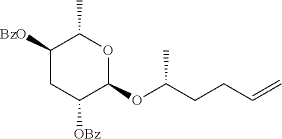 |
| T-02 | 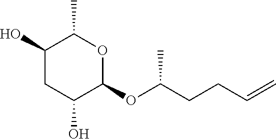 [SMILES: C=CCC[C@@H](C)O[C@@H]1O[C@@H](C)[C@H](O)C[C@H]1O] |
| T-03 | 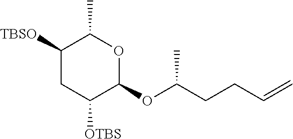 [SMILES: C=CCC[C@@H](C)O[C@@H]1O[C@@H](C)[C@H](O[Si](C)(C)C(C)(C)C)C[C@H]1O[Si](C)(C)C(C)(C)C] |
| T-04 | 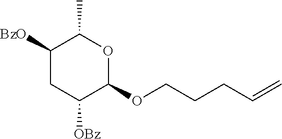 |
| T-05 | 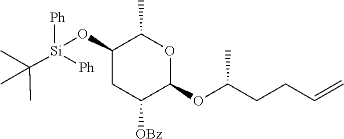 |
| T-06 | 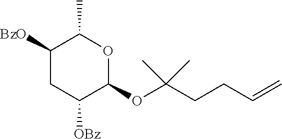 |
| T-07 | 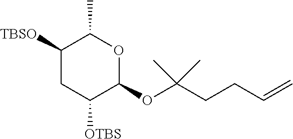 [SMILES: C=CCCC(C)(C)O[C@@H]1O[C@@H](C)[C@H](O[Si](C)(C)C(C)(C)C)C[C@H]1O[Si](C)(C)C(C)(C)C] |
| T-08 | 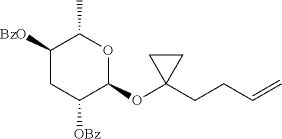 |
| T-09 | 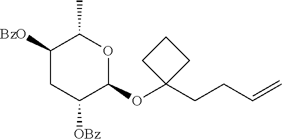 |
| T-10 | 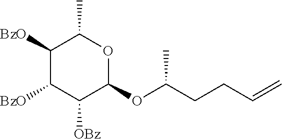 |
| T-11 | 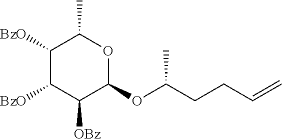 |
| T-12 | 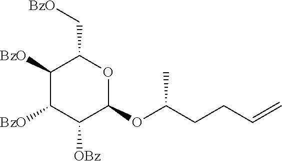 |
| T-13 | 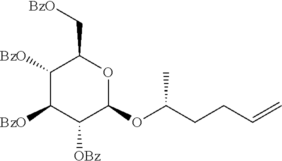 |
| T-14 | 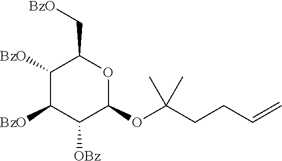 |

### Preparation of tert-butyl({[(2S,3R,5R,6R)-5-[(tert-butyldimethylsilyl)oxy]-2-methyl-6-{[(2R)-4-(oxiran-2-yl)butan-2-yl]oxy}oxan-3-yl]oxy})dimethylsilane (I-02)

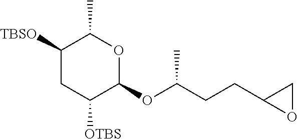 [SMILES: C[C@H](CCC1CO1)O[C@@H]1O[C@@H](C)[C@H](O[Si](C)(C)C(C)(C)C)C[C@H]1O[Si](C)(C)C(C)(C)C]

Compound T-03 (6.00 g, 13.0 mmol, 1.00 eq.) was dissolved into DCM (42 mL). m-CPBA (3.95 g, 18.3 mmol, 80% purity, 1.40 eq.) was added as a solid to the reaction mixture at 0° C. The reaction mixture was stirred at 25° C. for 16 h. TLC showed the reaction was complete (5:1 petroleum ether-ethyl acetate, SM R_{f}(T-03)=0.61, R_{f} (I-02)=0.43). The mixture was filtered, the solid was washed with DCM, and the filtrate was collected. The organic filtrate was washed with aqueous NaHSO₃ (3×70 mL) and aqueous NaHCO₃ (3×70 mL). The combined organic layers were dried over sodium sulfate, filtered, and concentrate in vacuo. The residue was purified by column chromatography (silica gel, 10:1 petroleum ether-ethyl acetate) to afford I-02 (5.78 g, 81% yield) as a colorless oil. ¹H NMR (400 MHz, CDCl₃) δ 4.53 (s, 1H), 3.89-3.49 (m, 4H), 2.93 (brs, 1H), 2.79-2.75 (m, 1H), 2.50-2.43 (m, 1H), 1.80-1.41 (m, 6H), 1.19-1.11 (m, 6H), 0.90-0.87 (m, 18H), 0.00 (s, 12H).

### Preparation of (4R)-4-{[(2R,3R,5R,6S)-3,5-bis[(tert-butyldimethylsilyl)oxy]-6-methyloxan-2-yl]oxy}pentanal (I-03)

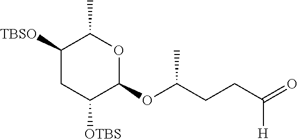 [SMILES: C[C@H](CCC=O)O[C@@H]1O[C@@H](C)[C@H](O[Si](C)(C)C(C)(C)C)C[C@H]1O[Si](C)(C)C(C)(C)C]

Compound T-03 (3.00 g, 6.54 mmol, 1.00 eq.) was dissolved in DCM (100 mL). Ozone was bubbled into the mixture at −70° C. for 10 min until the solution turns blue. Oxygen was bubbled into the reaction mixture at −70° C. for 20 min until the blue solution turns colorless. Triphenylphosphine (5.14 g, 19.6 mmol, 3.00 eq.) was added to the reaction mixture as a solid. The reaction was stirred at 25° C. for 16 h. TLC showed the reaction was complete (5:1 petroleum ether-ethyl acetate). The solvent was concentrate in vacuo and the residue was purified by column chromatography (silica gel, 5:1 petroleum ether-ethyl acetate) to afford I-03 (2.02 g, 67% yield) as a colorless oil. ¹H NMR (400 MHz, CDCl₃) δ 9.81 (s, 1H), 4.54 (s, 1H), 3.88-3.50 (m, 4H), 2.60-2.50 (m, 2H), 1.86-1.76 (m, 4H), 1.18-1.13 (m, 6H), 0.90-0.74 (m, 18H), 0.06 (s, 12H).

### Preparation of (2R,3R,5R,6S)-5-((tert-butyldiphenylsilyl)oxy)-2-(((2R)-5,6-dihydroxyhexan-2-yl)oxy)-6-methyltetrahydro-2H-pyran-3-yl benzoate (I-04)

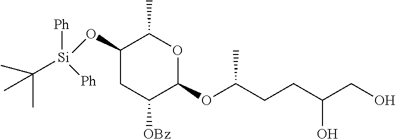

To a solution of T-05 (0.300 g, 0.524 mmol) in THF (3 mL) and water (1 mL) was added 4-methylmorpholine N-oxide (0.25 mL, 1.05 mmol) and osmium tetroxide (0.34 mL, 0.0524 mmol, 4% weight in water). The reaction mixture was stirred at room temperature under nitrogen for 18 h. The resulting mixture was quenched with saturated aqueous NaHCO₃ (2 mL) then partitioned between ethyl acetate (10 mL) and water (5 mL). The aqueous layer was extracted with ethyl acetate (2×10 mL). The combined organic layers were dried over magnesium sulfate, filtered, and concentrated. The crude product was purified by the column chromatography (24 g silica gel, 0% to 40% acetone in hexanes) to afford I-04 (0.280 g, 88% yield) as a colorless paste. ¹H NMR (500 MHz, CDCl₃) δ 7.76-7.73 (m, 2H), 7.72-7.65 (m, 4H), 7.58 (ddt, J=8.7, 7.4, 1.3 Hz, 1H), 7.43-7.38 (m, 3H), 7.38-7.29 (m, 6H), 4.90 (s, 1H), 4.79 (s, 1H), 3.93-3.85 (m, 2H), 3.84-3.78 (m, 1H), 3.75 (ddt, J=10.1, 6.8, 3.6 Hz, 1H), 3.71-3.64 (m, 1H), 3.58-3.51 (m, 1H), 2.35 (dd, J=13.0, 4.3 Hz, 1H), 2.10-2.00 (m, 2H), 1.95-1.87 (m, 2H), 1.83-1.69 (m, 2H), 1.69-1.61 (m, 2H), 1.31-1.27 (m, 3H), 1.19 (d, J=6.1 Hz, 3H), 1.08 (s, 9H). Mass Analysis (ESI, +ve)=607.2 [M+H].

### Preparation of (2R,3R,5R,6S)-5-((tert-butyldiphenylsilyl)oxy)-6-methyl-2-(((R)-5-oxopentan-2-yl)oxy)tetrahydro-2H-pyran-3-yl benzoate (I-05)

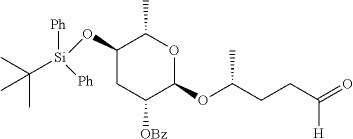

To a solution of I-04 (0.280 g, 0.462 mmol) in THF (2 mL) and water (2 mL) was added NaIO₄ (0.224 g, 1.05 mmol). The reaction mixture was stirred at room temperature under nitrogen for 18 h. The resulting mixture was quenched with saturated aqueous NaHCO₃ (2 mL) then partitioned between ethyl acetate (10 mL) and water (5 mL). The aqueous layer was extracted with ethyl acetate (2×10 mL). The combined organic layers were dried over magnesium sulfate, filtered, and concentrated. The crude product was purified by the column chromatography (24 g silica gel, 0% to 30% acetone in hexanes) to afford I-05 (0.150 g, 57% yield) as a colorless paste. ¹H NMR (500 MHz, CDCl₃) δ 9.89 (t, J=1.6 Hz, 1H), 7.76-7.72 (m, 2H), 7.72-7.66 (m, 4H), 7.60-7.56 (m, 1H), 7.43-7.38 (m, 3H), 7.38-7.29 (m, 5H), 4.88 (td, J=3.2, 2.0 Hz, 1H), 4.77 (s, 1H), 3.91-3.80 (m, 2H), 3.67 (ddd, J=11.2, 9.1, 4.3 Hz, 1H), 2.64 (dddd, J=10.9, 8.3, 6.8, 1.6 Hz, 2H), 2.01 (ddd, J=14.0, 11.2, 3.0 Hz, 1H), 1.96-1.86 (m, 3H), 1.29 (d, J=6.2 Hz, 3H), 1.19 (d, J=6.1 Hz, 3H), 1.08 (s, 8H). Mass Analysis (ESI, +ve)=575.1 [M+H].

### Preparation of tert-butyl (2E,6R)-6-{[(2R,3R,5R,6S)-3,5-dihydroxy-6-methyloxan-2-yl]oxy}hept-2-enoate (1)

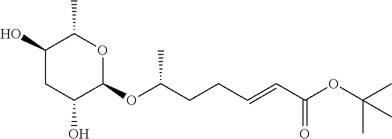 [SMILES: C[C@H](CC/C=C/C(=O)OC(C)(C)C)O[C@@H]1O[C@@H](C)[C@H](O)C[C@H]1O]

Compound T-02 (5.00 g, 21.7 mmol, 1.00 eq.) was dissolved into DCM (330 mL) and tert-butyl acrylate (13.9 g, 108 mmol, 15.7 mL, 5.00 eq.) was added to the reaction mixture at 15° C. Grubb's M204 catalyst (1.84 g, 2.17 mmol, 0.10 eq.) was added to the reaction at 15° C. and the mixture was degasses with nitrogen 3 times. The reaction was heated to 50° C. and stirred at 50° C. for 16 h. TLC showed the reaction was complete (5:1 petroleum ether-ethyl acetate, SM R_{f} (T-02)=0.50, R_{f} (1)=0.00). The solvent was concentrate in vacuo and the residue was purified by column chromatography (silica gel, 100% ethyl acetate) and then purified again by prep-HPLC (Column: Agela DuraShell C₁₈ 250×25 mm, 10 m; Mobile Phase A: Water 0.05% ammonia hydroxide (v/v), Mobile Phase B: ACN; Gradient B %: 12%-46%, Runtime: 22 min) to afford 1 (2.00 g, 6.05 mmol, 28% yield) as a reddish brown oil. ¹H NMR (400 MHz, CDCl₃) δ 6.94-6.87 (m, 1H), 5.79-5.75 (m, 1H), 4.79 (s, 1H), 3.85-3.81 (m, 2H), 3.67-3.62 (m, 2H), 2.32-2.29 (m, 2H), 1.48 (s, 9H), 1.28 (d, J=6.0 Hz, 3H), 1.15 (d, J=6.0 Hz, 3H). LCMS Analysis (positive mode)=353.2 [M+Na].

### Preparation of tert-butyl 2-[(3R)-3-{[(2R,3R,5R,6S)-3,5-bis[(tert-butyldimethylsilyl)oxy]-6-methyloxan-2-yl]oxy}butyl]cyclopropane-1-carboxylate (I-06)

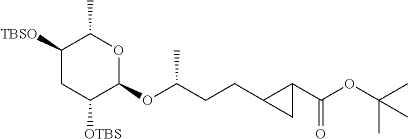 [SMILES: C[C@H](CCC1CC1C(=O)OC(C)(C)C)O[C@@H]1O[C@@H](C)[C@H](O[Si](C)(C)C(C)(C)C)C[C@H]1O[Si](C)(C)C(C)(C)C]

ert-Butyl 2-diethoxyphosphorylacetate (398 mg, 1.58 mmol, 1.50 eq.) was dissolved into 1,4-dioxane (3.5 mL) and cooled to 15° C. Sodium tert-butoxide (121 mg, 1.26 mmol, 1.20 eq.) was added to the reaction mixture under nitrogen atmosphere. The reaction mixture was stirred at 15° C. for 1 h. Compound I-03 (500 mg, 1.05 mmol, 1.00 eq.) was added and the mixture was heated at 130° C. for 16 h. TLC showed the reaction was complete (5:1 petroleum ether-ethyl acetate). Acetic acid was added to the mixture to quench the reaction and to bring the pH to 7. Brine (2.0 mL) and water (5.0 mL) were added to the mixture and the reaction was extracted with ethyl acetate (3×3.0 mL). The layers were separated and the organic layers were washed with aqueous NaHCO₃ (2×10 mL). The combined organic layers were dried over sodium sulfate, filtered, and concentrate in vacuo. The residue was purified by preparative TLC (silica gel, 5:1 petroleum ether-ethyl acetate) to afford I-06 (100 mg, 17% yield) as a colorless oil. ¹H NMR (400 MHz, CDCl₃) δ 4.54 (s, 1H), 3.86-3.74 (m, 2H), 3.66-3.56 (m, 2H), 1.80-1.78 (5H), 1.28 (s, 9H), 1.30-1.26 (m, 2H), 1.19-1.09 (m, 6H), 0.90-0.89 (m, 18H), 0.68-0.05 (m, 12H).

### Preparation of tert-butyl 2-[(3R)-3-{[(2R,3R,5R,6S)-3,5-dihydroxy-6-methyloxan-2-yl]oxy}butyl]cyclopropane-1-carboxylate (2)

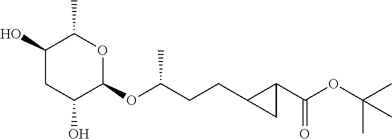 [SMILES: C[C@H](CCC1CC1C(=O)OC(C)(C)C)O[C@@H]1O[C@@H](C)[C@H](O)C[C@H]1O]

Compound I-06 (500 mg, 872 μmol, 1.00 eq.) was dissolved in THF (3.5 mL) and Et₃N.3HF (1.41 g, 8.73 mmol, 1.42 mL, 10.0 eq.) was added to the reaction mixture at 15° C. The reaction was stirred at 50° C. for 16 h. LCMS showed the reaction was complete. Aqueous NaHCO₃ was added to bring the pH to 8 and the mixture was concentrated in vacuo. The residue was purified by column chromatography (silica gel, 5:1 petroleum ether-ethyl acetate) to afford 2 (130 mg, 43% yield) as a colorless oil. ¹H NMR (400 MHz, CDCl₃) δ 4.70 (s, 1H), 3.84-3.80 (m, 2H), 3.66-3.58 (m, 2H), 2.10-2.01 (m, 1H), 1.87-1.76 (m, 2H), 1.71-1.51 (m, 4H), 1.44 (s, 9H), 1.35-1.19 (m, 6H), 1.15-1.03 (m, 4H), 0.66-0.56 (m, 1H). LCMS Analysis (positive mode)=367.2 [M+Na].

### Preparation of methyl (2E,6R)-6-{[(2R,3R,5R,6S)-3,5-bis[(tert-butyldimethylsilyl)oxy]-6-methyloxan-2-yl]oxy}hept-2-enoate (I-07)

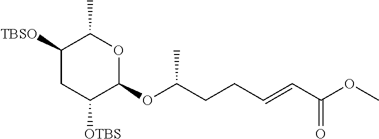 [SMILES: COC(=O)/C=C/CC[C@@H](C)O[C@@H]1O[C@@H](C)[C@H](O[Si](C)(C)C(C)(C)C)C[C@H]1O[Si](C)(C)C(C)(C)C]

Compound T-03 (10.0 g, 21.8 mmol, 1.00 eq.) was dissolved into DCM (300 mL) and methyl acrylate (9.38 g, 108 mmol, 9.81 mL, 5.00 eq.) was added to the reaction mixture at 25° C. Grubb's M204 catalyst (1.85 g, 2.18 mmol, 0.10 eq.) was added to the reaction mixture at 25° C. and then the mixture was heated to 50° C. The reaction mixture was stirred at 50° C. for 16 h. TLC showed the reaction was complete (5:1 petroleum ether-ethyl acetate, SM R_{f}(T-03)=0.61, R_{f} (I-07)=0.44). The solvent was concentrate in vacuo and the residue was purified by column chromatography (silica gel, 5:1 petroleum ether-ethyl acetate) to afford I-07 (8.3 g, 74% yield) as a colorless oil. ¹H NMR (400 MHz, CDCl₃) δ 6.97-6.80 (m, 1H), 5.79 (d, J=15.6 Hz, 1H), 4.48 (s, 1H), 4.07-4.03 (m, 2H), 3.71 (s, 3H), 3.59-3.45 (m, 2H), 2.31-2.22 (m, 2H), 1.74-1.69 (m, 4H), 1.19 (d, J=7.2 Hz, 3H), 1.12 (d, J=6.0 Hz, 3H), 0.84-0.83 (m, 18H), 0.01-0.00 (m, 12H). LCMS Analysis (positive mode)=539.3 [M+Na].

### Preparation of (2E,6R)-6-{[(2R,3R,5R,6S)-3,5-bis[(tert-butyldimethylsilyl)oxy]-6-methyloxan-2-yl]oxy}hept-2-enoic acid (I-08)

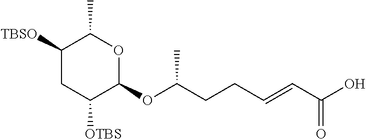 [SMILES: C[C@H](CC/C=C/C(=O)O)O[C@@H]1O[C@@H](C)[C@H](O[Si](C)(C)C(C)(C)C)C[C@H]1O[Si](C)(C)C(C)(C)C]

Compound I-07 (8.30 g, 16.0 mmol, 1.00 eq.) was dissolved in a mixture of methanol (83 mL), water (41.5 mL), and THF (58 mL). A solution of LiOH.H₂O (1.35 g, 32.1 mmol, 2.00 eq.) in water (41.5 mL) was added to the mixture at 25° C. The reaction was stirred at 50° C. for 3 h. TLC showed the reaction was complete (5:1 petroleum ether-ethyl acetate, SM R_{f} (I-07)=0.8, R_{f} (I-08)=0.17). 1N HCl was added to the reaction mixture to make the pH=2. The mixture was extracted with ethyl acetate (3×30 mL). The layers were separated and the organic layers were washed with brine (40 mL). The combined organic layers were dried over sodium sulfate, filtered, and concentrate in vacuo to afford I-08 (7.3 g, crude) as a brown oil, which was used directly without further purification.

### Compound cyclohexylmethyl (2E,6R)-6-{[(2R,3R,5R,6S)-3,5-bis[(tert-butyldimethylsilyl)oxy]-6-methyloxan-2-yl]oxy}hept-2-enoate (I-09)

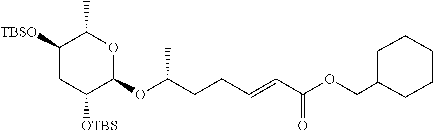 [SMILES: C[C@H](CC/C=C/C(=O)OCC1CCCCC1)O[C@@H]1O[C@@H](C)[C@H](O[Si](C)(C)C(C)(C)C)C[C@H]1O[Si](C)(C)C(C)(C)C]

Compound I-08 (1.00 eq.) and alcohol (3.00 eq.) were dissolved in DCM (3.5 mL). DCC (1.10 eq.) and DMAP (0.80 eq.) were added to the solution as solids and the reaction mixture was stirred at 25° C. for 16 h. TLC showed the reaction was complete (5:1 petroleum ether-ethyl acetate). The solvent was concentrate in vacuo and the residue was purified by column chromatography (silica gel, petroleum ether-ethyl acetate) to afford I-09 as a yellow oil which was used directly in the final deprotection step. ¹H NMR (400 MHz, CDCl₃) δ 6.97-6.91 (m, 1H), 5.82 (d, J=15.6 Hz, 1H), 4.48 (s, 1H), 3.88-3.47 (m, 6H), 2.29-2.11 (m, 2H), 1.74-1.65 (m, 9H), 1.25-0.80 (m, 14H), 0.84-0.83 (m, 18H), 0.00 (s, 12H).

The following compounds were prepared using the above procedure:

Compound benzyl (2E,6R)-6-{[(2R,3R,5R,6S)-3,5-bis[(tert-butyldimethylsilyl)oxy]-6-methyloxan-2-yl]oxy}hept-2-enoate (I-10):

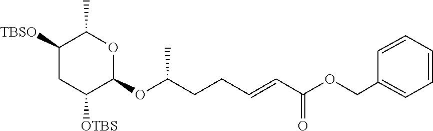 [SMILES: C[C@H](CC/C=C/C(=O)OCc1ccccc1)O[C@@H]1O[C@@H](C)[C@H](O[Si](C)(C)C(C)(C)C)C[C@H]1O[Si](C)(C)C(C)(C)C]

Yellow oil. ¹H NMR (400 MHz, CDCl₃) δ 7.31-7.28 (m, 5H), 7.02-6.96 (m, 1H), 5.84 (d, J=15.6 Hz, 1H), 5.11 (s, 2H), 4.48-4.47 (m, 1H), 3.75-3.70 (m, 2H), 3.59-3.46 (m, 2H), 2.30-2.22 (m, 2H), 1.74-1.65 (m, 4H), 1.20-1.02 (m, 8H), 0.84-0.82 (m, 18H), 0.00 (s, 12H).

Compound pyridin-3-ylmethyl (2E,6R)-6-{[(2R,3R,5R,6S)-3,5-bis[(tert-butyldimethylsilyl)oxy]-6-methyloxan-2-yl]oxy}hept-2-enoate (I-11):

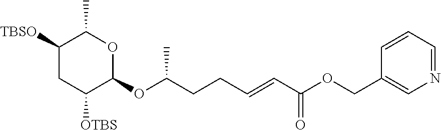 [SMILES: C[C@H](CC/C=C/C(=O)OCc1cccnc1)O[C@@H]1O[C@@H](C)[C@H](O[Si](C)(C)C(C)(C)C)C[C@H]1O[Si](C)(C)C(C)(C)C]

Yellow oil. ¹H NMR (400 MHz, CDCl₃) δ 8.58-8.51 (m, 2H), 7.66-7.64 (m, 1H), 7.26-7.22 (m, 1H), 7.01-6.95 (m, 1H), 5.83 (d, J=16.0 Hz, 1H), 5.11 (s, 2H), 4.48-4.47 (m, 1H), 3.75-3.70 (m, 2H), 3.59-3.48 (m, 2H), 2.31-2.23 (m, 1H), 1.74-1.68 (m, 4H), 1.20-1.02 (m, 9H), 0.84-0.68 (m, 18H), 0.00 (s, 12H).

Compound pyridin-4-ylmethyl (2E,6R)-6-{[(2R,3R,5R,6S)-3,5-bis[(tert-butyldimethylsilyl)oxy]-6-methyloxan-2-yl]oxy}hept-2-enoate (I-12):

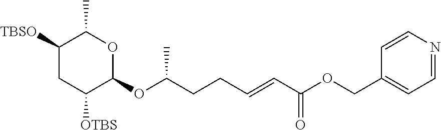 [SMILES: C[C@H](CC/C=C/C(=O)OCc1ccncc1)O[C@@H]1O[C@@H](C)[C@H](O[Si](C)(C)C(C)(C)C)C[C@H]1O[Si](C)(C)C(C)(C)C]

Yellow oil. ¹H NMR (400 MHz, CDCl₃) δ 8.55-8.53 (m, 2H), 7.15-7.10 (m, 2H), 7.03-7.69 (m, 1H), 5.88 (d, 15.6 Hz, 1H), 5.11 (s, 2H), 4.48-4.47 (m, 1H), 3.76-3.70 (m, 2H), 3.60-3.49 (m, 2H), 2.35-2.26 (m, 2H), 1.74-1.69 (m, 4H), 1.19-1.03 (m, 8H), 0.84-0.68 (m, 18H), 0.00 (s, 12H).

Compound 2-phenylethyl (2E,6R)-6-{[(2R,3R,5R,6S)-3,5-bis[(tert-butyldimethylsilyl)oxy]-6-methyloxan-2-yl]oxy}hept-2-enoate (I-13):

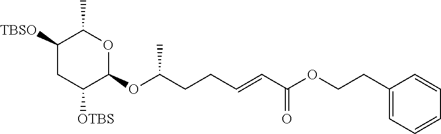 [SMILES: C[C@H](CC/C=C/C(=O)OCCc1ccccc1)O[C@@H]1O[C@@H](C)[C@H](O[Si](C)(C)C(C)(C)C)C[C@H]1O[Si](C)(C)C(C)(C)C]

Yellow oil. ¹H NMR (400 MHz, CDCl₃) δ 7.26-7.20 (m, 2H), 7.18-7.14 (m, 3H), 6.93-6.89 (m, 1H), 5.77 (d, J=15.6 Hz, 1H), 4.48-4.47 (m, 1H), 4.29-4.22 (m, 2H), 3.73-3.70 (m, 2H), 3.58-3.49 (m, 2H), 2.92-2.86 (m, 3H), 2.31-2.11 (m, 2H), 1.75-1.51 (m, 4H), 1.15-1.01 (m, 8H), 0.84-0.68 (m, 18H), 0.00 (s, 12H).

Compound cyclohexylmethyl (2E,6R)-6-{[(2R,3R,5R,6S)-3,5-dihydroxy-6-methyloxan-2-yl]oxy}hept-2-enoate (3):

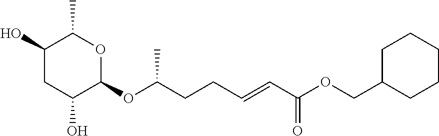 [SMILES: C[C@H](CC/C=C/C(=O)OCC1CCCCC1)O[C@@H]1O[C@@H](C)[C@H](O)C[C@H]1O]

Compound I-09 (1.00 eq.) was dissolved in THF (7 V) and Et₃N.3HF (10 eq.) was added. The reaction was stirred at 50° C. for 4 h. LCMS showed the reaction was complete. The solvent was concentrate in vacuo and the residue was purified by prep-HPLC (Column: Phenomena Luna C₁₈ 200 mm×40 mm, 10 μm; Mobile Phase A: Water w/0.2% formic acid and Mobile Phase B: ACN; Gradient B %: 30% to 70%; Runtime: 8 min) to obtain crude product. Purify the crude product by SFC Purify the crude product by SFC (Instrument: Thar SFC80 preparative SFC; Column: REGIS (S,S) WHELK-01 (250 mm×30 mm, 10 μm); Mobile Phase A: Supercritical CO₂ and Mobile Phase B: Ethanol; Isocratic: B %=30%; Flow rate: 55 mL/min; Wavelength: 220 nm; Column Temperature: 40° C. System Back Pressure: 100 bar; Runtime: 10 min) to afford 26.0 mg of 3 a colorless oil. ¹H NMR (400 MHz, CDCl₃) δ 7.00 (dt, J=15.6, 7.0 Hz, 1H), 5.85 (d, J=15.6 Hz, 1H), 4.70 (s, 1H), 3.94 (d, J=6.4 Hz, 2H), 3.87-3.77 (m, 4H), 3.67-3.52 (m, 2H), 2.42-2.27 (m, 2H), 2.17-2.02 (m, 1H), 1.82-1.57 (m, 1H), 1.28 (d, J=6.4 Hz, 3H), 1.26-1.17 (m, 2H), 1.16 (d, J=6.4 Hz, 3H), 1.07-0.92 (m, 2H). LCMS Analysis (positive mode)=393.2 [M+Na]; 98.9% LCMS purity.

The following compounds were prepared using the above procedure:

Compound benzyl (2E,6R)-6-{[(2R,3R,5R,6S)-3,5-dihydroxy-6-methyloxan-2-yl]oxy}hept-2-enoate (4):

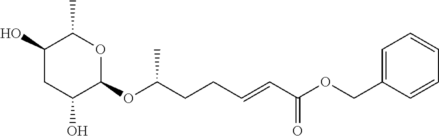 [SMILES: C[C@H](CC/C=C/C(=O)OCc1ccccc1)O[C@@H]1O[C@@H](C)[C@H](O)C[C@H]1O]

Compound 4: 6.5 mg of a colorless oil. ¹H NMR (400 MHz, CDCl₃) δ 7.38-7.32 (m, 5H), 7.05 (dt, J=15.6, 7.5 Hz, 1H), 5.90 (d, J=15.6 Hz, 1H), 5.18 (s, 2H), 4.69 (s, 1H), 3.86-3.80 (m, 2H), 3.63-3.54 (m, 2H), 2.35-2.31 (m, 2H), 2.06 (d, J=13.2 Hz, 1H), 1.81-1.56 (m, 5H), 1.26 (d, J=6.0 Hz, 3H), 1.15 (d, J=6.0 Hz, 3H). LCMS Analysis (positive mode)=387.2 [M+Na]; 92.7% LCMS purity.

Compound pyridin-3-ylmethyl (2E,6R)-6-{[(2R,3R,5R,6S)-3,5-dihydroxy-6-methyloxan-2-yl]oxy}hept-2-enoate (5):

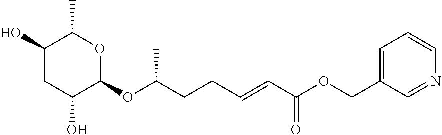 [SMILES: C[C@H](CC/C=C/C(=O)OCc1cccnc1)O[C@@H]1O[C@@H](C)[C@H](O)C[C@H]1O]

Compound 5: 26.1 mg of a colorless oil. ¹H NMR (400 MHz, CDCl₃) δ 8.64 (d, J=1.6 Hz, 1H), 8.59-8.57 (m, 1H), 7.71 (dd, J=6.0, 2.0 Hz, 1H), 7.30 (dd, J=7.6, 4.3 Hz, 1H), 7.07 (dm, J=15.6 Hz, 1H), 5.88 (dd, J=15.6, 1.6 Hz, 1H), 5.19 (s, 2H), 4.70 (s, 1H), 3.85-3.80 (m, 2H), 3.63-3.59 (m, 2H), 2.36-2.32 (m, 2H), 2.00-2.10 (m, 1H), 1.82-1.59 (m, 5H), 1.26 (d, J=6.0 Hz, 3H), 1.15 (d, J=6.0 Hz, 3H). LCMS Analysis (positive mode)=366.2 [M+H]; 98.3% LCMS purity.

Compound pyridin-4-ylmethyl (2E,6R)-6-{[(2R,3R,5R,6S)-3,5-dihydroxy-6-methyloxan-2-yl]oxy}hept-2-enoate (6):

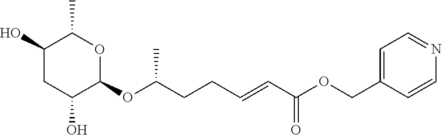 [SMILES: C[C@H](CC/C=C/C(=O)OCc1ccncc1)O[C@@H]1O[C@@H](C)[C@H](O)C[C@H]1O]

Compound 6: 26.4 mg of a colorless oil. ¹H NMR (400 MHz, CDCl₃) δ 8.604 (d, J=4.8 Hz, 2H), 7.268 (d, J=4.0 Hz, 2H), 7.11 (dt, J=15.6, 7.0 Hz, 1H), 5.943 (d, J=14.4 Hz, 1H), 5.192 (s, 2H), 4.709 (s, 1H), 3.89-3.81 (m, 2H), 3.66-3.59 (m, 2H), 2.47-2.32 (m, 2H), 2.12-2.04 (m, 1H), 1.87-1.64 (m, 5H), 1.27 (d, J=6.0 Hz, 3H), 1.16 (d, J=6.0 Hz, 3H). LCMS Analysis (positive mode)=366.2 [M+H]; 99.7% LCMS purity.

Compound 2-phenylethyl (2E,6R)-6-{[(2R,3R,5R,6S)-3,5-dihydroxy-6-methyloxan-2-yl]oxy}hept-2-enoate (7):

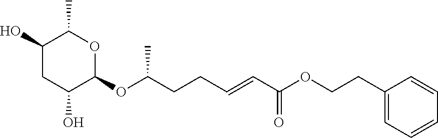 [SMILES: C[C@H](CC/C=C/C(=O)OCCc1ccccc1)O[C@@H]1O[C@@H](C)[C@H](O)C[C@H]1O]

Compound 7: 26.1 mg of a colorless oil. ¹H NMR (400 MHz, CDCl₃) δ 7.33-7.22 (m, 5H), 7.00 (dd, J=15.6, 7.8 Hz, 1H), 5.84 (d, J=15.6 Hz, 1H), 4.70 (s, 1H), 4.35 (t, J=7.2 Hz, 2H), 3.84-3.84 (m, 2H), 3.63-3.57 (m, 2H), 2.97 (t, J=7.2 Hz, 2H), 2.30-2.20 (m, 2H), 2.08 (d, J=12.8 Hz, 1H), 1.83-1.45 (m, 5H), 1.27 (d, J=6.0 Hz, 3H), 1.16 (d, J=6.0 Hz, 3H). LCMS Analysis (positive mode)=401.2 [M+Na]; 87.1% LCMS purity.

### Preparation of (2R,3R,5R,6S)-5-((tert-butyldiphenylsilyl)oxy)-2-(((R,E)-7-isopropoxy-7-oxohept-5-en-2-yl)oxy)-6-methyltetrahydro-2H-pyran-3-yl benzoate (I-14)

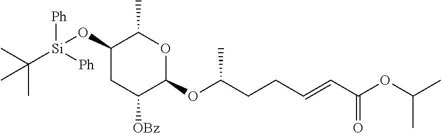

Using the same procedure to make I-07, to a solution of T-05 (0.200 g, 0.349 mmol) in DCM (5 mL) was added isopropyl acrylate (0.090 mL, 0.699 mmol) and Grubb's M204 catalyst (0.015 g, 0.0174 mmol) and after isolation and purification afforded I-14 (0.130 g, 57% yield) as a colorless paste. ¹H NMR (500 MHz, CDCl₃) δ 7.76-7.73 (m, 2H), 7.72-7.66 (m, 4H), 7.58 (ddt, J=8.7, 7.1, 1.3 Hz, 1H), 7.44-7.38 (m, 3H), 7.38-7.29 (m, 5H), 7.04 (dt, J=15.7, 6.8 Hz, 1H), 5.90 (dt, J=15.7, 1.6 Hz, 1H), 5.10 (p, J=6.3 Hz, 2H), 4.89 (dt, J=4.5, 2.1 Hz, 1H), 4.77 (s, 1H), 3.91-3.80 (m, 2H), 3.68 (ddd, J=11.2, 9.2, 4.3 Hz, 1H), 2.51-2.25 (m, 3H), 2.04 (ddd, J=14.0, 11.2, 3.0 Hz, 2H), 1.90 (dt, J=14.0, 3.8 Hz, 1H), 1.79 (dddd, J=13.4, 9.4, 5.7, 3.8 Hz, 1H), 1.72-1.63 (m, 1H), 1.29 (dd, J=6.3, 5.1 Hz, 9H), 1.17 (d, J=6.1 Hz, 3H), 1.08 (s, 9H). Mass Analysis (ESI, +ve)=660.2 [M+H].

### Preparation of isopropyl (R,E)-6-(((2R,3R,5R,6S)-5-((tert-butyldiphenylsilyl)oxy)-3-hydroxy-6-methyltetrahydro-2H-pyran-2-yl)oxy)hept-2-enoate (I-15)

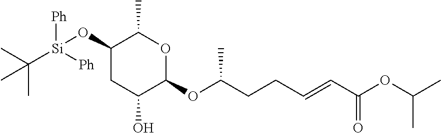 [SMILES: CC(C)OC(=O)/C=C/CC[C@@H](C)O[C@@H]1O[C@@H](C)[C@H](O[Si](c2ccccc2)(c2ccccc2)C(C)(C)C)C[C@H]1O]

To a solution of I-14 (0.130 g, 0.197 mmol) in isopropanol (5 mL) was added 1% aq. NaOH (2.3 mL, 0.591 mmol). The reaction mixture was stirred at room temperature under nitrogen for 18 h. The resulting mixture was partitioned between ethyl acetate (10 mL) and water (5 mL). The aqueous layer was extracted with ethyl acetate (2×10 mL). The combined organic layers were dried over magnesium sulfate, filtered, and concentrated. The crude product was purified by the column chromatography (24 g silica gel, 0% to 30% acetone in hexanes) to afford I-15 (0.060 g, 55% yield) as a colorless oil. ¹H NMR (500 MHz, CDCl₃) δ 7.75-7.66 (m, 4H), 7.48-7.43 (m, 2H), 7.43-7.37 (m, 4H), 7.02 (dt, J=15.6, 6.8 Hz, 1H), 5.89 (dt, J=15.6, 1.6 Hz, 1H), 5.10 (p, J=6.3 Hz, 1H), 4.63-4.58 (m, 1H), 3.82 (ddd, J=7.8, 6.3, 5.1 Hz, 1H), 3.77 (dd, J=8.9, 6.2 Hz, 1H), 2.45-2.27 (m, 3H), 1.90-1.71 (m, 4H), 1.69-1.61 (m, 2H), 1.29 (d, J=6.3 Hz, 6H), 1.19 (d, J=6.2 Hz, 3H), 1.14 (d, J=6.1 Hz, 3H), 1.08 (s, 9H). Mass Analysis (ESI, +ve)=555.1 [M+H].

### Preparation of isopropyl (R,E)-6-(((2R,3R,5R,6S)-3,5-dihydroxy-6-methyltetrahydro-2H-pyran-2-yl)oxy)hept-2-enoate (8)

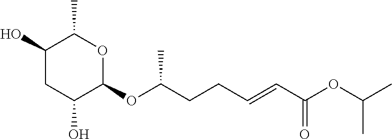 [SMILES: CC(C)OC(=O)/C=C/CC[C@@H](C)O[C@@H]1O[C@@H](C)[C@H](O)C[C@H]1O]

To a solution of I-15 (0.055 g, 0.099 mmol) in THF (3 mL) was added 1.0 M TBAF (0.500 mmol, 0.500 mL) in THF. The reaction mixture was stirred at room temperature under nitrogen for 18 h. The resulting mixture was concentrated and the crude product was purified by the column chromatography (24 g silica gel, 0% to 15% MeOH in DCM) to afford 8 (0.013 g, 42% yield) as a colorless oil. ¹H NMR (500 MHz, CDCl₃) δ 7.01 (dt, J=15.7, 6.8 Hz, 1H), 5.84 (dt, J=15.6, 1.6 Hz, 1H), 5.08 (p, J=6.2 Hz, 1H), 4.77-4.69 (m, 1H), 3.90-3.85 (m, 1H), 3.83 (q, J=2.9 Hz, 1H), 3.70-3.65 (m, 1H), 3.60 (ddd, J=11.0, 9.1, 4.4 Hz, 1H), 2.34 (ttd, J=8.5, 6.8, 1.7 Hz, 2H), 2.10 (dddd, J=13.0, 4.5, 3.5, 1.0 Hz, 1H), 1.85 (ddd, J=13.2, 10.9, 3.0 Hz, 2H), 1.78-1.69 (m, 3H), 1.30 (d, J=6.1 Hz, 3H), 1.28 (d, J=6.3 Hz, 6H), 1.18 (d, J=6.1 Hz, 3H). Mass Analysis (ESI, +ve)=317.2.1 [M+H].

### Preparation of (2R,3R,5R,6S)-2-(((2R)-7-(tert-butoxy)-5-hydroxy-7-oxoheptan-2-yl)oxy)-5-((tert-butyldiphenylsilyl)oxy)-6-methyltetrahydro-2H-pyran-3-yl benzoate (I-16)

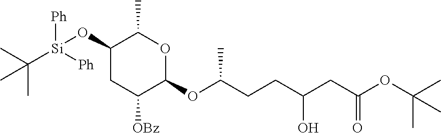

To an oven-dry 25-mL 3-neck round-bottom flask, equipped with a low-temperature thermometer and nitrogen inlet, was added tert-butyl acetate (0.140 mL, 1.044 mmol) and THF (5 mL). The resulting mixture was cooled to −75° C. followed by addition of 1.96 M LDA (0.400 mL, 0.783 mmol) dropwise via syringe. The reaction mixture was stirred at −75° C. for 2 h then (2R,3R,5R,6S)-5-((tert-butyldiphenylsilyl)oxy)-6-methyl-2-(((R)-5-oxopentan-2-yl)oxy)tetrahydro-2H-pyran-3-yl benzoate (0.150 g, 0.261 mmol) was added in THF (3 mL) dropwise via syringe. The reaction mixture was stirred for another hour then quenched with saturated aqueous ammonium chloride (1 mL), warmed to room temperature, and partitioned between ethyl acetate (10 mL) and water (5 mL). The aqueous layer was extracted with ethyl acetate (2×10 mL). The combined organic layers were dried over magnesium sulfate, filtered, and concentrated. The crude product was purified by the column chromatography (24 g silica gel, 0% to 30% acetone in hexanes) to afford I-16 (0.090 g, 50% yield). ¹H NMR (500 MHz, CDCl₃) δ 7.76-7.72 (m, 2H), 7.72-7.66 (m, 4H), 7.60-7.55 (m, 1H), 7.43-7.38 (m, 3H), 7.37-7.29 (m, 5H), 4.89 (d, J=3.6 Hz, 1H), 4.77 (s, 1H), 4.10-3.99 (m, 2H), 3.93-3.79 (m, 3H), 3.67 (tdd, J=9.1, 4.2, 2.3 Hz, 1H), 3.27 (s, 1H), 2.52 (ddd, J=16.4, 4.7, 3.0 Hz, 1H), 2.42 (ddd, J=16.4, 9.1, 0.8 Hz, 1H), 2.03 (dt, J=11.3, 3.0 Hz, 1H), 1.89 (dt, J=13.7, 3.7 Hz, 1H), 1.80-1.63 (m, 4H), 1.51 (d, J=1.5 Hz, 9H), 1.28 (dd, J=6.3, 3.2 Hz, 3H), 1.17 (d, J=6.1 Hz, 3H), 1.08 (s, 9H). Mass Analysis (ESI, +ve)=691.2 [M+H].

### Preparation of tert-butyl (6R)-6-(((2R,3R,5R,6S)-5-((tert-butyldiphenylsilyl)oxy)-3-hydroxy-6-methyltetrahydro-2H-pyran-2-yl)oxy)-3-hydroxyheptanoate (I-17)

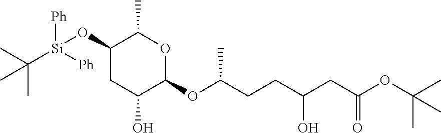 [SMILES: C[C@H](CCC(O)CC(=O)OC(C)(C)C)O[C@@H]1O[C@@H](C)[C@H](O[Si](c2ccccc2)(c2ccccc2)C(C)(C)C)C[C@H]1O]

Using the same procedure to make I-15, to a solution of I-16 (0.090 g, 0.130 mmol) in methanol (3 mL) was added 1% aq. NaOH (0.520 mL, 0.520 mmol) and after isolation and purification afforded I-17 (0.035 g, 46% yield) as a colorless paste. ¹H NMR (300 MHz, CDCl₃) δ 8.13-8.07 (m, 1H), 7.71-7.59 (m, 5H), 7.48 (dd, J=8.4, 6.9 Hz, 2H), 7.39 (dp, J=9.3, 6.1, 4.9 Hz, 5H), 4.59 (s, 1H), 4.01 (s, 1H), 3.76 (dd, J=14.1, 7.1 Hz, 2H), 3.64 (d, J=13.4 Hz, 2H), 2.49-2.32 (m, 2H), 2.17 (d, J=0.6 Hz, 2H), 1.88-1.73 (m, 2H), 1.62 (ddd, J=21.8, 11.3, 5.7 Hz, 5H), 1.47 (t, J=0.9 Hz, 9H), 1.15 (d, J=6.2 Hz, 3H), 1.11 (d, J=6.1 Hz, 3H), 1.05 (s, 9H). Mass Analysis (ESI, +ve)=587.1 [M+H].

### Preparation of tert-butyl (6R)-6-(((2R,3R,5R,6S)-3,5-dihydroxy-6-methyltetrahydro-2H-pyran-2-yl)oxy)-3-hydroxyheptanoate (9)

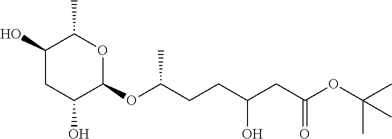 [SMILES: C[C@H](CCC(O)CC(=O)OC(C)(C)C)O[C@@H]1O[C@@H](C)[C@H](O)C[C@H]1O]

To a solution of I-17 (0.035 g, 0.060 mmol) in THF (2 mL) was added 1.0 M TBAF (0.30 mmol, 0.300 mL) in THF. The reaction mixture was stirred at room temperature under nitrogen for 18 h. The resulting mixture was concentrated and the crude product was purified by the column chromatography (24 g silica gel, 0% to 15% MeOH in DCM) to afford 9 (0.015 g, 75%) as a colorless oil. ¹H NMR (500 MHz, CDCl₃) δ 4.76-4.70 (m, 1H), 4.07-3.99 (m, 1H), 3.91-3.80 (m, 2H), 3.72-3.65 (m, 1H), 3.60 (dddd, J=11.4, 9.1, 4.6, 2.4 Hz, 1H), 3.36-3.30 (m, 1H), 2.44 (t, J=3.3 Hz, 1H), 2.39 (dd, J=8.9, 3.6 Hz, 1H), 2.13-2.07 (m, 1H), 1.85 (dddd, J=13.2, 11.1, 3.0, 2.1 Hz, 2H), 1.73-1.64 (m, 3H), 1.63-1.55 (m, 2H), 1.49 (s, 9H), 1.30 (dd, J=6.2, 3.6 Hz, 3H), 1.17 (dd, J=6.1, 1.8 Hz, 3H). Mass Analysis (ESI, +ve)=349.1 [M+H].

### Preparation of (2R,3R,5R,6S)-2-(((R,E)-7-(tert-butoxy)-7-oxohept-5-en-2-yl)oxy)-5-((tert-butyldiphenylsilyl)oxy)-6-methyltetrahydro-2H-pyran-3-yl benzoate (I-18)

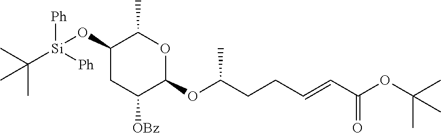

Using the same procedure to make I-09, to a solution of T-05 (0.300 g, 0.524 mmol) in DCM (5 mL) was added tert-butyl acrylate (0.230 mL, 1.572 mmol) and Grubb's M204 catalyst (0.025 g, 0.0262 mmol) and after isolation and purification afforded I-18 (0.300 g, 85% yield) as a colorless paste. ¹H NMR (500 MHz, CDCl₃) δ 7.79-7.76 (m, 2H), 7.75-7.69 (m, 4H), 7.61 (ddt, J=8.7, 7.1, 1.3 Hz, 1H), 7.46-7.41 (m, 3H), 7.40-7.33 (m, 5H), 6.98 (dt, J=15.6, 6.8 Hz, 1H), 5.88 (dt, J=15.6, 1.6 Hz, 1H), 4.92 (s, 1H), 4.80 (s, 1H), 3.94-3.84 (m, 2H), 3.71 (ddd, J=11.1, 9.1, 4.3 Hz, 1H), 2.49-2.31 (m, 3H), 2.08 (ddd, J=14.0, 11.2, 2.9 Hz, 1H), 1.94 (dt, J=13.4, 3.8 Hz, 1H), 1.87-1.78 (m, 1H), 1.71 (ddd, J=13.3, 9.2, 5.0 Hz, 1H), 1.56 (s, 9H), 1.33 (d, J=6.2 Hz, 3H), 1.21 (d, J=6.1 Hz, 3H), 1.12 (s, 9H). Mass Analysis (ESI, +ve)=673.1 [M+H].

### Preparation of tert-butyl (R,E)-6-(((2R,3R,5R,6S)-5-((tert-butyldiphenylsilyl)oxy)-3-hydroxy-6-methyltetrahydro-2H-pyran-2-yl)oxy)hept-2-enoate (I-19)

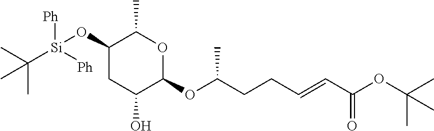 [SMILES: C[C@H](CC/C=C/C(=O)OC(C)(C)C)O[C@@H]1O[C@@H](C)[C@H](O[Si](c2ccccc2)(c2ccccc2)C(C)(C)C)C[C@H]1O]

Using the same procedure to make I-15, to a solution of I-18 (0.300 g, 0.446 mmol) in MeOH (5 mL) was added 1% aq. NaOH (3.5 mL, 0.892 mmol) and after isolation and purification I-19 (0.170 g, 67% yield) as a colorless oil. ¹H NMR (500 MHz, CDCl₃) δ 7.74-7.67 (m, 4H), 7.48-7.37 (m, 6H), 6.93 (dt, J=15.6, 6.8 Hz, 1H), 5.83 (dt, J=15.6, 1.6 Hz, 1H), 4.61 (d, J=1.6 Hz, 1H), 3.84-3.75 (m, 2H), 3.69-3.62 (m, 2H), 2.43-2.26 (m, 3H), 1.89-1.80 (m, 2H), 1.78-1.71 (m, 1H), 1.68-1.62 (m, 1H), 1.51 (s, 9H), 1.19 (d, J=6.2 Hz, 3H), 1.14 (d, J=6.1 Hz, 3H), 1.08 (s, 9H). Mass Analysis (ESI, +ve)=569.2 [M+H].

### Preparation of tert-butyl (R)-6-(((2R,3R,5R,6S)-5-((tert-butyldiphenylsilyl)oxy)-3-hydroxy-6-methyltetrahydro-2H-pyran-2-yl)oxy)heptanoate (I-20)

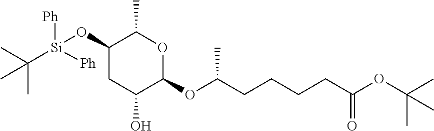 [SMILES: C[C@H](CCCCC(=O)OC(C)(C)C)O[C@@H]1O[C@@H](C)[C@H](O[Si](c2ccccc2)(c2ccccc2)C(C)(C)C)C[C@H]1O]

To a solution of I-19 (0.060 mL), which was attached with 3-way valve, in methanol (3 mL) was added Pd/C (0.015 g, 0.0105 mmol, 10% wt in wet version). The reaction mixture was first vacuumed then purged with nitrogen—this vacuumed-then-nitrogen purge procedure was repeated 3 times to ensure only nitrogen was in the flask. After the last vacuum, a hydrogen balloon was attached to the 3-way valve. The reaction mixture was stirred at room temperature under hydrogen for 18 h. The resulting mixture was filtered through a pad of Celite®. The filtrate was concentrated in vacuo to afford I-20 (0.045 g, 74% yield). ¹H NMR (500 MHz, CDCl₃) δ 7.73-7.66 (m, 4H), 7.46-7.42 (m, 2H), 7.42-7.37 (m, 4H), 4.60 (s, 1H), 3.83-3.76 (m, 2H), 3.65 (dq, J=9.0, 4.8 Hz, 2H), 3.52 (s, 2H), 2.32-2.25 (m, 2H), 1.87 (ddd, J=13.6, 10.7, 3.0 Hz, 1H), 1.83-1.77 (m, 1H), 1.71-1.61 (m, 3H), 1.50 (d, J=0.5 Hz, 2H), 1.48 (s, 9H), 1.19 (d, J=6.2 Hz, 3H), 1.12 (d, J=6.1 Hz, 3H), 1.08 (d, J=1.9 Hz, 9H). Mass Analysis (ESI, +ve)=571.2 [M+H].

### Preparation of tert-butyl (R)-6-(((2R,3R,5R,6S)-3,5-dihydroxy-6-methyltetrahydro-2H-pyran-2-yl)oxy)heptanoate (10)

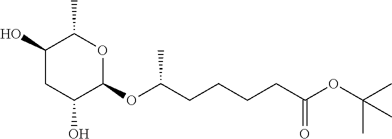 [SMILES: C[C@H](CCCCC(=O)OC(C)(C)C)O[C@@H]1O[C@@H](C)[C@H](O)C[C@H]1O]

To a solution of I-20 (0.045 g, 0.079 mmol) in THF (2 mL) was added 1.0 M TBAF (0.35 mmol, 0.350 mL) in THF. The reaction mixture was stirred at room temperature under nitrogen for 18 h. The resulting mixture was concentrated and the crude product was purified by the column chromatography (24 g silica gel, 0% to 15% MeOH in DCM) to afford 10 (0.012 g, 46% yield) as a light brown paste. ¹H NMR (500 MHz, CDCl₃) δ 4.75-4.71 (m, 1H), 3.89-3.79 (m, 3H), 3.72 (dd, J=9.2, 6.2 Hz, 1H), 3.59 (ddd, J=11.4, 9.2, 4.7 Hz, 1H), 3.38 (d, J=3.5 Hz, 1H), 2.25 (t, J=7.3 Hz, 2H), 2.15-2.07 (m, 1H), 1.86 (ddd, J=13.1, 11.3, 3.0 Hz, 2H), 1.67-1.61 (m, 3H), 1.60-1.54 (m, 2H), 1.46 (s, 9H), 1.32-1.29 (m, 3H), 1.15 (d, J=6.1 Hz, 3H). Mass Analysis (ESI, +ve)=333.2 [M+H].

### Preparation of (2R,3R,5R)-2-(((E)-6-(tert-butoxy)-6-oxohex-4-en-1-yl)oxy)-6-methyltetrahydro-2H-pyran-3,5-diyl dibenzoate (I-21)

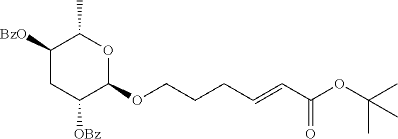

Using the same procedure to make I-07, to a solution of T-04 (0.200 g, 0.471 mmol) in DCM (5 mL) was added tert-butyl acrylate (0.205 mL, 1.41 mmol) and Grubb's M204 catalyst (0.020 g, 0.0235 mmol) and after isolation and purification afforded I-21 (0.180 g, 73% yield). ¹H NMR (300 MHz, CDCl₃) δ 8.17-8.10 (m, 2H), 8.09-8.02 (m, 2H), 7.61 (td, J=7.3, 1.7 Hz, 2H), 7.54-7.41 (m, 4H), 6.92 (dt, J=14.6, 6.9 Hz, 1H), 5.83 (d, J=16.0 Hz, 1H), 5.27-5.13 (m, 2H), 4.84 (s, 1H), 4.07 (dd, J=9.9, 6.1 Hz, 1H), 3.81 (dt, J=9.9, 6.5 Hz, 1H), 3.55 (dt, J=9.9, 6.1 Hz, 1H), 2.44 (d, J=13.9 Hz, 1H), 2.35 (q, J=7.2 Hz, 2H), 2.28-2.14 (m, 1H), 1.91-1.78 (m, 2H), 1.50 (s, 8H), 1.32 (d, J=6.2 Hz, 3H). Mass Analysis (ESI, +ve)=525.1 [M+H].

### Preparation of tert-butyl (E)-6-(((2R,3R,5R)-3,5-dihydroxy-6-methyltetrahydro-2H-pyran-2-yl)oxy)hex-2-enoate (11)

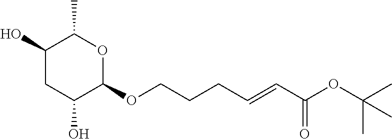 [SMILES: C[C@@H]1O[C@@H](OCCC/C=C/C(=O)OC(C)(C)C)[C@H](O)C[C@H]1O]

To a solution of I-21 (0.180 g, 0.344 mmol) in methanol (2 mL) was added K₂CO₃ (0.014 g, 0.103 mmol). The reaction mixture was stirred at room temperature under nitrogen for 18 h. The resulting mixture was concentrated and the crude product was purified by the column chromatography (24 g silica gel, 0% to 15% MeOH in DCM) to afford 11 (0.070 g, 70% yield). ¹H NMR (500 MHz, CDCl₃) δ 6.90 (dt, J=15.6, 7.0 Hz, 1H), 5.79 (dt, J=15.6, 1.6 Hz, 1H), 4.59 (d, J=1.7 Hz, 1H), 3.88 (s, 1H), 3.79-3.71 (m, 1H), 3.68-3.57 (m, 2H), 3.48 (dt, J=9.8, 5.9 Hz, 1H), 2.31 (qd, J=7.2, 1.6 Hz, 2H), 2.14-2.07 (m, 1H), 1.92-1.75 (m, 4H), 1.51 (s, 8H), 1.32 (d, J=5.9 Hz, 3H). Mass Analysis (ESI, +ve)=317.2 [M+H].

### Preparation of tert-butyl (2E)-6-{[(2S,3R,5R,6S)-3,5-bis[(tert-butyldimethylsilyl)oxy]-6-methyloxan-2-yl]oxy}-6-methylhept-2-enoate (I-22)

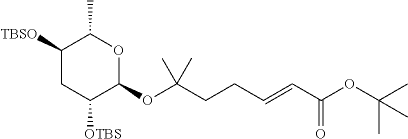 [SMILES: C[C@@H]1O[C@@H](OC(C)(C)CC/C=C/C(=O)OC(C)(C)C)[C@H](O[Si](C)(C)C(C)(C)C)C[C@H]1O[Si](C)(C)C(C)(C)C]

To a stirred solution of T-07 (0.75 mmol) in DCM (5 mL) is added tert-butyl acrylate (1.50 mmol) and Grubb's M204 catalyst (0.035 mmol). The mixture is stirred at room temperature under nitrogen for 18 h. The reaction is quenched with water (1 mL) and then partitioned between water (5 mL) and DCM (10 mL). The organic layer is washed with water (5 mL), brine (5 mL), dried over magnesium sulfate, filtered, and concentrated. The crude product is purified by the column chromatography (ethyl acetate-hexanes) to afford I-22.

### Preparation of tert-butyl (2E)-6-{[(2S,3R,5R,6S)-3,5-dihydroxy-6-methyloxan-2-yl]oxy}-6-methylhept-2-enoate (12)

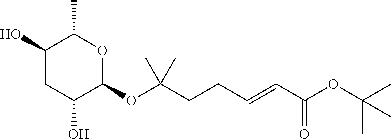 [SMILES: C[C@@H]1O[C@@H](OC(C)(C)CC/C=C/C(=O)OC(C)(C)C)[C@H](O)C[C@H]1O]

To a solution of I-22 (0.2 mmol) in THF (5 mL) is added 1.0 M TBAF (0.500 mmol, 0.500 mL) in THE. The reaction mixture is stirred at room temperature under nitrogen for 18 h. The resulting mixture is concentrated and the crude product is purified by the column chromatography (0% to 15% MeOH in DCM) to afford 12.

### Preparation of (2R,3R,5R,6S)-5-((tert-butyldiphenylsilyl)oxy)-2-(((R,E)-7-ethoxy-7-oxohept-5-en-2-yl)oxy)-6-methyltetrahydro-2H-pyran-3-yl benzoate (I-23)

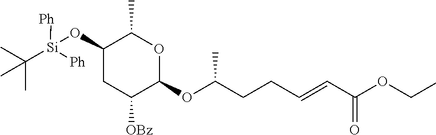

To a stirred solution of T-05 (0.420 g, 0.734 mmol) in DCM (5 mL) was added ethyl acrylate (0.160 mL, 1.470 mmol) and Grubb's M204 catalyst (0.031 g, 0.0367 mmol). The reaction mixture was stirred at room temperature under nitrogen for 18 h. The reaction mixture was quenched with water (1 mL) and then partitioned between water (5 mL) and DCM (10 mL). The organic layer was washed with water (5 mL), brine (5 mL), dried over magnesium sulfate, filtered, and concentrated. The crude product was purified by the column chromatography (40 g silica gel, 0% to 25% ethyl acetate in hexanes) to afford I-23 (0.340 g, 72% yield) as a clear oil. ¹H NMR (500 MHz, CDCl₃) δ 7.76-7.72 (m, 2H), 7.71-7.66 (m, 4H), 7.60-7.55 (m, 1H), 7.43-7.38 (m, 4H), 7.37-7.30 (m, 6H), 7.05 (dt, J=15.7, 6.8 Hz, 1H), 5.92 (dt, J=15.6, 1.6 Hz, 1H), 4.93-4.86 (m, 1H), 4.77 (s, 1H), 4.23 (q, J=7.1 Hz, 2H), 3.92-3.80 (m, 2H), 3.68 (ddd, J=11.1, 9.1, 4.3 Hz, 1H), 2.48-2.30 (m, 3H), 2.04 (ddd, J=14.0, 11.2, 3.0 Hz, 1H), 1.93-1.87 (m, 2H), 1.84-1.76 (m, 1H), 1.72-1.64 (m, 1H), 1.33 (t, J=7.1 Hz, 3H), 1.29 (d, J=6.2 Hz, 3H), 1.18 (d, J=6.1 Hz, 3H), 1.09 (s, 9H). Mass Analysis (ESI, +ve)=645.1 [M+H].

### Preparation of (R,E)-6-(((2R,3R,5R,6S)-5-((tert-butyldiphenylsilyl)oxy)-3-hydroxy-6-methyltetrahydro-2H-pyran-2-yl)oxy)hept-2-enoic acid (I-24)

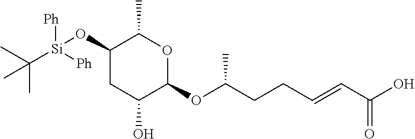 [SMILES: C[C@H](CC/C=C/C(=O)O)O[C@@H]1O[C@@H](C)[C@H](O[Si](c2ccccc2)(c2ccccc2)C(C)(C)C)C[C@H]1O]

To a stirred solution of I-23 (0.340 g, 0.527 mmol) in 1,4-dioxane (8 mL) was added lithium hydroxide (0.050 g, 2.11 mmol) and water (2 mL). The reaction mixture was heated at 60° C. under nitrogen for 18 h. The reaction mixture was cooled to room temperature and acidified with 1 M HCl to pH=5-6. The resulting mixture was extracted with 20% isopropanol in chloroform (3×10 mL). The combined organic layers were washed with brine (5 mL), dried over magnesium sulfate, filtered, and concentrated. The crude product was purified by the column chromatography (24 g silica gel, 5% to 40% ethyl acetate in hexanes with AcOH) to afford I-24 (0.220 g, 81% yield) as a white foam. ¹H NMR (500 MHz, CDCl₃) δ 7.74-7.67 (m, 4H), 7.49-7.43 (m, 2H), 7.40 (tdd, J=8.1, 6.6, 1.1 Hz, 4H), 7.16 (dt, J=15.7, 6.8 Hz, 1H), 5.93 (dt, J=15.7, 1.6 Hz, 1H), 4.62 (d, J=1.8 Hz, 1H), 3.87-3.80 (m, 1H), 3.79-3.73 (m, 1H), 3.71-3.62 (m, 2H), 2.41 (ddq, J=31.7, 15.7, 8.4 Hz, 3H), 1.90-1.81 (m, 2H), 1.80-1.73 (m, 2H), 1.72-1.63 (m, 2H), 1.19 (d, J=6.3 Hz, 3H), 1.15 (d, J=6.1 Hz, 3H), 1.08 (s, 9H). Mass Analysis (ESI, +ve)=513.2 [M+H].

### Preparation of (R,E)-6-(((2R,3R,5R,6S)-5-((tert-butyldiphenylsilyl)oxy)-3-hydroxy-6-methyltetrahydro-2H-pyran-2-yl)oxy)-1-(pyrrolidin-1-yl)hept-2-en-1-one (I-25)

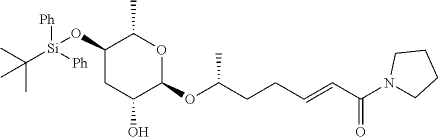 [SMILES: C[C@H](CC/C=C/C(=O)N1CCCC1)O[C@@H]1O[C@@H](C)[C@H](O[Si](c2ccccc2)(c2ccccc2)C(C)(C)C)C[C@H]1O]

To a stirred solution of I-24 (0.075 g, 0.146 mmol) in DCM (3 mL) was added DIPEA (0.130 mL, 0.732 mmol), pyrrolidine (0.040 mL, 0.439 mmol), EDCI (0.065 mL, 0.293 mmol), and HOBT (0.056 g, 0.366 mmol). The reaction mixture was stirred at room temperature under nitrogen for 18 h. The reaction mixture was quenched with saturated aqueous ammonium chloride (1 mL) then partitioned between water (5 mL) and DCM (10 mL). The aqueous layer was extracted with 20% isopropanol in chloroform (2×5 mL). The combined organic layers were dried over magnesium sulfate, filtered, and concentrated. The crude product was purified by the column chromatography (24 g silica gel, 10% to 40% acetone in hexanes) to afford I-25 (0.035 g, 40% yield) as a clear oil. ¹H NMR (500 MHz, CDCl₃) δ 7.73-7.67 (m, 4H), 7.47-7.42 (m, 2H), 7.42-7.37 (m, 4H), 7.00 (dt, J=15.1, 6.9 Hz, 1H), 6.18 (dt, J=15.1, 1.6 Hz, 1H), 4.60 (d, J=1.8 Hz, 1H), 3.85-3.76 (m, 2H), 3.69-3.62 (m, 2H), 3.55 (t, J=6.6 Hz, 3H), 2.47-2.37 (m, 1H), 2.36-2.27 (m, 1H), 1.93 (s, 3H), 1.87 (ddd, J=13.5, 10.6, 3.1 Hz, 2H), 1.83-1.74 (m, 3H), 1.65 (dq, J=8.8, 4.7, 3.7 Hz, 1H), 1.32-1.26 (m, 1H), 1.19 (d, J=6.3 Hz, 3H), 1.14 (d, J=6.1 Hz, 3H), 1.07 (s, 9H). Mass Analysis (ESI, +ve)=588.2 [M+Na].

### Preparation of (R,E)-6-(((2R,3R,5R,6S)-3,5-dihydroxy-6-methyltetrahydro-2H-pyran-2-yl)oxy)-1-(pyrrolidin-1-yl)hept-2-en-1-one (13)

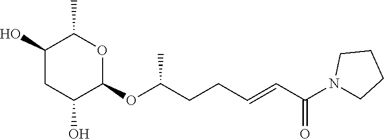 [SMILES: C[C@H](CC/C=C/C(=O)N1CCCC1)O[C@@H]1O[C@@H](C)[C@H](O)C[C@H]1O]

To a stirred solution of I-25 (0.030 g, 0.053 mmol) in THF (2 mL) was added 1.0 M TBAF (0.27 mmol, 0.270 mL) in THF. The reaction mixture was stirred at room temperature under nitrogen for 18 h. The resulting mixture was concentrated. The crude product was purified by the column chromatography (24 g silica gel, 0% to 15% MeOH in DCM) to afford 13 (0.010 g, 60% yield) as a colorless paste. ¹H NMR (500 MHz, CDCl₃) δ 7.06 (dt, J=15.2, 7.0 Hz, 1H), 6.14 (dt, J=15.2, 1.6 Hz, 1H), 4.74-4.69 (m, 1H), 3.93-3.86 (m, 1H), 3.82 (td, J=3.2, 1.7 Hz, 1H), 3.73-3.65 (m, 1H), 3.53 (t, J=6.8 Hz, 5H), 2.37 (qt, J=7.1, 1.6 Hz, 2H), 2.12 (dddd, J=13.0, 4.5, 3.3, 1.0 Hz, 1H), 2.01-1.95 (m, 4H), 1.94-1.90 (m, 1H), 1.90-1.85 (m, 2H), 1.78-1.68 (m, 2H), 1.28 (d, J=6.2 Hz, 3H), 1.18 (d, J=6.1 Hz, 3H). Mass Analysis (ESI, +ve)=328.1 [M+H].

### Preparation of ethyl (2Z,6R)-6-{[(2R,3R,5R,6S)-3,5-bis[(tert-butyldimethylsilyl)oxy]-6-methyloxan-2-yl]oxy}hept-2-enoate (I-26)

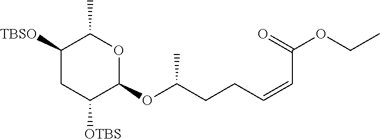 [SMILES: CCOC(=O)/C=C\CC[C@@H](C)O[C@@H]1O[C@@H](C)[C@H](O[Si](C)(C)C(C)(C)C)C[C@H]1O[Si](C)(C)C(C)(C)C]

Ethyl bis(2,2,2-trifluoroethyl)phosphonoacetate (0.25 mmol) and 18-crown-6 (0.25 mmol) are dissolved in THF (1.0 mL) and cooled to −78° C. 1.0 M KHMDS (0.25 mmol) in THF is added and the reaction is stirred at −78° C. for 30 min. Aldehyde I-03 (0.15 mmol) is added as a solution in THF (1.0 mL) and the reaction is stirred at −78° C. for 2 h and then warmed to room temperature. The mixture is quenched with aq. ammonium chloride solution (50 mL) and extracted with ethyl acetate (2×50 mL). The layers are separated and the organic layers are washed with water (2×40 mL), brine (30 mL), and the combined organic layers are dried over magnesium sulfate, filtered, and concentrate in vacuo. The crude product is purified by the column chromatography (petroleum ether-ethyl acetate) to afford I-26.

### Preparation of ethyl (2Z,6R)-6-{[(2R,3R,5R,6S)-3,5-dihydroxy-6-methyloxan-2-yl]oxy}hept-2-enoate (14)

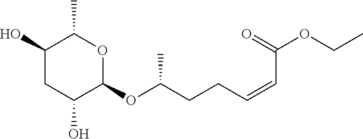 [SMILES: CCOC(=O)/C=C\CC[C@@H](C)O[C@@H]1O[C@@H](C)[C@H](O)C[C@H]1O]

To a solution of I-26 (0.2 mmol) in THF (3 mL) is added 1.0 M TBAF (0.250 mmol, 0.250 mL) in THF. The reaction mixture is stirred at room temperature under nitrogen for 18 h. The resulting mixture is concentrated and the crude product is purified by the column chromatography (0% to 15% MeOH in DCM) to afford 14.

### Preparation of ethyl 2-[(3R)-3-{[(2R,3R,5R,6S)-3,5-bis[(tert-butyldimethylsilyl)oxy]-6-methyloxan-2-yl]oxy}butyl]cyclopropane-1-carboxylate (I-27)

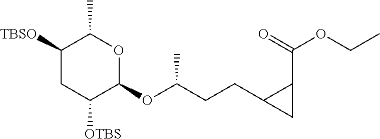 [SMILES: CCOC(=O)C1CC1CC[C@@H](C)O[C@@H]1O[C@@H](C)[C@H](O[Si](C)(C)C(C)(C)C)C[C@H]1O[Si](C)(C)C(C)(C)C]

A solution of methylene iodide (1.0 mmol) and zinc-copper couple dust (15 mg) in diethyl ether (15 mL) is stirred for 30 min and then I-26 (0.5 mmol) is added to the solution. After the reaction is refluxed for 60 hr, the mixture is poured into aq. ammonium chloride solution (50 mL). The mixture is extracted with ethyl acetate (2×50 mL). The layers are separated and the organic layers are washed with water (2×40 mL), aqueous thiosulfate (30 mL), and the combined organic layers are dried over magnesium sulfate, filtered, and concentrate in vacuo. The crude product is purified by the column chromatography (ethyl acetate-hexane) to afford I-27.

### Preparation of ethyl 2-[(3R)-3-{[(2R,3R,5R,6S)-3,5-dihydroxy-6-methyloxan-2-yl]oxy}butyl]cyclopropane-1-carboxylate (15)

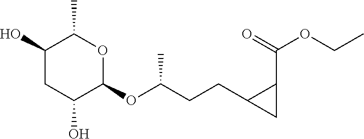 [SMILES: CCOC(=O)C1CC1CC[C@@H](C)O[C@@H]1O[C@@H](C)[C@H](O)C[C@H]1O]

To a solution of I-27 (0.1 mmol) in THF (3 mL) is added 1.0 M TBAF (0.150 mmol, 0.150 mL) in THE. The reaction mixture is stirred at room temperature under nitrogen for 18 h. The resulting mixture is concentrated and the crude product is purified by the column chromatography (0% to 15% MeOH in DCM) to afford 15.

Using the protocols described above, compounds 16-56, which are exemplified in Table 3, are prepared.

TABLE 3
Ascaroside derivative

| No. | Structure |
|---|---|
| 1 | 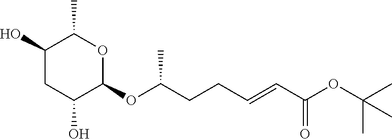 [SMILES: C[C@H](CC/C=C/C(=O)OC(C)(C)C)O[C@@H]1O[C@@H](C)[C@H](O)C[C@H]1O] |
| 2 | 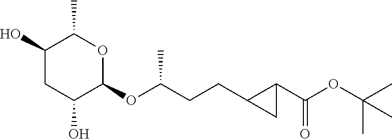 [SMILES: C[C@H](CCC1CC1C(=O)OC(C)(C)C)O[C@@H]1O[C@@H](C)[C@H](O)C[C@H]1O] |
| 3 | 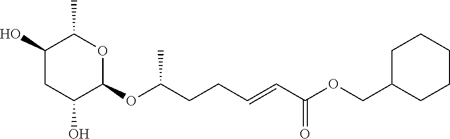 [SMILES: C[C@H](CC/C=C/C(=O)OCC1CCCCC1)O[C@@H]1O[C@@H](C)[C@H](O)C[C@H]1O] |
| 4 | 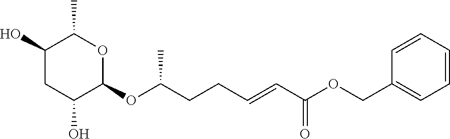 [SMILES: C[C@H](CC/C=C/C(=O)OCc1ccccc1)O[C@@H]1O[C@@H](C)[C@H](O)C[C@H]1O] |
| 5 | 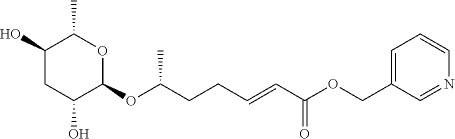 [SMILES: C[C@H](CC/C=C/C(=O)OCc1cccnc1)O[C@@H]1O[C@@H](C)[C@H](O)C[C@H]1O] |
| 6 | 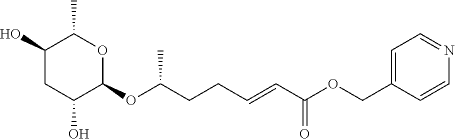 [SMILES: C[C@H](CC/C=C/C(=O)OCc1ccncc1)O[C@@H]1O[C@@H](C)[C@H](O)C[C@H]1O] |
| 7 | 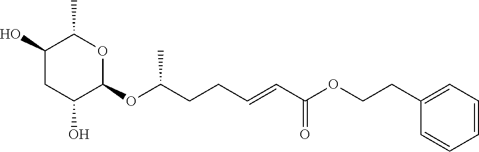 [SMILES: C[C@H](CC/C=C/C(=O)OCCc1ccccc1)O[C@@H]1O[C@@H](C)[C@H](O)C[C@H]1O] |
| 8 | 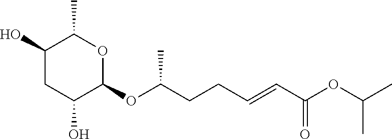 [SMILES: CC(C)OC(=O)/C=C/CC[C@@H](C)O[C@@H]1O[C@@H](C)[C@H](O)C[C@H]1O] |
| 9 | 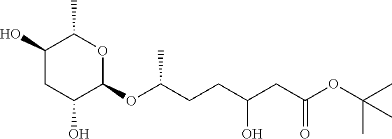 [SMILES: C[C@H](CCC(O)CC(=O)OC(C)(C)C)O[C@@H]1O[C@@H](C)[C@H](O)C[C@H]1O] |
| 10 | 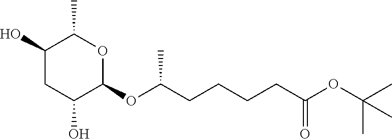 [SMILES: C[C@H](CCCCC(=O)OC(C)(C)C)O[C@@H]1O[C@@H](C)[C@H](O)C[C@H]1O] |
| 11 | 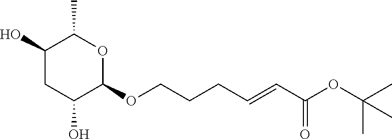 [SMILES: C[C@@H]1O[C@@H](OCCC/C=C/C(=O)OC(C)(C)C)[C@H](O)C[C@H]1O] |
| 12 | 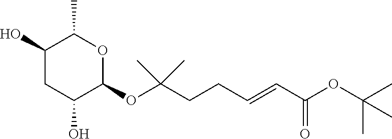 [SMILES: C[C@@H]1O[C@@H](OC(C)(C)CC/C=C/C(=O)OC(C)(C)C)[C@H](O)C[C@H]1O] |
| 13 | 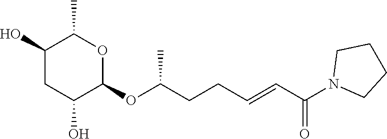 [SMILES: C[C@H](CC/C=C/C(=O)N1CCCC1)O[C@@H]1O[C@@H](C)[C@H](O)C[C@H]1O] |
| 14 | 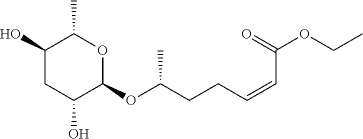 [SMILES: CCOC(=O)/C=C\CC[C@@H](C)O[C@@H]1O[C@@H](C)[C@H](O)C[C@H]1O] |
| 15 | 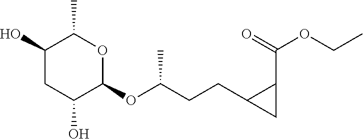 [SMILES: CCOC(=O)C1CC1CC[C@@H](C)O[C@@H]1O[C@@H](C)[C@H](O)C[C@H]1O] |
| 16 | 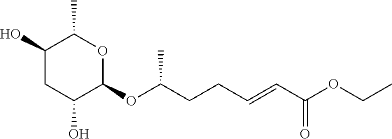 [SMILES: CCOC(=O)/C=C/CC[C@@H](C)O[C@@H]1O[C@@H](C)[C@H](O)C[C@H]1O] |
| 17 | 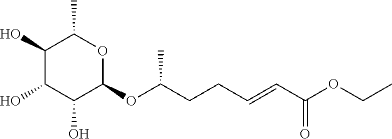 [SMILES: CCOC(=O)/C=C/CC[C@@H](C)O[C@@H]1O[C@@H](C)[C@H](O)[C@@H](O)[C@H]1O] |
| 18 | 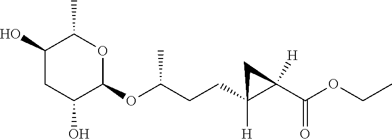 [SMILES: CCOC(=O)[C@@H]1C[C@H]1CC[C@@H](C)O[C@@H]1O[C@@H](C)[C@H](O)C[C@H]1O] |
| 19 | 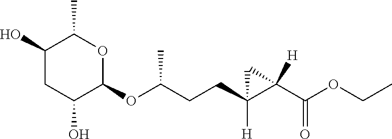 [SMILES: CCOC(=O)[C@H]1C[C@@H]1CC[C@@H](C)O[C@@H]1O[C@@H](C)[C@H](O)C[C@H]1O] |
| 20 | 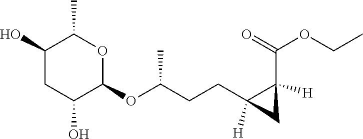 [SMILES: CCOC(=O)[C@H]1C[C@H]1CC[C@@H](C)O[C@@H]1O[C@@H](C)[C@H](O)C[C@H]1O] |
| 21 | 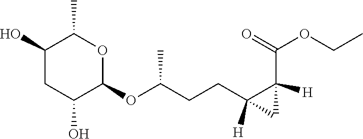 [SMILES: CCOC(=O)[C@@H]1C[C@@H]1CC[C@@H](C)O[C@@H]1O[C@@H](C)[C@H](O)C[C@H]1O] |
| 22 | 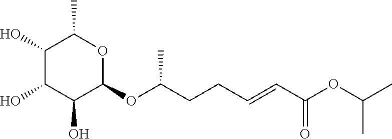 [SMILES: CC(C)OC(=O)/C=C/CC[C@@H](C)O[C@@H]1O[C@@H](C)[C@@H](O)[C@@H](O)[C@@H]1O] |
| 23 | 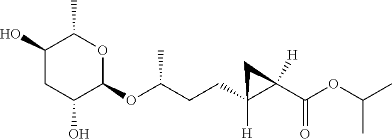 [SMILES: CC(C)OC(=O)[C@@H]1C[C@H]1CC[C@@H](C)O[C@@H]1O[C@@H](C)[C@H](O)C[C@H]1O] |
| 24 | 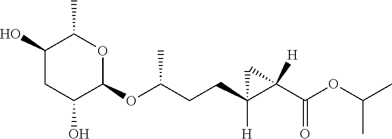 [SMILES: CC(C)OC(=O)[C@H]1C[C@@H]1CC[C@@H](C)O[C@@H]1O[C@@H](C)[C@H](O)C[C@H]1O] |
| 25 | 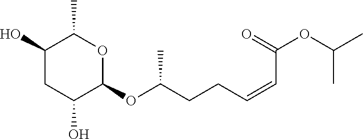 [SMILES: CC(C)OC(=O)/C=C\CC[C@@H](C)O[C@@H]1O[C@@H](C)[C@H](O)C[C@H]1O] |
| 26 | 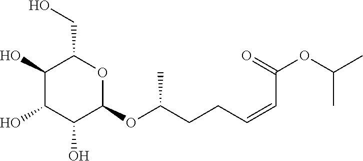 [SMILES: CC(C)OC(=O)/C=C\CC[C@@H](C)O[C@@H]1O[C@@H](CO)[C@H](O)[C@@H](O)[C@H]1O] |
| 27 | 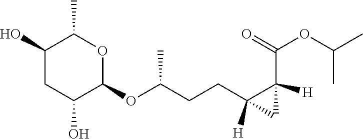 [SMILES: CC(C)OC(=O)[C@@H]1C[C@@H]1CC[C@@H](C)O[C@@H]1O[C@@H](C)[C@H](O)C[C@H]1O] |
| 28 | 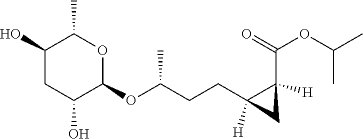 [SMILES: CC(C)OC(=O)[C@H]1C[C@H]1CC[C@@H](C)O[C@@H]1O[C@@H](C)[C@H](O)C[C@H]1O] |
| 29 | 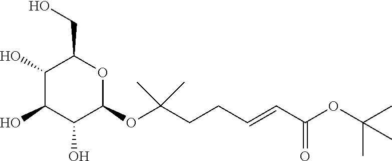 [SMILES: CC(C)(C)OC(=O)/C=C/CCC(C)(C)O[C@@H]1O[C@H](CO)[C@@H](O)[C@H](O)[C@H]1O] |
| 30 | 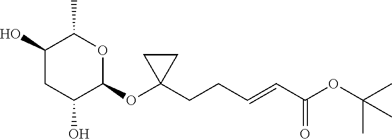 [SMILES: C[C@@H]1O[C@@H](OC2(CC/C=C/C(=O)OC(C)(C)C)CC2)[C@H](O)C[C@H]1O] |
| 31 | 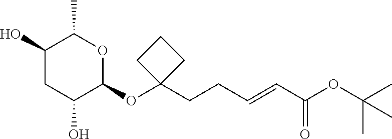 [SMILES: C[C@@H]1O[C@@H](OC2(CC/C=C/C(=O)OC(C)(C)C)CCC2)[C@H](O)C[C@H]1O] |
| 32 | 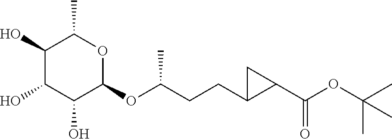 [SMILES: C[C@H](CCC1CC1C(=O)OC(C)(C)C)O[C@@H]1O[C@@H](C)[C@H](O)[C@@H](O)[C@H]1O] |
| 33 | 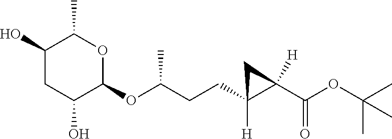 [SMILES: C[C@H](CC[C@@H]1C[C@H]1C(=O)OC(C)(C)C)O[C@@H]1O[C@@H](C)[C@H](O)C[C@H]1O] |
| 34 | 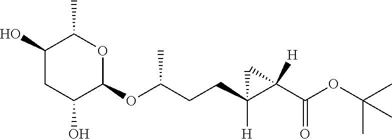 [SMILES: C[C@H](CC[C@H]1C[C@@H]1C(=O)OC(C)(C)C)O[C@@H]1O[C@@H](C)[C@H](O)C[C@H]1O] |
| 35 | 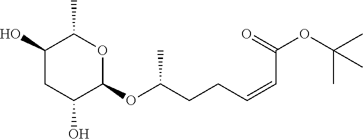 [SMILES: C[C@H](CC/C=C\C(=O)OC(C)(C)C)O[C@@H]1O[C@@H](C)[C@H](O)C[C@H]1O] |
| 36 | 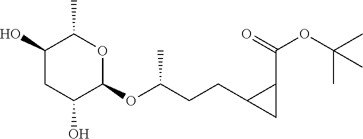 [SMILES: C[C@H](CCC1CC1C(=O)OC(C)(C)C)O[C@@H]1O[C@@H](C)[C@H](O)C[C@H]1O] |
| 37 | 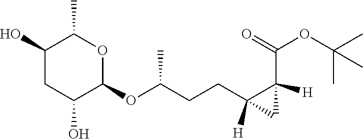 [SMILES: C[C@H](CC[C@H]1C[C@H]1C(=O)OC(C)(C)C)O[C@@H]1O[C@@H](C)[C@H](O)C[C@H]1O] |
| 38 | 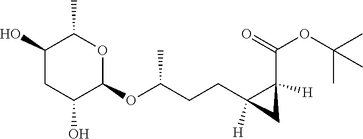 [SMILES: C[C@H](CC[C@@H]1C[C@@H]1C(=O)OC(C)(C)C)O[C@@H]1O[C@@H](C)[C@H](O)C[C@H]1O] |
| 39 | 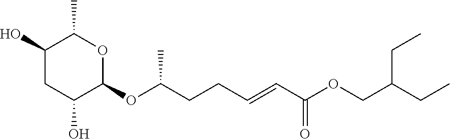 [SMILES: CCC(CC)COC(=O)/C=C/CC[C@@H](C)O[C@@H]1O[C@@H](C)[C@H](O)C[C@H]1O] |
| 40 | 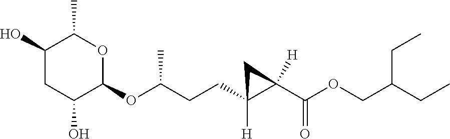 [SMILES: CCC(CC)COC(=O)[C@@H]1C[C@H]1CC[C@@H](C)O[C@@H]1O[C@@H](C)[C@H](O)C[C@H]1O] |
| 41 | 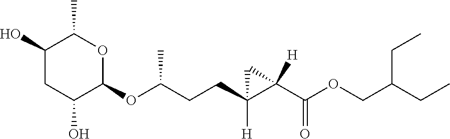 [SMILES: CCC(CC)COC(=O)[C@H]1C[C@@H]1CC[C@@H](C)O[C@@H]1O[C@@H](C)[C@H](O)C[C@H]1O] |
| 42 | 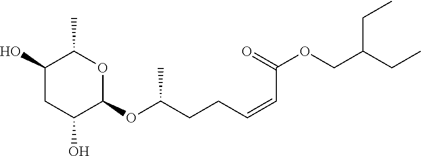 [SMILES: CCC(CC)COC(=O)/C=C\CC[C@@H](C)O[C@@H]1O[C@@H](C)[C@H](O)C[C@H]1O] |
| 43 | 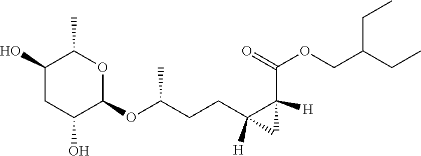 [SMILES: CCC(CC)COC(=O)[C@@H]1C[C@@H]1CC[C@@H](C)O[C@@H]1O[C@@H](C)[C@H](O)C[C@H]1O] |
| 44 | 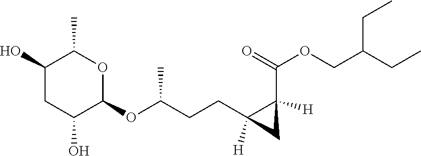 [SMILES: CCC(CC)COC(=O)[C@H]1C[C@H]1CC[C@@H](C)O[C@@H]1O[C@@H](C)[C@H](O)C[C@H]1O] |
| 45 | 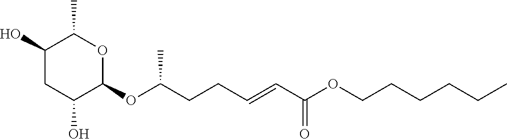 [SMILES: CCCCCCOC(=O)/C=C/CC[C@@H](C)O[C@@H]1O[C@@H](C)[C@H](O)C[C@H]1O] |
| 46 | 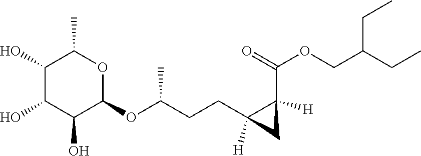 [SMILES: CCC(CC)COC(=O)[C@H]1C[C@H]1CC[C@@H](C)O[C@@H]1O[C@@H](C)[C@@H](O)[C@@H](O)[C@@H]1O] |
| 47 | 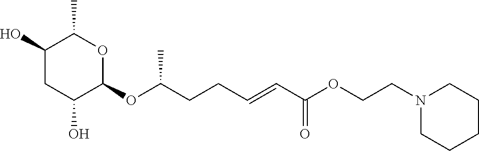 [SMILES: C[C@H](CC/C=C/C(=O)OCCN1CCCCC1)O[C@@H]1O[C@@H](C)[C@H](O)C[C@H]1O] |
| 48 | 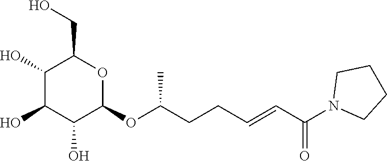 [SMILES: C[C@H](CC/C=C/C(=O)N1CCCC1)O[C@@H]1O[C@H](CO)[C@@H](O)[C@H](O)[C@H]1O] |
| 49 | 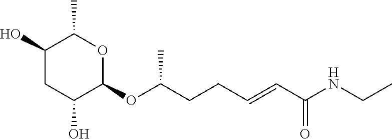 [SMILES: CCNC(=O)/C=C/CC[C@@H](C)O[C@@H]1O[C@@H](C)[C@H](O)C[C@H]1O] |
| 50 | 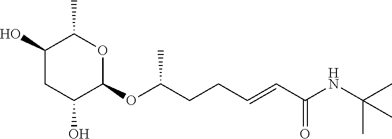 [SMILES: C[C@H](CC/C=C/C(=O)NC(C)(C)C)O[C@@H]1O[C@@H](C)[C@H](O)C[C@H]1O] |
| 51 | 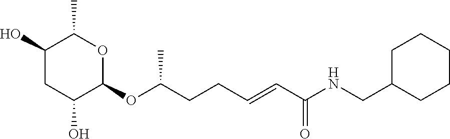 [SMILES: C[C@H](CC/C=C/C(=O)NCC1CCCCC1)O[C@@H]1O[C@@H](C)[C@H](O)C[C@H]1O] |
| 52 | 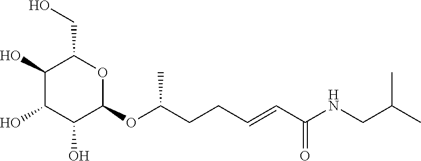 [SMILES: CC(C)CNC(=O)/C=C/CC[C@@H](C)O[C@@H]1O[C@@H](CO)[C@H](O)[C@@H](O)[C@H]1O] |
| 53 | 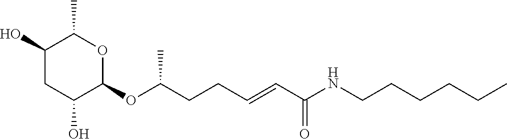 [SMILES: CCCCCCNC(=O)/C=C/CC[C@@H](C)O[C@@H]1O[C@@H](C)[C@H](O)C[C@H]1O] |
| 54 | 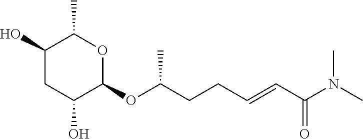 [SMILES: C[C@H](CC/C=C/C(=O)N(C)C)O[C@@H]1O[C@@H](C)[C@H](O)C[C@H]1O] |
| 55 | 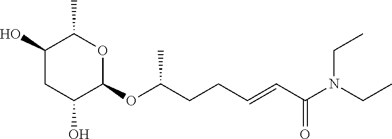 [SMILES: CCN(CC)C(=O)/C=C/CC[C@@H](C)O[C@@H]1O[C@@H](C)[C@H](O)C[C@H]1O] |
| 56 | 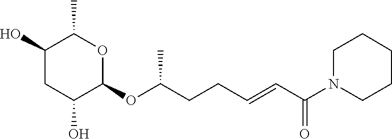 [SMILES: C[C@H](CC/C=C/C(=O)N1CCCCC1)O[C@@H]1O[C@@H](C)[C@H](O)C[C@H]1O] |

### Compositions and Methods

Described herein are examples of treatment of microbiota using one or more ascarosides and/or ascaroside derivatives.

It has become possible to perform rapid and relatively inexpensive analyses of a subject's microbiome (a “microbiome profile”) from a single specimen or swab, using technology based on identifying microorganism-specific DNA or RNA sequences. Such a microbiome profile may readily be compared with profiles based on large populations of healthy individuals, or with those based on populations of individuals who suffer from a particular disease or disease risk. Such a comparison may suggest that an individual has, or is at risk for, such a disease, based upon the individual's microbiome profile, as has been suggested in the scientific literature. Indicators of disease or disease risk on a microbiome profile may include, but are not limited to, assessments of the absolute or relative abundance of specific microorganism strains, species, genera, or larger taxonomic units, or the diversity of the measured microbiome population. These profiles may also or alternatively be used to track the therapeutic effect of one or more compositions as described herein.

### Example 1

In one variation, an animal model (e.g., mouse) was treated with control vehicle and compared with mice treated with one or more ascarosides to assess the effect on their gut microbiome. Fecal pellets were collected for genotyping and identification of bacterial strains (and distribution and quantity of bacterial strains) from mice treated with control vehicle and compared to those treated with ascaroside pheromones, to identify modification of the microbiome.

### Example 2

0.001-10 mM of the compound of Formula I is administered by oral gavage to C56B16 or BalbC mice once a day, for up to 21 days, and fecal pellet were collected prior to the administration of the compound, daily, and after completion of the administration of the compound. Fecal pellets were then stored at −20 C or −4 C for storage until analysis. Sequencing of preparations of the fecal pellet were performed and the following will be reported: The identification of species and count noted the diversity of the bacterial strains, outlying populations, and note the prevalence of generally beneficial distributions.

### Example 3

0.001-10 mM of the compound of Formula I was administered by intraperitoneal injection to C56B16 or BalbC mice once a day, for up to 21 days, and fecal pellet were collected prior to the administration of the compound, daily, and after completion of the administration of the compound. Fecal pellets were then stored at −20 C or −4 C for storage until analysis. Sequencing of preparations of the fecal pellet was performed and the following reported: the identification of species and count to note the diversity of the bacterial strains, outlying populations, and note the prevalence of generally beneficial distributions.

In the methods described for each of these examples, C57BL6 mice at 6 weeks of age were allowed to be appropriately acclimated before the start of the change in diet and treatments. For Experiment 1 (Example 1), thirty-two mice were evenly distributed into four groups with Group 1 as the control diet, no treatment, Group 2 as the high-fat diet, treat with DMSO control, Group 3 as the high-fat diet, treated with 12.5 μg of Ascr #7/mouse per injection (0.5 mg/kg dose assuming a 25 gm mouse), Group 4 as the high-fat diet, treated with 4.1 μg Ascr #9/mouse per injection (0.164 mg/kg dose assuming a 25 gm mouse). Ascr #7 and Ascr #9 compounds were dissolved in dimethyl sulfoxide (DMSO) to make master stocks of 10 μg/uL of Ascr #7 and 3.3 μg/uL of Ascr #9 and 12.5 μL of each master stocks and DMSO only were aliquoted in glass vial for each day of injections. Mice in Group 2, 3 and 4 were intraperitoneally (IP) injected with DMSO, Ascr #7, or Ascr #9, respectively three times a week for 31 Days, when stool was collected and compared to stool collected at the start of the experiment. For each day of injection, 987.5 of ultra-pure water is added to each tube to make DMSO control, 0.125 μg/uL of Ascr #7 and 0.041 of Ascr #9 working stocks and 100 μL of the each working stocks were IP injected into mice from Group 2, 3 and 4, respectively.

For Experiment 2 (Example 2), eighteen mice were equally divided in Group 5 as the treated of DMSO control and 6 as the treated with 12.5 μg of Ascr #7/mouse per injection (0.5 mg/kg dose assuming a 25 gm mouse). To make the master stock, Ascr #7 was dissolved in DMSO to make 10 μg/μL and 12.5 μL of DMSO only and Ascr #7 master stock were aliquoted into glass vials for each day of injection. Mice from Group 5 and 6 were IP injected three times a week for 3 weeks and one day for a total of 10 injections. On each day of IP injection, 987.5 μL of ultra-pure water is added to each master stocks to make DMSO only and 0.125 μg/μL of Ascr #7 working stocks and 100 μL of each was IP injected into mice from Group 5 and 6, respectively.

For Experiment 3, ten mice were assigned Group 7 to be treated with DMSO control. Eight mice were assigned to Group 8 to be treated with 12.5 μg of Ascr #7/mouse per injection (0.5 mg/kg dose assuming a 25 gm mouse). Nine mice were assigned to Group 9 to be treated with 12.5 μg of Compound 1/mouse per injection (0.5 mg/kg dose assuming a 25 gm mouse). To make the master stocks, Ascr #7 and Compound 1 were dissolved in DMSO to make 10 μg/uL of the Ascr #7 and Compound 1 master stocks and 12.5 μL of each master stocks were aliquoted into glass vials for each day of injection. Mice from each group were IP injected three times a week for 3 weeks and one day for a total of 10 injections. On each day of IP injection, 987.5 μL of ultra-pure water is added to each master stocks to make 0.125 μg/uL of Ascr #7 and Compound 1 working stocks and 100 μL of each was IP injected into mice. As will be described in greater detail below, Compound 1 refers to the ascaroside derisive:

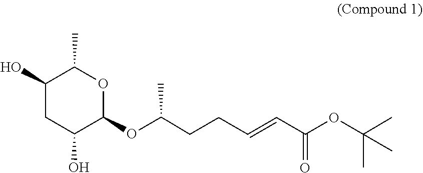 [SMILES: C[C@H](CC/C=C/C(=O)OC(C)(C)C)O[C@@H]1O[C@@H](C)[C@H](O)C[C@H]1O]

DNA was extracted using the Qiagen MagAttract PowerSoil DNA KF kit (Formerly MO Bio PowerSoil DNA Kit) using a KingFisher robot. DNA quality was evaluated visually via gel electrophoresis and quantified using a Qubit 3.0 fluorometer (Thermo-Fischer, Waltham, Mass., USA). Libraries were prepared using an Illumina Nextera library preparation kit using an in-house protocol (Illumina, San Diego, Calif., USA).

Paired-end sequencing (150 bp×2) was done on a NextSeq 500 in medium-output mode. Shotgun metagenomic sequence reads were processed with the Sunbeam pipeline. Initial quality evaluation was done using FastQC v0.11.5. Processing took part in four steps: adapter removal, read trimming, low-complexity reads removal, and host-sequence read removal. Adapter removal was done using cutadapt v2.6. Trimming was done with Trimmomatic v0.36 using custom parameters (LEADING:3 TRAILING:3 SLIDINGWINDOW:4:15 MINLEN:36). Low-complexity sequences were detected with Komplexity v0.3.6.

The remaining reads were taxonomically classified using Kraken2 with the MiniKraken2_v1 database. For functional profiling, high-quality (filtered) reads were aligned against the SEED database via translated homology search and annotated to Subsystems, or functional levels, 1-3 using Super-Focus.

Metric multidimensional scaling analysis was used to visualize microbiome similarities. Permutational analysis of variance (PERMANOVA) was used to formally test for the significance of overall microbiome differences.

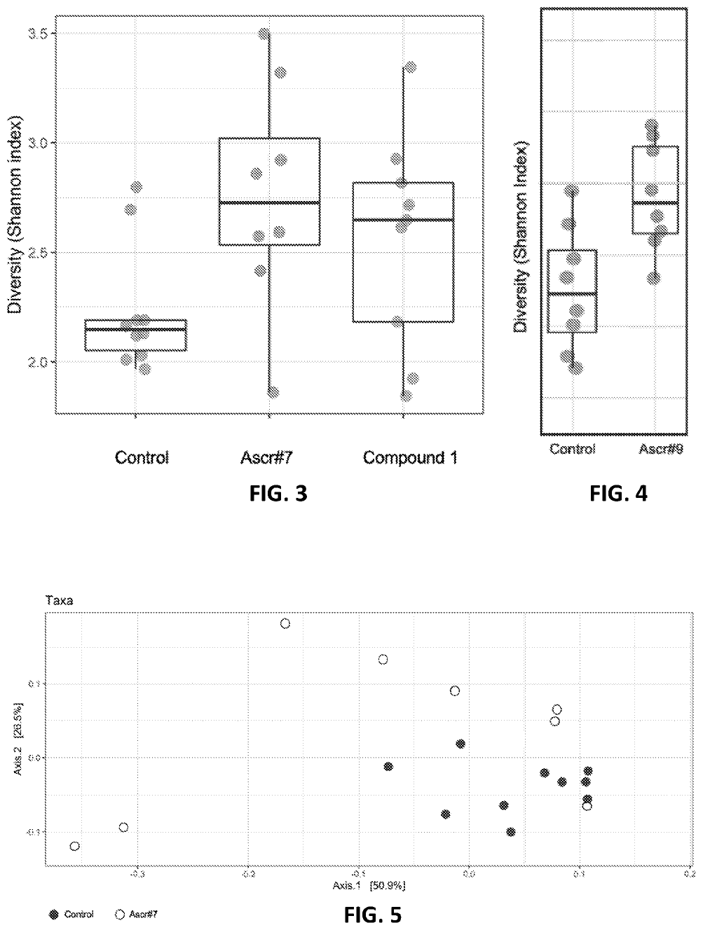

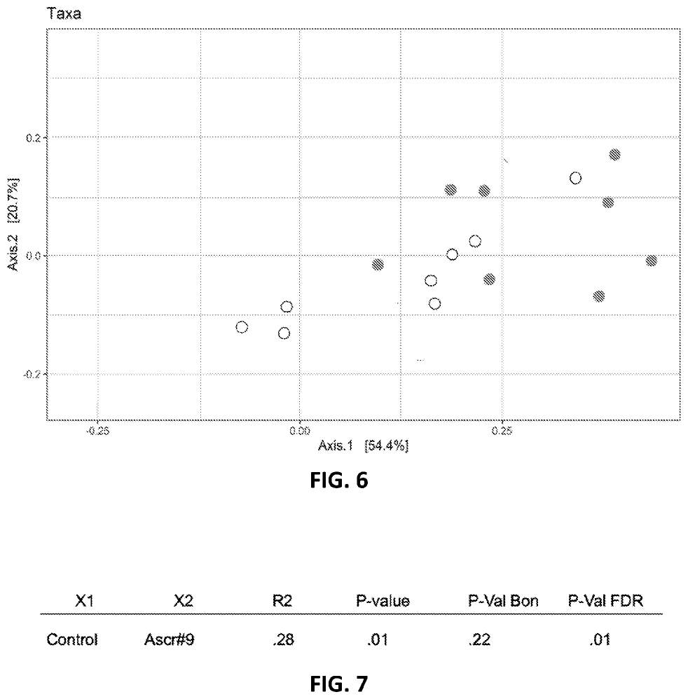

C57BL6 Mice at 6 weeks of life were injected IP with Ascr #7, Ascr #9 or Compound 1 three times per week for 4 weeks or 12 weeks. Stools were collected before and after experiment and stored frozen at −80° C. until sequenced. Alpha diversity was calculated from taxonomic profiles using Shannon's diversity index (see, e.g., FIGS. 3 and 4, discussed below). Beta Diversity (e.g., pairwise differences in taxonomic profiles) was examined by differential abundance analysis (see, e.g., FIG. 5 and FIG. 6, discussed below). Negative binomial models (DESEq2 R package) were ran for differential abundance testing of taxonomic and Subsystem level 3 features. P values of were calculated with Likelihood Ratio Tests. Post-hoc pairwise comparisons were conducted with Wald tests. Nominal P values were corrected for multiple comparisons with the Benajmini-Hochberg procedure; adjusted P values were considered significant if they were below 0.1. See, for example, the table of FIG. 7.

### Diversity of the Gut Microbiota

In general, the methods and compositions described herein generally increased the diversity of the microbiome. Typically, the application of at least one ascaroside modified the composition of the microbiome as compared to control animals. Based on experiments similar to those above including Experiments 1-3), the application of an ascaroside to a patient (e.g., a human or animal having a microbiome) resulted in a significant increase in the diversity of the microbiome. As described herein and known in the art, an ascaroside has a formulation of Formula I or a sub-set of these, e.g., a naturally-occurring ascaroside, a Ascaroside derivative, or an ascaroside of Formula II:

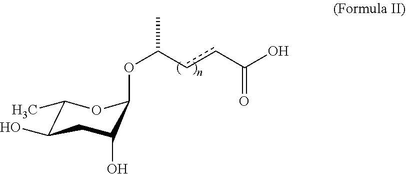

Where: [symbol] represents a double or single bond, and ( )ₙ represents (CH₂)ₙ where n may be 1-12.

Any of the ascaroside described herein, individually and in various combinations, may increase the diversity of the patient's microbiome. For example, FIG. 3 illustrates the results of treatment with the Ascr #7.

As shown in FIG. 3, treatment with the ascaroside (e.g., Ascr #7), as compared to control, resulted in a substantial increase in diversity of the subject's gut microbiota. Similar results were seen, or are anticipated to be seen, with each of the ascarosides, an in particular the ascarosides having Formula II (for each n between 1-12). For example, FIG. 4 illustrates another example of the effect of a treatment with ascaroside on a patient's gut microbiota. In FIG. 4, the diversity of the gut microbiota was increased by treatment with an ascaroside; in this example, the ascaroside was Ascr #9.

FIGS. 5 and 6 illustrate beta diversity graphs for data from Ascr #7 and Ascr #9, as described above.

Thus, any of the methods and compositions described herein may increase the diversity of gut microbiota, which may be beneficial to the patient, and may enhance overall patient gut health. Although examples of Ascr #7 and Ascr #9 are shown above, a method of treating a subject to selectively alter the composition of the subject's gut microbiome may include administering an amount of at least one ascaroside having the formulation of Formula I or Formula II. In some variations these compositions may combinations or mixtures of Ascr #7 and one or more additional ascaroside. For example, a composition may include Ascr #7 and any other ascaroside. Thus, the composition may include 2 or more ascaroside (e.g., 3 or more ascarosides, 4 or more ascaroside, etc.), including a salt form or any of these ascarosides. In general, the ascaroside is isolated and/or synthesized. As will be described herein, the dose form may be in any appropriate form, including in particular an oral dose form. The dose form may be a time release dose form.

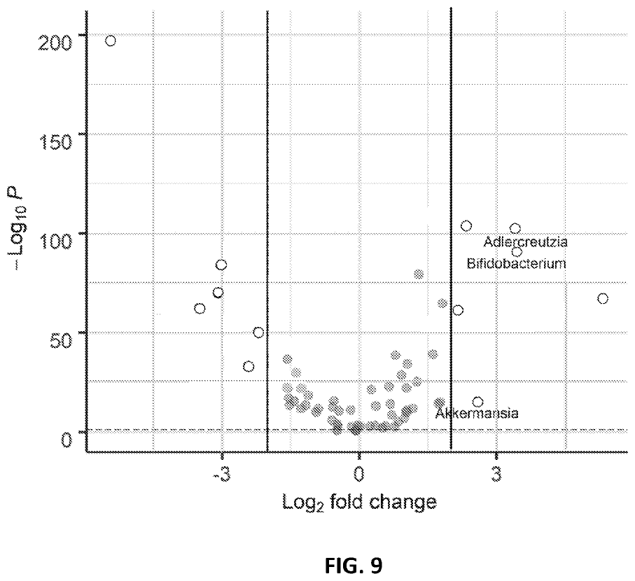

The methods and compositions described herein may also be configured to enhance and/or increase the presence of one or more species of gut microbiota, including for example, Bifidobacterium (e.g., Bifidobacterium catenulatum), Akkermansia and Adlercreutzia. This is illustrated in FIG. 9, showing the overall microbiome differences between treated (with one or more ascaroside) and untreated (control) mice. For example, in FIG. 9, the stools of mice from experiment 1 were compared between control and treatment following 31 days of treatment. The results show that a significant increase (as seen in the permutational analysis of variance analysis) in the marked Bifidobacterium, Akkermansia and Adlercreutzia within the gut microbiota.

Increasingly, evidence is accumulating which shows beneficial effects of supplementation with Bifidobacteria for the improvement of human health conditions ranging from protection against infection to different extra- and intra-intestinal positive effects. Moreover, Bifidobacteria have been associated with the production of a number of potentially health promoting metabolites including short chain fatty acids, conjugated linoleic acid and bacteriocins.

Bifidobacteria are normal inhabitants of the gut mictoriota belonging to the Actinobacteria phylum. After the depletion of oxygen by facultative anaerobes, bifidobacterial populations are the most abundant genus present in the healthy infant gut. During adulthood the levels decrease considerably but remain relatively stable; decreasing again in old age and in particular in certain disease states (e.g., cancer). Certain strains of Bifidobacterium are used as probiotics. Bifidobacteria are one of the most abundant genera present in the healthy infant gut and represent a significant portion of the microbiota throughout a healthy adult life, playing an important role in gut homeostasis and health. However, during late adulthood and within several diseases, the levels of Bifidobacterium and its species diversity decrease. This decrease may play a role in certain disorders, including (but not limited to) irritable bowel syndrome.

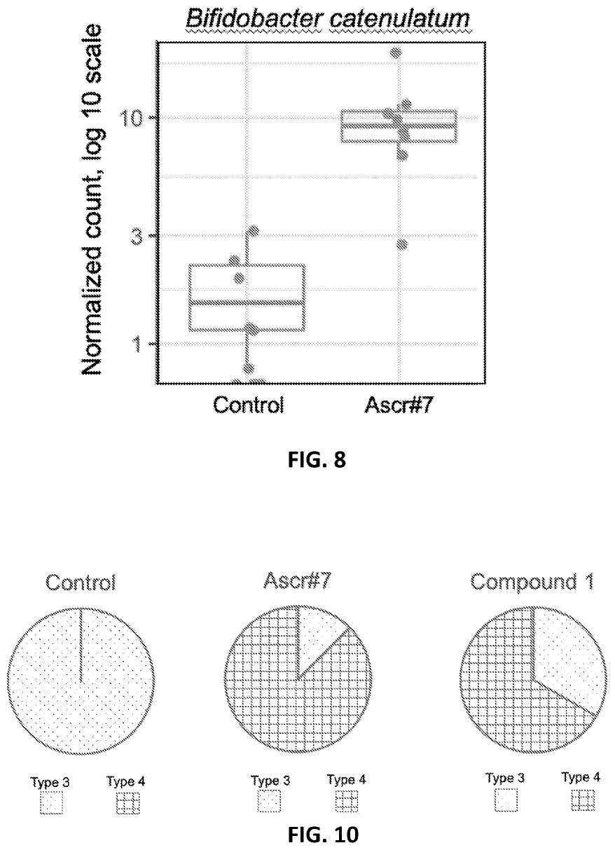

The methods and compositions described herein may enhance, increase and/or protect the population of Bifidobacterium, including Bifidobacterium catenulatum. For example, FIG. 8 illustrates the effect of ascaroside on a subject's population of Bifidobacterium catenulatum as compared to control. As described above, subjects (e.g., mice) were treated with ascaroside (in FIG. 8, specifically, the particular ascaroside used was Ascr #7, although similar results were seen with all ascarosides tested). In this example, following treatment with ascaroside, the amount of Bifidobacterium catenulatum present in the gut microbiota increased significantly.

The methods and compositions described herein may enhance, increase and/or protect the population of Adlercreutzia, including Adlercreutzia equolifasciens. Adlercreutzia has been implicated in the production of antioxidants, as well as converting—epigallocatechin (−EGC), −epicatechin (−EC), −catechin (−C), and +catechin (+C) into their corresponding metabolites, and catalyzing these metabolites by hydroxylation reactions and produce a range of phenyl-valerolactones metabolites presumed to have metabolic bioactivities. These bioactivities may be helpful in treatment of, e.g., menopausal symptoms such as hot flashes and benign prostatic hyperplasia. As described above, subjects were treated with ascaroside (including but not limited to Ascr #7 and Ascr #9) showed an increase in the overall amount of Adlercreutzia in the gut microbiota.

The methods and compositions described herein may enhance, increase and/or protect the population of Akkermansia, including Akkermansia muciniphila. Akkermansia muciniphila can reside in the human intestinal tract and is currently being studied for its effects on human metabolism. Recently performed studies in rodents have indicated that Akkermansia muciniphila in the intestinal tract may contribute to a reduction in obesity. Subjects treated with ascaroside (including but not limited to Ascr #7 and Ascr #9) showed an increase in the overall amount of Akkermansia in the gut microbiota.

The human gastrointestinal (GI) tract contains an abundant and diverse microbial community that gathers more than 100 trillion microorganisms. The density of bacterial cells in the colon has been estimated at 10¹¹ to 10¹² per milliliter which makes the colon one of the most densely populated microbial habitats known on earth. The gut microbiome encodes over 3 million genes producing thousands of metabolites, whereas the human genome consists of approximately 23,000 genes. Gut bacteria are key regulators of digestion along the gastrointestinal tract; commensal bacteria play an important role in the extraction, synthesis, and absorption of many nutrients and metabolites, including bile acids, lipids, amino acids, vitamins, and short-chain fatty acids (SCFAs). Gut microbiota have a crucial immune function against pathogenic bacteria colonization inhibiting their growth, consuming available nutrients and/or producing bacteriocins. Gut microbiota also prevent bacteria invasion by maintaining the intestinal epithelium integrity. Microorganisms prevent pathogenic colonization by many competition processes: nutrient metabolism, pH modification, antimicrobial peptide secretions, and effects on cell signaling pathways. Moreover, recent studies have identified a critical role for commensal bacteria and their products in regulating the development, homeostasis, and function of innate and adaptive immune cells. It is paradoxical to note that the gut microbiota functions are highly preserved between individuals, whereas each individual's gut microbiota are characterized by a specific combination of bacterial species due to inter-individual and intra-individual variations throughout human life.

Although subjects, and in particular human subjects may have variations in gut microbiota, it is becoming clear that, in general, the overall diversity of the gut microbiota, even in the presence of variations within and between individuals, appears to be important in preserving health. In addition, certain types of gut microbiota may be of particular use. Thus, the methods and compositions described herein, specifically methods of using ascaroside and ascaroside compositions, enhance both the diversity of gut microbiota and increase to overall amount of, e.g., Bifidobacterium, Akkermansia and Adlercreutzia. Thus, these methods and compositions enhance the strong relationship between gut microbiota diversity and health.

Any of the methods and compositions described herein may be used in conjunction with one or more antibiotic, including in before, during or after a course of antibiotics, in order to reduce the impact of the one or more antibiotic on the gut microbiota. For example, the methods and compositions described herein may include concurrent delivery of one or more ascaroside as described herein and one or more antibiotic, such as one or more of the Penicillins, Tetracyclines, Cephalosporins, Quinolones, Lincomycins, Macrolides, Sulfonamides, Glycopeptides, Aminoglycosides, and/or Carbapenems. For example, the antibiotic may include one or more of amoxicillin, doxycycline, cephalexin, ciprofloxacin, clindamycin, metronidazole, azithromycin, sulfamethoxazole and/or trimethoprim, amoxicillin and/or clavulanate, and levofloxacin. In some variations the ascaroside may be compounded with the antibiotic for concurrent delivery.

The gut microbiome may be involved in the synthesis of vitamins, fine tuning of host immunity, fermentation of insoluble fibers, integrity of intestinal barrier, modification of bile acids, neurotransmitters and hormones. Diversity of the gut microbiome has been linked to health through a number of studies, demonstrating that decreased alpha or beta diversity are linked to a diseased state. Alpha diversity refers to the species diversity, whereas beta diversity is the ratio between regional and local species diversity.

The methods described herein may also be used to treat disorders that reduce gut microbiota diversity. For example, there is a correlation between ADHD and reduced alpha diversity. Thus, the methods described herein may be method of treating ADHD by increasing diversity (and in particular, alpha diversity) of the patient's gut microbiota. Also described herein are methods of treating anxiety and stress by increasing gut microbiota diversity. For example, sociability is associated with higher gut microbiota diversity, and anxiety and stress with reduced diversity.

Alpha diversity is reduced in fatigued cancer patients. Described herein are methods of treating the effects of cancer, or of improving cancer therapies, by increasing gut microbiota diversity.

Obesity and changes in microbiota composition are also correlated with reduced but microbiota bacterial diversity. In overweight/obese humans, low fecal bacterial diversity is associated with more marked overall adiposity and dyslipidemia, impaired glucose homeostasis and higher low-grade inflammation. Thus, described herein are methods of treating obesity by increasing gut microbiota diversity.

### Constipation

The methods and compositions described herein may also be used to treat constipation. Constipation, which accompanies various diseases, may be a symptom of underlying defects in either transit of fecal mass through the gut or defecation, a commonly diagnosed functional gastrointestinal disorder. It also associates with gastrointestinal motility disorders, which is characterized by a series of complex gastrointestinal symptoms in absence of mechanical obstruction of the gastrointestinal tract. Consistency of the stool is usually described using the Bristol Stool Chart which shows a spectrum of stools where Type 1 is more dry and hard and Type 7 is more loose and watery.

Surprising, treatment of subjects with an ascaroside resulted in an increase in the proportion of feces having a stool type 4 on the Bristol Stool Chart, which is considered optimal. For example, as shown in FIG. 9, the proportion of feces in mice treated with ascaroside (e.g., Ascr #7) increased significantly from type 3 (more constipated) to type 4 (more normal, described as “smooth and soft”). This data demonstrates that the proportion of feces in mice treated with Ascr #7 had a higher proportion of feces with Type 4 stools. Thus, these methods and compositions may be used to treat constipation in a subject.

Any appropriate dose of the compositions described herein may be used. For example, a dose appropriate for treating a human or non-human primate may be between about 0.1 mg and 10 mg (e.g., between about 1 mg and 10 mg, etc.). In some variations the dosage amount may be less, especially if it is a combination of different pheromones. For example the dosage delivered may be greater than 0.1 mg daily (e.g., 0.2 mg/daily, 0.3 mg/day, 0.4 mg/day, 0.5 mg/day, 1 mg/day etc.). In some compositions, e.g., in which the composition is configured to be taken orally and pass through the stomach to reach the gut microbiota, the dose may be even higher than 10 mg.

In some variations, the dose may be a single dose, two doses, three doses, four doses, five doses, etc. per patient per day. The dose may be delivered at once, or via a timed-release. The dose may be configured to release the ascaroside within the patient' gut. For example the dose may be pH released, in which the dose of ascaroside is released based on the surrounding pH. Note that the ascaroside pheromones described herein may change to other isomers at different pH, or the sugar ring may open at low pH, or the carboxylic acid may undergo a reaction at high pH. Also the ascaroside may hydrolyze in the presence of water, but not likely that much as they have been stored in water for years with high stability. As mentioned above, the ascaroside may be modified to enhance delivery, efficacy, half-life, etc.

As will be described herein below, a variety of dose forms may be used. As, in some variations, the ascaroside is intended to treat the microbiota of the patient's gut, the dose may be configured to effect the gut microbiome and not significantly affect the mammalian host. For example, oral doses may be preferred. Note that any microbiome may be treated as described herein, not limited to gut. For example, the vaginal microbiome may be treated as described.

The dose may be substantially smaller for oral doses to treat the microbiome as compared to doses configured to treat a patient disorder (see, e.g. U.S. Pat. No. 9,868,754, incorporated by reference herein). The methods and apparatuses described herein may use one or more ascaroside analogs, which may be modified to have a higher half-life, greater permeability, etc.

Another example of a pheromone that may be use is Ascr #6.1:

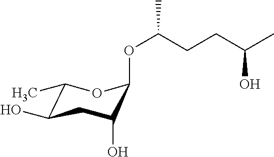

As mentioned above, any of the pheromones (ascarosides) described herein may be part of a composition that is configured for delayed release and/or slow-release, sustained release, etc. For example, a binding agents may be included in the compositions, to allow for sustained release or slow release.

Any of these compositions may include an excipient, diluent, or carrier. In some variations, the composition may include a cooling or heating additive, such as menthol. The compositions may contain a pharmaceutically acceptable excipient, diluent, or carrier in addition to the mixture.

Although oral compositions may be preferred to treating a microbiome (e.g., gut microbiome, vaginal microbiome, etc.), in some variations, a topical formulation may be used. In some variations, the excipient, diluent, or carrier may be configured for topical application. For example, the excipient, diluent, or carrier may comprise an emulsifying agent. In general, an excipient, diluent or carrier (including water) is an inactive substance that serves as the vehicle or medium for a drug or other active substance.

Excipients may include bulking agents, fillers or the like. The excipient may aid in the handling of the mixture of active substances by facilitating powder flowability or non-stick properties, aiding in vitro stability (e.g., prevention of denaturation or aggregation over the expected shelf life), enhancing solubility, improving absorption and/or uptake, providing better aesthetic and/or cosmetic features, altering physical properties etc.

Examples of excipients may include: anti-adherents (e.g., magnesium stearate, etc.); binders (e.g., saccharides and their derivatives: disaccharides, sucrose, lactose; polysaccharides and their derivatives: starches, cellulose or modified cellulose such as microcrystalline cellulose and cellulose derivatives including cellulose ethers such as hydroxypropyl cellulose; sugar alcohols such as xylitol, sorbitol or mannitol; protein: gelatin; synthetic polymers: polyvinylpyrrolidone or PVP, polyethylene glycol or PEG, polyvinylpyrrolidone, starch, sucrose and polyethylene glycol, methyl cellulose); coatings (e.g., cellulose ether hydroxypropyl methylcellulose, synthetic polymers, shellac, corn protein zein or other polysaccharides, gelatin); enterics (fatty acids, waxes, shellac, plastics, and plant fibers); colors (titanium oxide, azo dyes, etc.); disintegrants (e.g., crosslinked polymers: crosslinked polyvinylpyrrolidone such as crospovidone, crosslinked sodium carboxymethyl cellulose or croscarmellose sodium, glycolate, etc.); flavors (fruit extract, etc.); glidants (e.g., fumed silica, talc, and magnesium carbonate, etc.); lubricants (e.g., talc or silica, and fats, e.g. vegetable stearin, magnesium stearate or stearic acid, etc.); preservatives (e.g., antioxidants like vitamin A, vitamin E, vitamin C, retinyl palmitate, and selenium; cysteine, methionine; citric acid, sodium citrate; parabens: methyl paraben and propyl paraben); sorbents; sweeteners (e.g., sugar); vehicles (petrolatum, dimethyl sulfoxide, mineral oil, etc.); emollient/stiffening agents (Carnauba wax, Cetyl alcohol, Cetyl ester wax, Emulsifying wax, Hydrous lanolin, Lanolin, Lanolin alcohols, Microcrystalline wax, Paraffin, Petrolatum, Polyethylene glycol, Stearic acid, Stearyl alcohol, White wax, Yellow wax, etc.); emulsifier/emulsifying agent/solubilizing agent (Polysorbate 20, Polysorbate 80, Polysorbate 60, Poloxamer, Emulsifying wax, Sorbitan monostearate, Sorbitan monooleate, Sodium lauryl sulfate, Propylene glycol monostearate, Diethylene glycol monoethyl ether, Docusate sodium, etc.); humectant (e.g., Glycerin, Propylene glycol, Polyethylene glycol, Sorbitol solution, 1,2,6 Hexanetriol, etc.); thickening/gelling agent (Carbomer, Methyl cellulose, Sodium carboxyl methyl cellulose, Carrageenan, Colloidal silicon dioxide, Guar gum, Hydroxypropyl cellulose, Hydroxypropyl methyl cellulose, Gelatin, Polyethylene oxide, Alginic acid, Sodium alginate, Fumed silica, etc.); preservative (Benzoic acid, Propyl paraben, Methyl paraben, Imidurea, Sorbic acid, Potassium sorbate, Benzalkonium chloride, Phenyl mercuric acetate, Chlorobutanol, Phenoxyethanol, etc.); permeation enhancer (Propylene glycol, Ethanol, Isopropyl Alcohol, Oleic acid, Polyethylene glycol, etc.); chelating agent (Ethylene diamine tetraacetate, etc.); acidifying/alkalizing/buffering agent (Citric acid, Phosphoric acid, Sodium hydroxide, Monobasic sodium Phosphate, Trolamine, etc.); vehicle/solvent (Purified water, Hexylene glycol, Propylene glycol, Oleyl alcohol, Propylene carbonate, Mineral oil, etc.). These examples may be redundant, and different excipients may be used for different reasons, and may have dual or multiple functionalities.

The composition may be configured as a liquid or emulsion in a form suitable for ingesting and/or topical administration to a human, including a spray, lotion, cream, ointment, tincture, etc.

Also described herein are methods of treating a subject to modify (e.g., improve, restore, rebalance, etc.) the patient's microbiome. The method may include using a composition including one or more ascaroside to modify the population(s) of bacteria within the patient's microbiome. For example, any of these methods may be configured to increase one or more type of bacterium (e.g., Bifidobacterium) abundance in the subject and/or reduces levels of one or more other type of bacterium (e.g., E. coli and Shigella). Thus in some variations the method may result in an increase in the relative ratio of butyrate, particularly in the elderly, and may significantly reduce the use of stool softeners.

The general composition of the gut microbiome has been reported to change with aging whereby there is in some cases a decrease in Actinobacteria and Bacteroidetes and an increase in Firmicutes. In some variations, the methods described herein may reverse all or some of these effects. For example, these methods may increase Actinobacteria and/or Bacteroidetes, and/or may decrease Firmicutes.

The gut microbiome in humans is a complex ecosystem that works in harmony with the human gastrointestinal tract providing key metabolic end-products that are essential to the health of the host.

In some variations, the methods described herein may be used to treat one or more indication related to (caused by or increased by) a problem with the gut microbiome. For example, dysbiosis of the gut microbiome has been associated with illnesses such as irritable bowel syndrome, and Clostridium difficile disease.

In some variations, the methods described herein may increase the diversity of bacteria in the microbiome compared to control.

The naturally-occurring Ascarosides (ascrs #1, #2, #3, #5, #7, #9, #10, #11, #12, #14, #16, #18, #20, #22, #24, and #26) selectively alter the gut microbiota of experimental model animals. Although the degree of modification may vary (e.g., ascr #7 vs. ascr #9 in FIG. 3 vs. FIG. 4), preliminary experiments suggest that these pheromones may, as a class, alter the gut microbiota, and in particular may increase diversity of the gut microbiota.

Combinations of any of the naturally-occurring Ascarosides may also be used in any of these methods or compositions for performing these methods. For example, combination of any two or more of ascrs #1, #2, #3, #5, #7, #9, #10, #11, #12, #14, #16, #18, #20, #22, #24, and #26 (e.g., any three or more of ascrs #1, #2, #3, #5, #7, #9, #10, #11, #12, #14, #16, #18, #20, #22, #24, and #26, and four or more of ascrs #1, #2, #3, #5, #7, #9, #10, #11, #12, #14, #16, #18, #20, #22, #24, and #26, any five or more of ascrs #1, #2, #3, #5, #7, #9, #10, #11, #12, #14, #16, #18, #20, #22, #24, and #26, etc.), may be used in the methods described herein, and/or may be formulated into a therapeutic composition as described herein.

In addition, ascarosides having the formula of formula II (which overlap with the naturally-occurring ascarosides) may be used in any of these methods or compositions for performing these methods. Any combination of these ascarosides may be used, including an combination with one or more of the naturally-occurring ascarosides (e.g., ascrs #1, #2, #3, #5, #7, #9, #10, #11, #12, #14, #16, #18, #20, #22, #24, and #26).

Preliminary data also suggests that the Ascaroside derivatives, including those described as any of compounds 1-56, in table 3 above may be used successfully in any of these methods or compositions for performing these methods. For example, a composition may include an Ascaroside derivatives having the formula of Formula (III), as described above (including, but not limited to those described in table 3, above). For example, a composition for increasing gut microbiota diversity may include one or more compounds having a formula of Formula III. In some variations, the composition may include any of the ascaroside derivative compounds, 1-56 of table 3, above. A composition for treating constipation may include one or more compounds having a formula of Formula III. In some variations, the composition may include any of the ascaroside derivative compounds, 1-56 of table 3, above.

Also described herein are compounding including mixtures of and any of the ascaroside derivatives (e.g., any of the ascaroside derivative compounds, 1-56 of table 3, above) and one or more naturally-occurring ascaroside.

For example, table 4, below, lists ascarosides having the formula of formula I, including naturally-occurring ascarosides (some of which ascarosides having the formula of formula II), and ascaroside derivatives (e.g., ascarosides having the formula of formula III). As mentioned above, the compositions described herein may be combined as combination of any of the listed ascarosides, such as, but not limited to: ascr #1 and any one or more of: ascr #2, ascr #3, ascr #5, ascr #7, ascr #9, ascr #10, ascr #11, ascr #12, ascr #14, ascr #16, ascr #18, ascr #20, ascr #22, ascr #24, ascr #26, ascr #15, ascr #17, ascr #19, ascr #21, ascr #23, ascr #25, ascr #9.2, mbas #3, oscr #10, ascr #4, bhas #18, hbas #3, easc #18, oscr #9, ascr #8, icas #9, ascr #6.1, ascr #6.2, bhas #24, bhas #26, bhas #28, bhas #30, Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, and Compound 56; ascr #7 and any one or more of: ascr #1, ascr #2, ascr #3, ascr #5, ascr #9, ascr #10, ascr #11, ascr #12, ascr #14, ascr #16, ascr #18, ascr #20, ascr #22, ascr #24, ascr #26, ascr #15, ascr #17, ascr #19, ascr #21, ascr #23, ascr #25, ascr #9.2, mbas #3, oscr #10, ascr #4, bhas #18, hbas #3, easc #18, oscr #9, ascr #8, icas #9, ascr #6.1, ascr #6.2, bhas #24, bhas #26, bhas #28, bhas #30, Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, and Compound 56; ascr #9 and any one or more of: ascr #1, ascr #2, ascr #3, ascr #5, ascr #7, ascr #10, ascr #11, ascr #12, ascr #14, ascr #16, ascr #18, ascr #20, ascr #22, ascr #24, ascr #26, ascr #15, ascr #17, ascr #19, ascr #21, ascr #23, ascr #25, ascr #9.2, mbas #3, oscr #10, ascr #4, bhas #18, hbas #3, easc #18, oscr #9, ascr #8, icas #9, ascr #6.1, ascr #6.2, bhas #24, bhas #26, bhas #28, bhas #30, Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, and Compound 56; Compound 1 and any one or more of: ascr #1, ascr #2, ascr #3, ascr #5, ascr #7, ascr #9, ascr #10, ascr #11, ascr #12, ascr #14, ascr #16, ascr #18, ascr #20, ascr #22, ascr #24, ascr #26, ascr #15, ascr #17, ascr #19, ascr #21, ascr #23, ascr #25, ascr #9.2, mbas #3, oscr #10, ascr #4, bhas #18, hbas #3, easc #18, oscr #9, ascr #8, icas #9, ascr #6.1, ascr #6.2, bhas #24, bhas #26, bhas #28, bhas #30, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, a Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, and Compound 56, etc.

TABLE FOUR

| 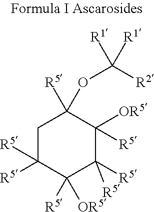 | Naturally occurring Ascarosides | 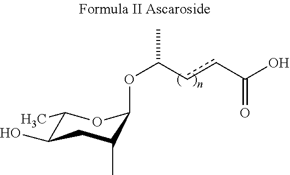 | 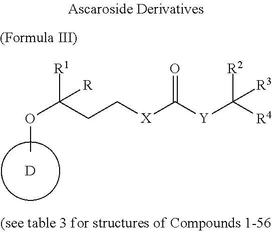 |
|---|---|---|---|
| | ascr#5 | | |
| | ascr#1 | | Compound 1 |
| | ascr#2 | | Compound 2 |
| | ascr#3 | | Compound 3 |
| | ascr#7 | | Compound 4 |
| | ascr#9 | | Compound 5 |
| | ascr#10 | | Compound 6 |
| | ascr#l l | | Compound 7 |
| | ascr#12 | | Compound 8 |
| | ascr#14 | | Compound 9 |
| | ascr#16 | | Compound 10 |
| | ascr#18 | | Compound 11 |
| | ascr#20 | | Compound 12 |
| | ascr#22 | | Compound 13 |
| | ascr#24 | | Compound 14 |
| | ascr#26 | | Compound 15 |
| | | | Compound 16 |
| | | | Compound 17 |
| | | | Compound 18 |
| | | | Compound 19 |
| | | | Compound 20 |
| | | | Compound 21 |
| | | | Compound 22 |
| | | | Compound 23 |
| | | | Compound 24 |
| | | | Compound 25 |
| | | | Compound 26 |
| | | | Compound 27 |
| | | | Compound 28 |
| | | | Compound 29 |
| | | | Compound 30 |
| | | | Compound 31 |
| | | | Compound 32 |
| | | | Compound 33 |
| | | | Compound 34 |
| | | | Compound 35 |
| | | | Compound 36 |
| | | | Compound 37 |
| | | | Compound 38 |
| | | | Compound 39 |
| | | | Compound 40 |
| | | | Compound 41 |
| | | | Compound 42 |
| | | | Compound 43 |
| | | | Compound 44 |
| | | | Compound 45 |
| | | | Compound 46 |
| | | | Compound 47 |
| | | | Compound 48 |
| | | | Compound 49 |
| | | | Compound 50 |
| | | | Compound 51 |
| | | | Compound 52 |
| | | | Compound 53 |
| | | | Compound 54 |
| | | | Compound 55 |
| | | | Compound 56 |
| ascr#15 | | | |
| ascr#17 | | | |
| ascr#19 | | | |
| ascr#21 | | | |
| ascr#23 | | | |
| ascr#25 | | | |
| ascr#9.2 | | | |
| mbas#3 | | | |
| oscr#10 | | | |
| ascr#4 | | | |
| bhas#18 | | | |
| hbas#3 | | | |
| ease# 18 | | | |
| oscr#9 | | | |
| ascr#8 | | | |
| icas#9 | | | |
| ascr#6.1 | | | |
| ascr#6.2 | | | |
| bhas#24 | | | |
| bhas#26 | | | |
| bhas#28 | | | |
| bhas#30 | | | |

As described above, any of the ascarosides in table 4, individually or in combination may be used to increase gut microbiota diversity (e.g., alpha diversity), and/or may be used to increase one or more of the amount of: Bifidobacterium, Akkermansia and/or Adlercreutzia. Any of the ascarosides in table 4, individually or in combination may be included as part of a composition to increase gut microbiota diversity. Any of the ascarosides in table 4, individually or in combination may be used to treat constipation, or may be part of a composition to treat constipation.

Any of the compositions and/or methods described herein (e.g., including any of the ascarosides in table 4, individually or in combination) may be used to treat the skin microbiome. For example, a composition of one or more ascaroside may be topically applied. Any of these compositions and/or methods may be used to treat nasal (or respiratory) microbiome. For example, any of the compositions described herein may be formulated as an inhalational composition.

For example, a method as described herein may generally include: administering to said patient a therapeutically effective amount of a composition including one or more ascaroside (e.g., including any of the ascarosides in table 4, individually or in combination). Administering may comprise applying the composition to the patient's body cavity (e.g., gut, vagina, etc.) or skin microbiome. For example, administering may comprise spraying the composition, orally delivering the composition, etc. Alternatively or additionally, administering may comprise applying the composition systemically to the patient. The compositions described herein may also be used as a coating (e.g., to a medical device, implant, etc.).

As mentioned, the therapeutic agents (compositions) described herein may include an excipient, diluent, or carrier.

The compositions described herein may be used to directly treat a patient (e.g., human or non-human animals), as a coating for a medical device or implant, or in any other use in which a microbiome adjustment would be useful.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise. As used herein, the terms below have the meanings indicated.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for selectively altering a subject's gut microbiota, the method comprising: administering to the subject a composition comprising pharmaceutically-acceptable carrier and an effective amount of ascaroside thereby selectively modifying the composition of the gut microbiota.

2. The method of claim 1, wherein administering comprises orally administering.

3. The method of claim 1, wherein administering comprises administering into the subject's gastrointestinal tract.

4. The method of claim 1, wherein administering comprises one or more of: oral, intravenous, intraperitoneal and intramuscular administration.

5. The method of claim 1, wherein administering comprises administering a suppository comprising ascaroside.

6. The method of any of claims 1-5, wherein administering comprises administering a time-release formulation of ascaroside.

7. The method of any of claims 1-5, wherein administering comprises administering multiple doses of ascaroside daily.

8. The method of any of claims 1-7, wherein modifying the composition of the gut microbiota comprises increase the amount of one or more of Bifidobacterium, Akkermansia and Adlercreutzia.

9. The method of any of claims 1-7, wherein modifying the composition of the gut microbiota comprises increase the amount of Bifidobacter catenulatum.

10. The method of any of claims 1-9, wherein modifying the composition of the gut microbiota comprises increase the diversity of the gut microbiota.

11. The method of any of claims 1-7, wherein modifying the composition of the gut microbiota comprises increasing the diversity of the gut microbiota and increasing the amount of one or more of: Bifidobacterium, Akkermansia and Adlercreutzia.

12. The method of any of claims 1-11, wherein administering comprises administering to the subject during or immediately after a course of antibiotics.

13. The method of any of claims 1-12, wherein the ascaroside comprises a glycolipid containing an ascarylose.

14. The method of any of claims 1-13, wherein the ascaroside comprises a naturally-occurring ascaroside comprising one or more of: ascr #1, ascr #2, ascr #3, ascr #5, ascr #7, ascr #9, ascr #10, ascr #11, ascr #12, ascr #14, ascr #16, ascr #18, ascr #20, ascr #22, ascr #24, ascr #26.

15. The method of any of claims 1-13, wherein the ascaroside comprises a formula of Formula II: 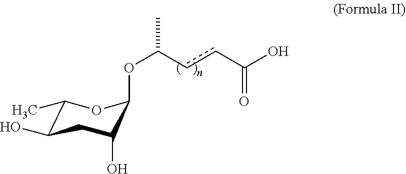
wherein [symbol] represents a double or single bond, and wherein ( )ₙ represents (CH2)ₙ where n is between 1-12, or salt thereof.

16. The method of any of claims 1-15, wherein the ascaroside comprises a plurality of different ascarosides, or salts thereof.

17. The method of any of claims 1-15, wherein the ascaroside comprises a plurality of different ascarosides including one or more ascarosides having a formula of Formula II: 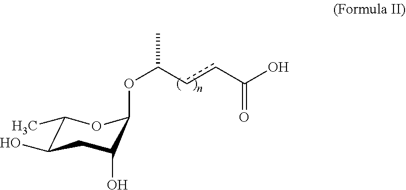
wherein [symbol] represents a double or single bond, and wherein ( )ₙ represents (CH2)ₙ where n is between 1-12, or salt thereof.

18. The method of any of claims 1-17, wherein the ascaroside comprises Ascr #7.

19. The method of any of claims 1-17, wherein the ascaroside comprises one or more of: Ascr #7, and Ascr #9.

20. The method of any of claims 1-13, wherein the ascaroside is an ascaroside derivative having a structure of Formula III: 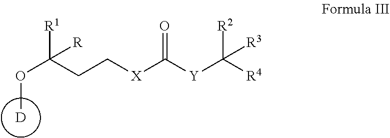
wherein
Ring D is selected from: 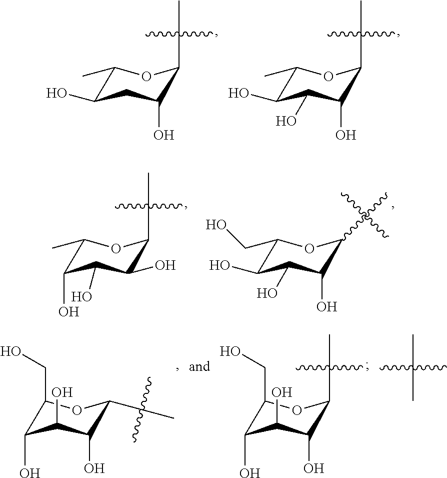
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

21. The method of claim 20, wherein X is —C(R⁵)₂—C(R⁵)₂—.

22. The method of claim 20, wherein X is E or Z —CH═CH—.

23. The method of any of claims 20-23, wherein Y is NR⁶.

24. The method of any of claims 20-23, wherein Y is O.

25. The method of any of claims 20-24, wherein
R² is H;
R³ is H or CH₃; and
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl, or, when Y is NR⁶, R⁴ and NR⁶, taken together with the carbon atom to which they are bonded, form a 5- or 6-membered heterocyclic ring.

26. The method of any of claims 20-25, wherein R², R³ and R⁴ are each CH₃.

27. The method of any of claims 20-26, wherein the compound is selected from

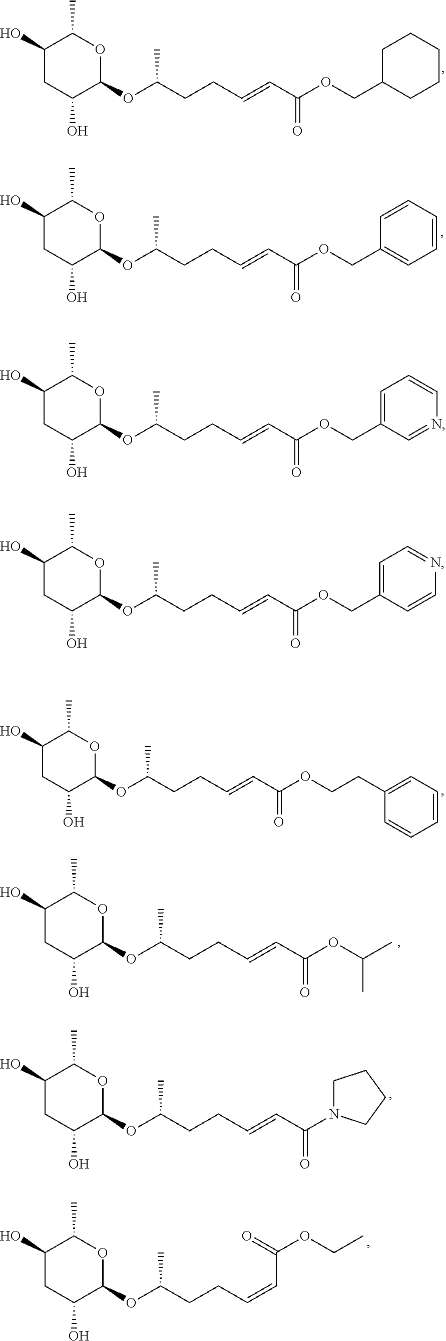

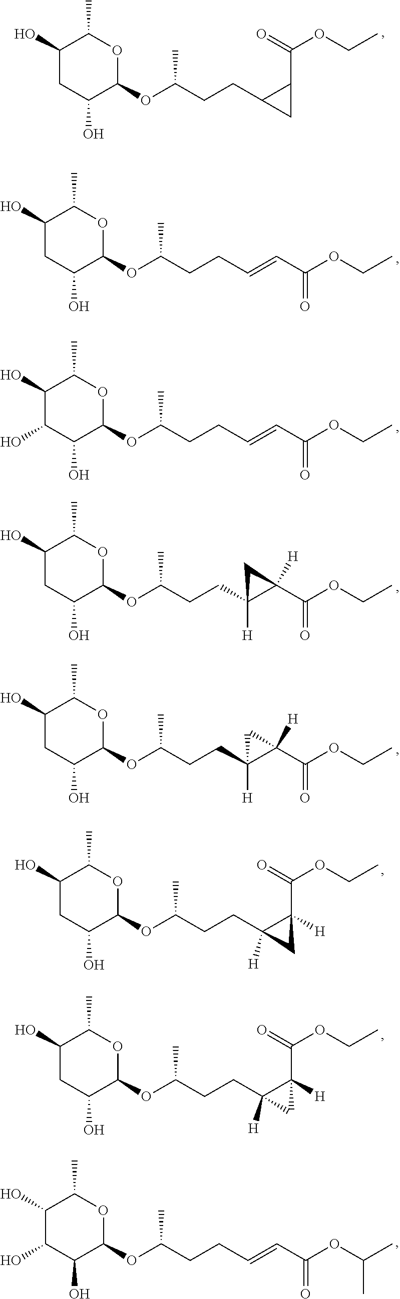

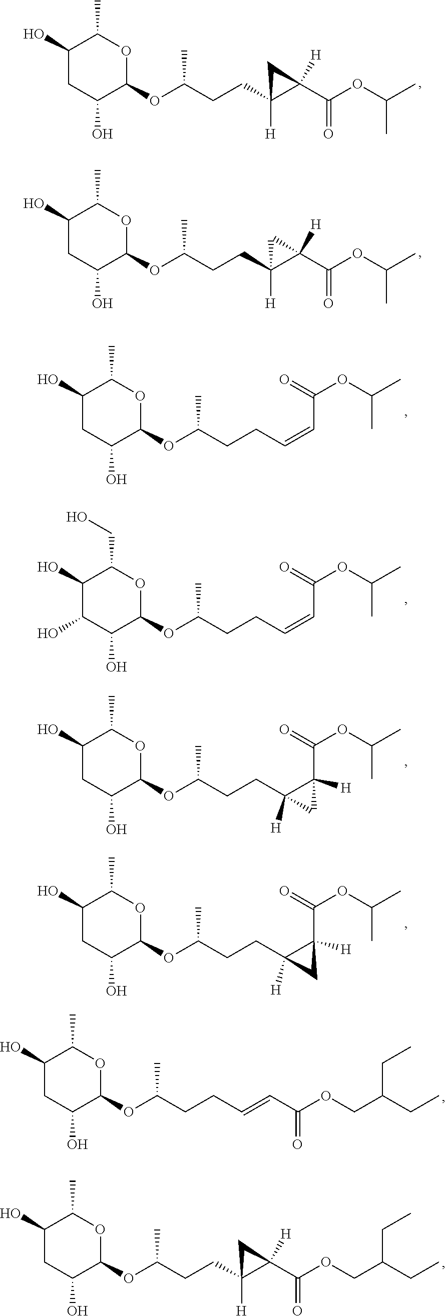

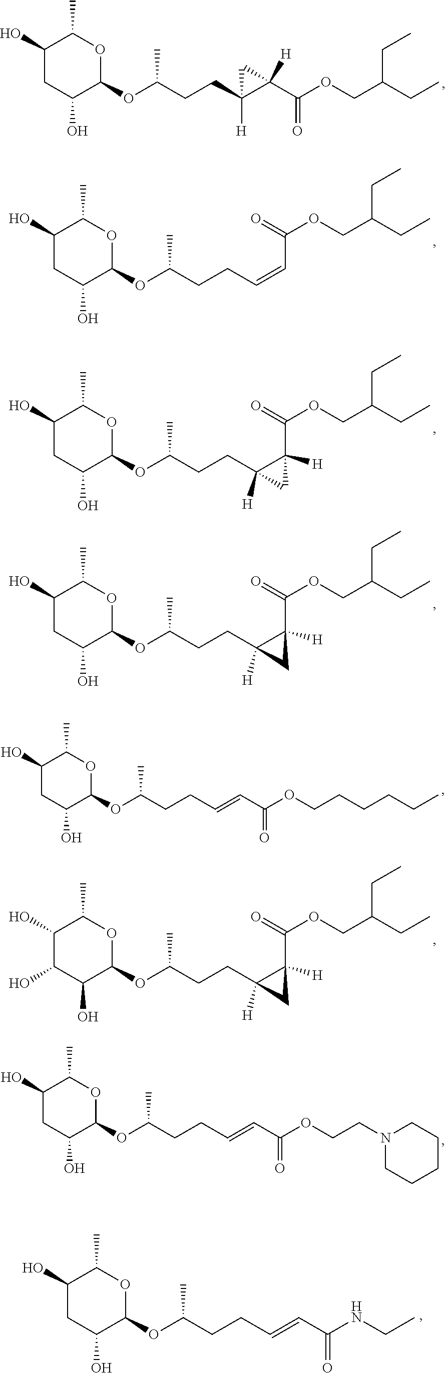

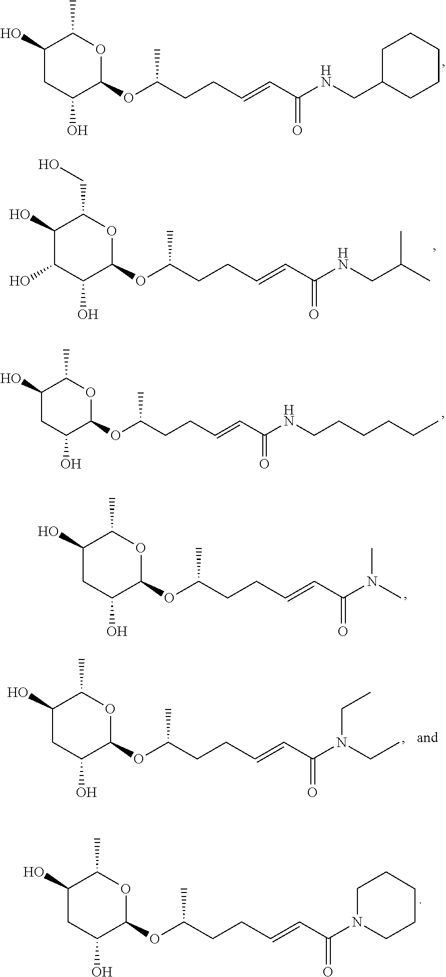

28. The method of claim 26, wherein the compound is selected from

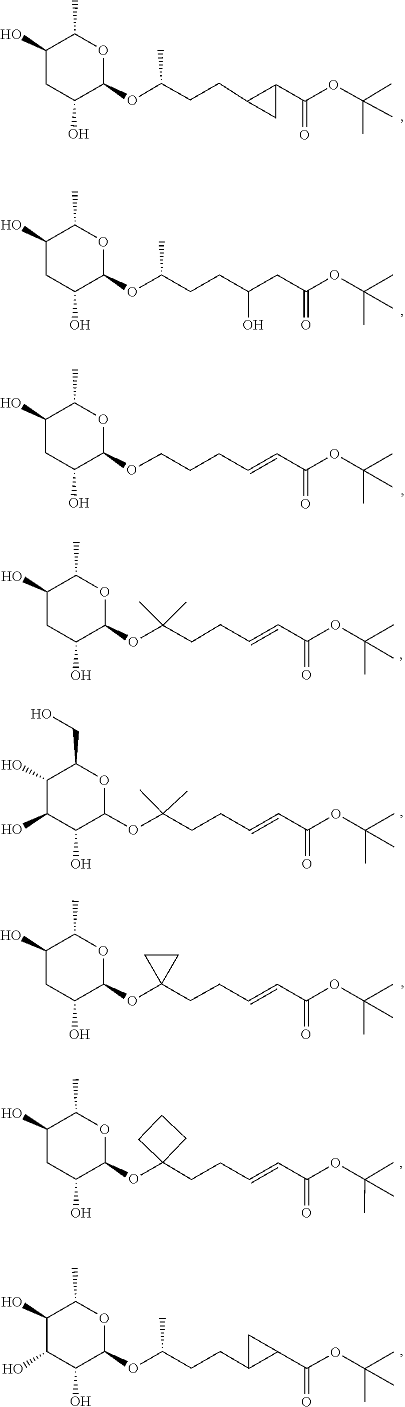

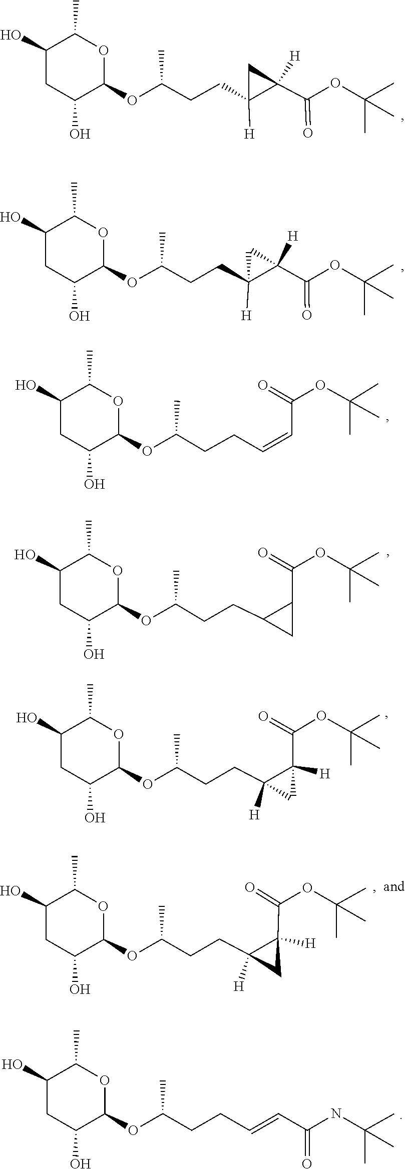

29. The method of claim 20, wherein the compound has the structure of Formula IV: 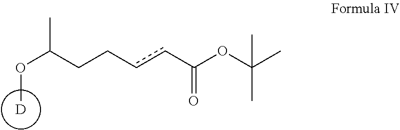
wherein
Ring D is selected from: 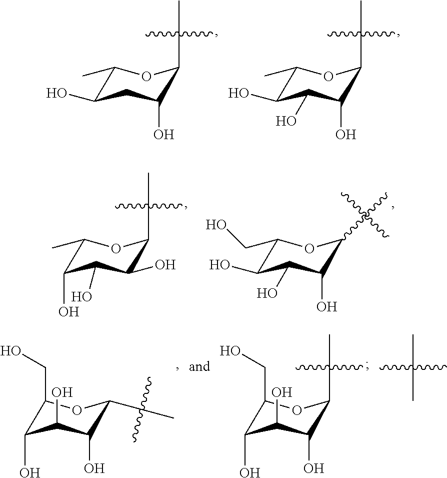
represents the point of attachment of Ring D to the oxygen atom; and [symbol]
is a double bond or a single bond.

30. The method of claim 29, wherein the compound has the structure:

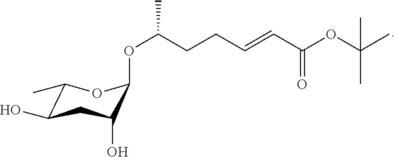

31. The method of claim 29, wherein the compound has the structure:

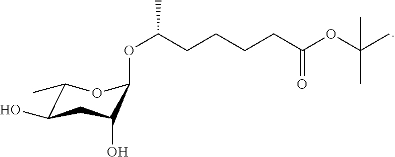

32. The method of claim 29, wherein the compound is selected from

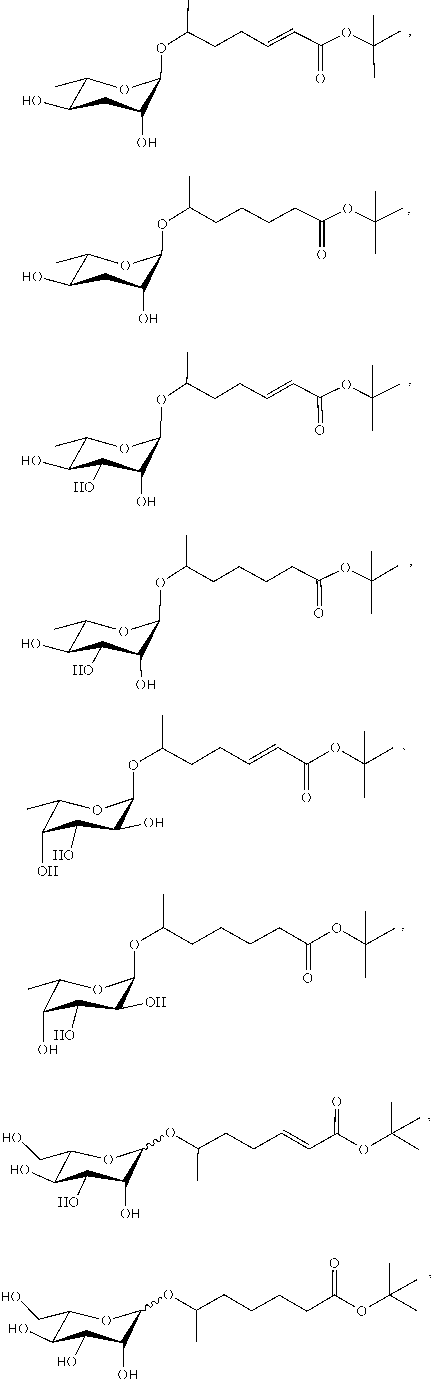

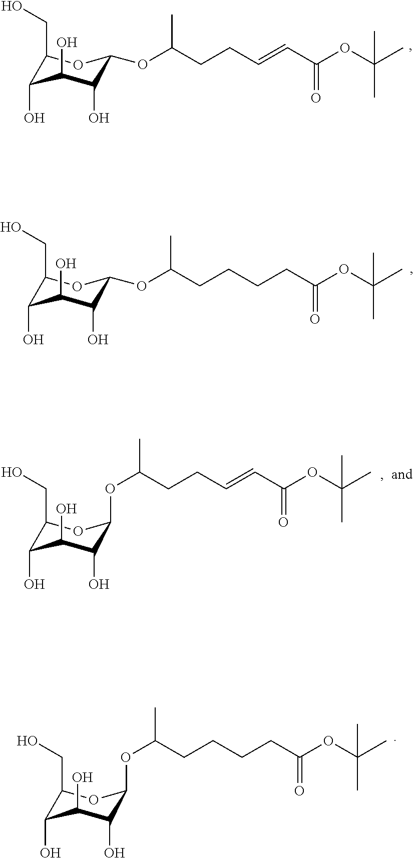

33. The method of any of claims 1-32, wherein the ascaroside comprises between 0.1% and 99% of the composition.

34. A method for selectively altering a subject's gut microbiota, the method comprising: administering to the subject's gastrointestinal system a composition comprising pharmaceutically-acceptable carrier and an effective amount of ascaroside thereby selectively modifying the composition of the gut microbiota to increase one or more of: the diversity of the gut microbiota, the amount of Bifidobacterium, the amount of Akkermansia and the amount of Adlercreutzia within the subject's gastrointestinal system.

35. A method of increasing the diversity of a subject's gut microbiota, the method comprising: administering to the subject a therapeutically effective amount of a composition of ascaroside so that the ascaroside modifies the diversity of the subject's gut microbiota within the subject's intestinal tract.

36. The method of claim 35, wherein administering comprises orally administering.

37. The method of claim 35, wherein administering comprises administering into the subject's gastrointestinal tract.

38. The method of claim 35, wherein administering comprises one or more of: oral, intravenous, intraperitoneal and intramuscular administration.

39. The method of claim 35, wherein administering comprises administering a suppository comprising ascaroside.

40. The method of claim 35, wherein administering comprises administering a time-release formulation of ascaroside.

41. The method of claim 35, wherein administering comprises administering multiple doses of ascaroside daily.

42. The method of claim 35, wherein administering comprises administering to the subject during or immediately after a course of antibiotics.

43. The method of claim 35, wherein the ascaroside comprises a glycolipid containing an ascarylose.

44. The method of claim 35, wherein the ascaroside comprises a formula of Formula II: 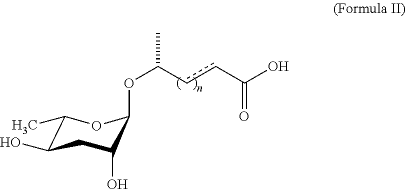
wherein [symbol] represents a double or single bond, and wherein ( )ₙ represents (CH2)ₙ where n is between 1-12, or salt thereof.

45. The method of claim 44, wherein the ascaroside comprises a plurality of different ascarosides, or salts thereof.

46. The method of claim 44, wherein the ascaroside comprises a plurality of different ascarosides including one or more ascarosides having a formula of Formula II: 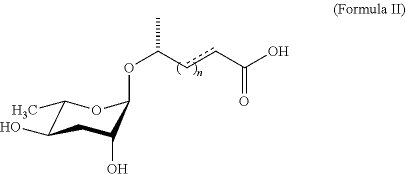
wherein [symbol] represents a double or single bond, and wherein ( )ₙ represents (CH2)ₙ where n is between 1-12, or salt thereof.

47. The method of claim 44, wherein the ascaroside comprises Ascr #7.

48. The method of claim 44, wherein the ascaroside comprises one or more of: Ascr #7, and Ascr #9.

49. The method of claim 35, wherein the ascaroside is an ascaroside derivative having a structure of Formula III: 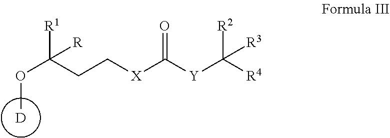
wherein
Ring D is selected from: 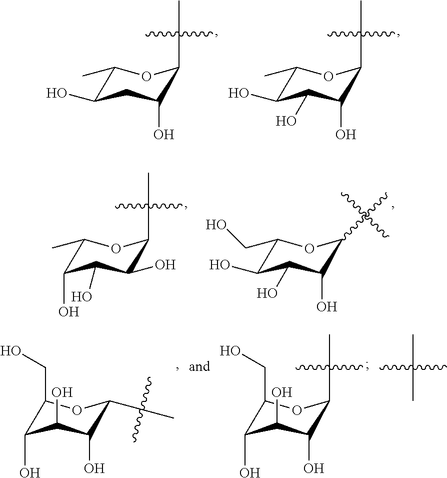
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

50. The method of claim 44, wherein the ascaroside comprises between 0.1% and 99% of the composition.

51. A method of increasing the diversity of a subject's gut microbiota, the method comprising: administering to the subject's gastrointestinal system a composition comprising pharmaceutically-acceptable carrier and an effective amount of ascaroside so that within the subject's intestinal tract, the ascaroside modifies the diversity of the subject's gut microbiota.

52. A method of treating constipation in a subject, the method comprising administering to the subject a composition of ascaroside to increase fecal softness.

53. The method of claim 52, wherein administering comprises orally administering.

54. The method of claim 52, wherein administering comprises administering into the subject's gastrointestinal tract.

55. The method of claim 52, wherein administering comprises one or more of: oral, intravenous, intraperitoneal and intramuscular administration.

56. The method of claim 52, wherein administering comprises administering a suppository comprising ascaroside.

57. The method of claim 52, wherein administering comprises administering a time-release formulation of ascaroside.

58. The method of claim 52, wherein administering comprises administering multiple doses of ascaroside daily.

59. The method of claim 52, wherein increasing fecal softness comprises increase the amount of one or more of Bifidobacterium, Akkermansia and Adlercreutzia.

60. The method of claim 52, wherein increasing fecal softness comprises increase the amount of Bifidobacter catenulatum.

61. The method of claim 52, wherein increasing fecal softness comprises increase the diversity of the gut microbiota.

62. The method of claim 52, wherein increasing fecal softness comprises increasing the diversity of the gut microbiota and increasing the amount of one or more of: Bifidobacterium, Akkermansia and Adlercreutzia.

63. The method of claim 52, wherein the ascaroside comprises a glycolipid containing an ascarylose.

64. The method of claim 52, wherein the ascaroside comprises a formula of Formula II: 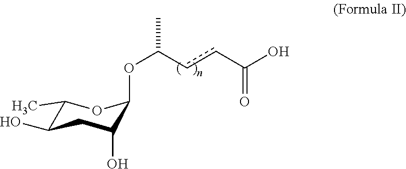
wherein [symbol] represents a double or single bond, and wherein ( )ₙ represents (CH2)ₙ where n is between 1-12, or salt thereof.

65. The method of claim 64, wherein the ascaroside comprises a plurality of different ascarosides, or salts thereof.

66. The method of claim 64, wherein the ascaroside comprises a plurality of different ascarosides including one or more ascarosides having a formula of Formula II: 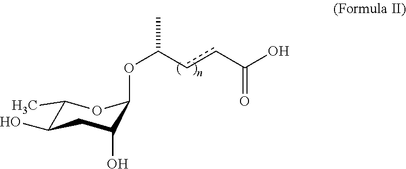
wherein [symbol] represents a double or single bond, and wherein ( )ₙ represents (CH2)ₙ where n is between 1-12, or salt thereof.

67. The method of claim 64, wherein the ascaroside comprises Ascr #7.

68. The method of claim 64, wherein the ascaroside comprises one or more of: Ascr #7, and Ascr #9.

69. The method of claim 52, wherein the ascaroside is an ascaroside derivative having a structure of Formula III: 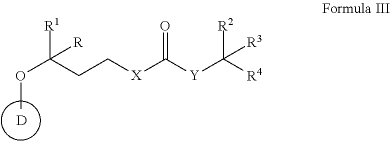
wherein
Ring D is selected from: 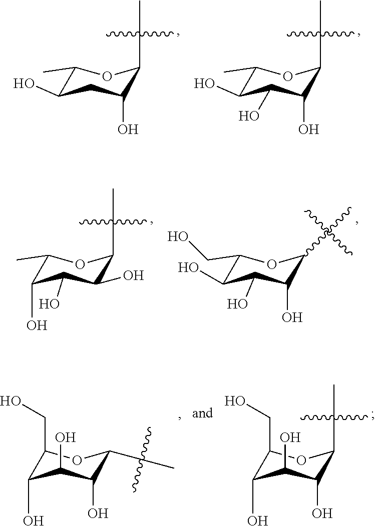
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

70. The method of claim 52, wherein the ascaroside comprises between 0.1% and 99% of the composition.

71. A method of treating constipation in a subject, the method comprising administering to the subject's gastrointestinal system a composition comprising pharmaceutically-acceptable carrier and an effective amount of ascaroside to increase fecal softness.

72. A composition of ascaroside for increasing the diversity of a subject's gut microbiota.

73. The composition of claim 72, wherein the ascaroside comprises a glycolipid containing an ascarylose.

74. The composition of claim 72, wherein the ascaroside comprises a formula of Formula II: 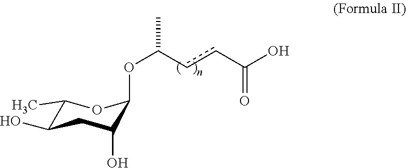
wherein [symbol] represents a double or single bond, and wherein ( )ₙ represents (CH2)ₙ where n is between 1-12, or salt thereof.

75. The composition of claim 72, wherein the ascaroside comprises a plurality of different ascarosides, or salts thereof.

76. The composition of claim 72, wherein the ascaroside comprises a plurality of different ascarosides including one or more ascarosides having a formula of Formula II: 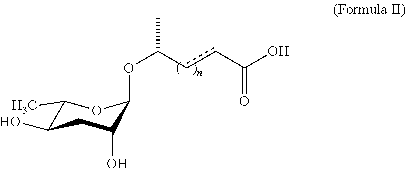
wherein [symbol] represents a double or single bond, and wherein ( )ₙ represents (CH2)ₙ where n is between 1-12, or salt thereof.

77. The composition of claim 76, wherein the ascaroside comprises Ascr #7.

78. The composition of claim 76, wherein the ascaroside comprises one or more of: Ascr #7, and Ascr #9.

79. The composition of claim 72, wherein the ascaroside is an ascaroside derivative having a structure of Formula III: 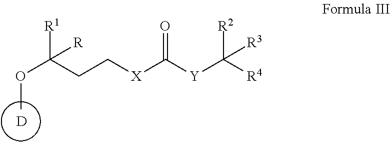
wherein
Ring D is selected from: 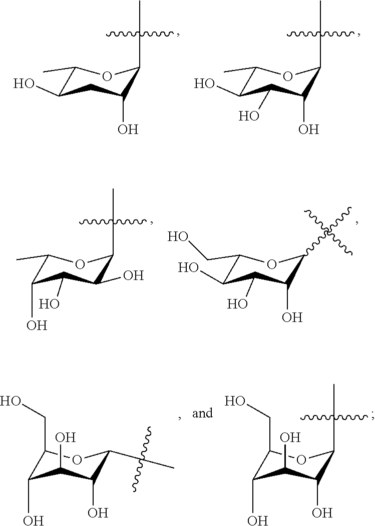
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

80. The composition of claim 72, wherein the ascaroside comprises between 0.1% and 99% of the composition.

81. The composition of claim 72, wherein further comprising a pharmaceutically acceptable carrier.

82. The composition of claim 72, further configured to release the ascaroside into an intestinal lumen over time.

83. The composition of claim 72, further configured as a time-release formulation.

84. The composition of claim 72, further configured for oral delivery.

85. The composition of claim 72, further configured as a suppository.

86. A composition of ascaroside for increasing the diversity of a subject's gut microbiota, the composition comprising between 1% and 99% of ascaroside by weight.

87. A composition of ascaroside for treating constipation in a subject.

88. The composition of claim 87, wherein the ascaroside comprises a glycolipid containing an ascarylose.

89. The composition of claim 87, wherein the ascaroside comprises a formula of Formula II: 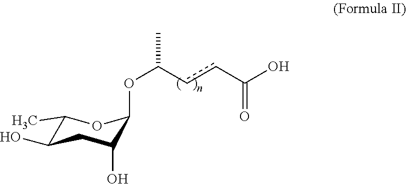
wherein [symbol] represents a double or single bond, and wherein ( )ₙ represents (CH2)ₙ where n is between 1-12, or salt thereof.

90. The composition of claim 87, wherein the ascaroside comprises a plurality of different ascarosides, or salts thereof.

91. The composition of claim 87, wherein the ascaroside comprises a plurality of different ascarosides including one or more ascarosides having a formula of Formula II: 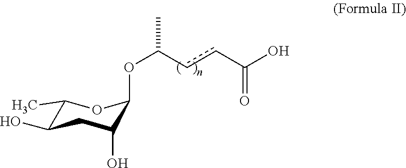
wherein [symbol] represents a double or single bond, and wherein ( )ₙ represents (CH2)ₙ where n is between 1-12, or salt thereof.

92. The composition of claim 91, wherein the ascaroside comprises Ascr #7.

93. The composition of claim 91, wherein the ascaroside comprises one or more of: Ascr #7, and Ascr #9.

94. The composition of claim 87, wherein the ascaroside is an ascaroside derivative having a structure of Formula III: 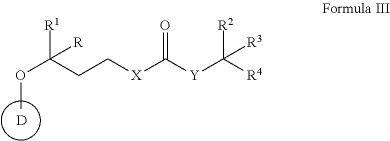
wherein
Ring D is selected from: 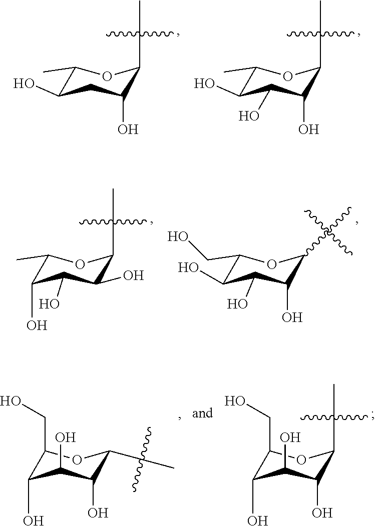
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

95. The composition of claim 87, wherein the ascaroside comprises between 0.1% and 99% of the composition.

96. The composition of claim 87, wherein further comprising a pharmaceutically acceptable carrier.

97. The composition of claim 87, further configured to release the ascaroside into an intestinal lumen over time.

98. The composition of claim 87, further configured as a time-release formulation.

99. The composition of claim 87, further configured for oral delivery.

100. The composition of claim 87, further configured as a suppository.

101. A composition of ascaroside for treating constipation in a subject, the composition comprising between 1% and 99% of ascaroside by weight.

102. A method for selectively altering a subject's gut microbiota, the method comprising: administering to the subject a composition comprising pharmaceutically-acceptable carrier and an effective amount of ascaroside derivative thereby selectively modifying the composition of the gut microbiota, wherein the ascaroside derivative has the structure of Formula III: 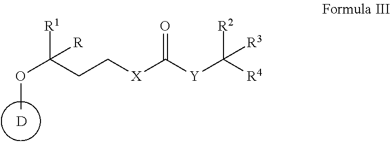
wherein
Ring D is selected from: 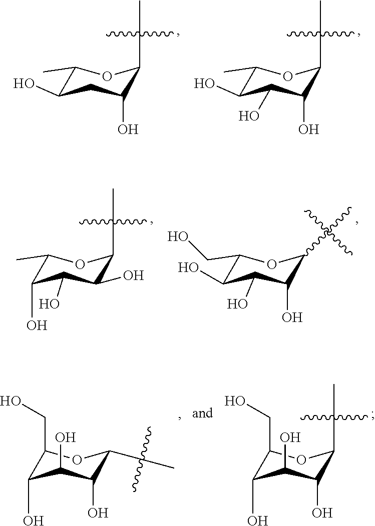
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

103. The method of claim 102, wherein X is —C(R⁵)₂—C(R⁵)₂—.

104. The method of claim 102, wherein X is E or Z —CH═CH—.

105. The method of any of claims 102-104, wherein Y is NR⁶.

106. The method of any of claims 102-104, wherein Y is O.

107. The method of any of claims 102-106, wherein
R² is H;
R³ is H or CH₃; and
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl, or, when Y is NR⁶, R⁴ and NR⁶, taken together with the carbon atom to which they are bonded, form a 5- or 6-membered heterocyclic ring.

108. The method of any of claims 102-106, wherein
R², R³ and R⁴ are each CH₃.

109. The method of any of claims 102-108, wherein the compound is selected from

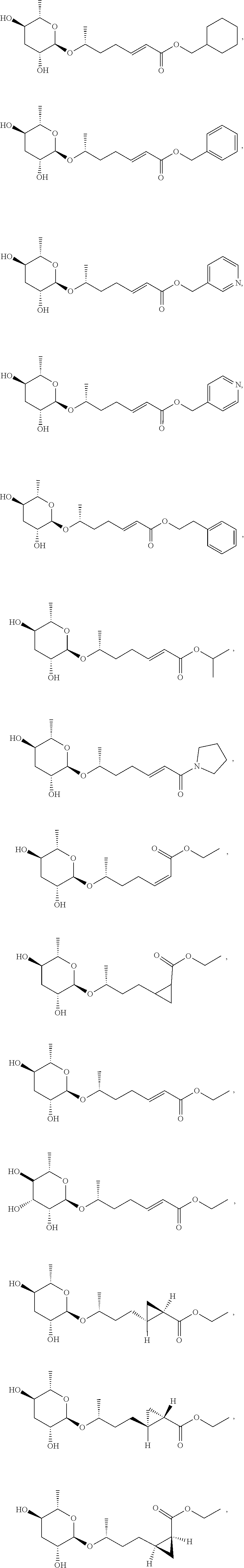

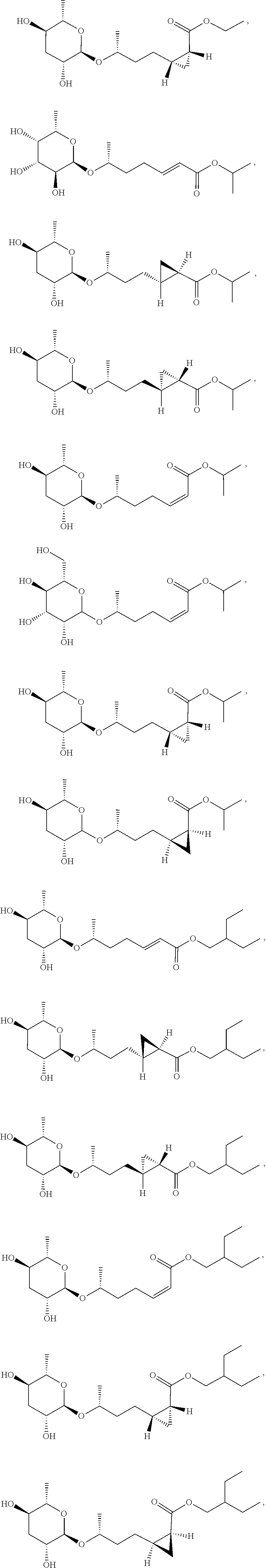

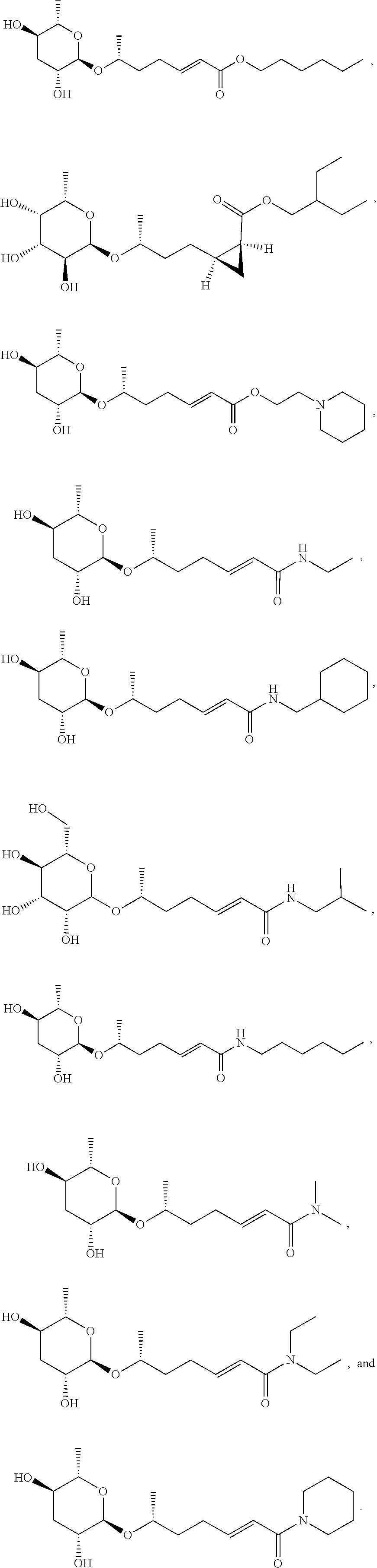

110. The method of claim 108, wherein the compound is selected from

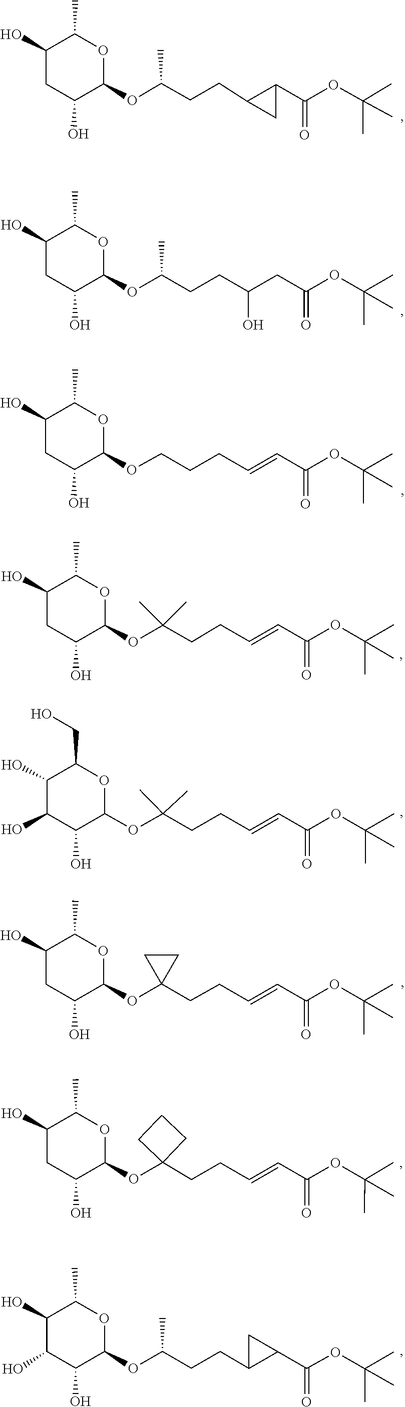

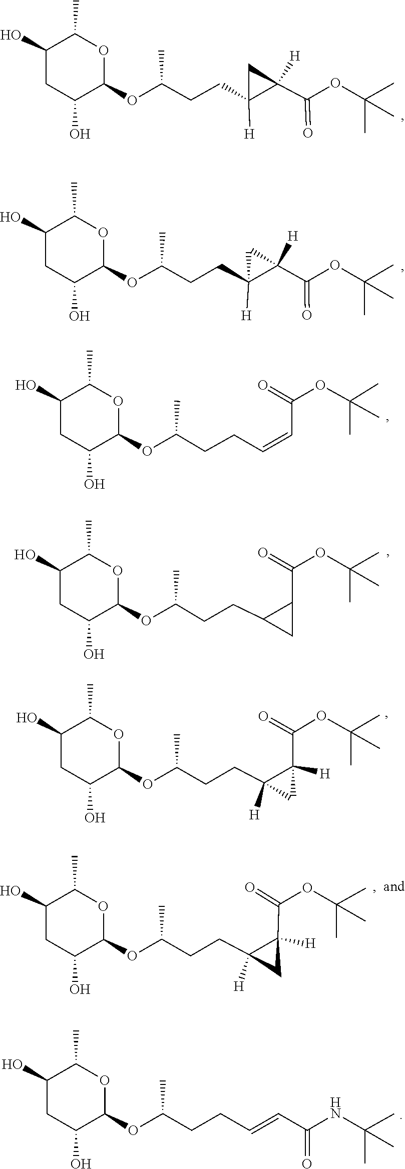

111. The method of claim 102, wherein the compound has the structure of Formula IV: 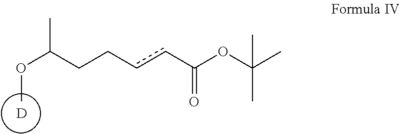
wherein
Ring D is selected from: 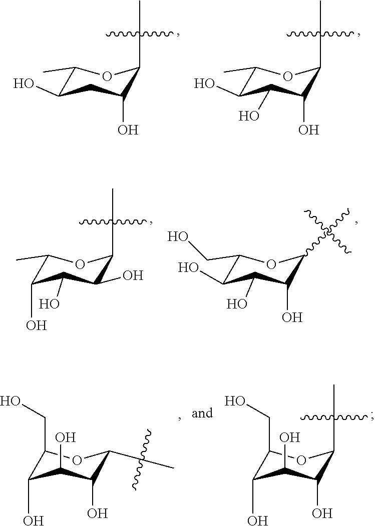
represents the point of attachment of Ring D to the oxygen atom; and [symbol]
is a double bond or a single bond.

112. The method of claim 111, wherein the compound has the structure:

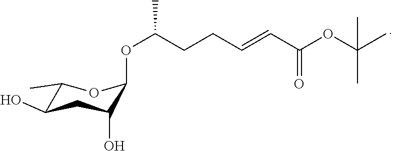

113. The method of claim 111, wherein the compound has the structure:

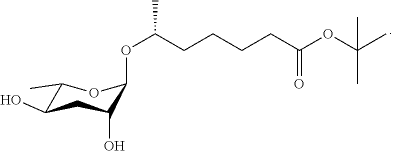

114. The method of claim 111, wherein the compound is selected from

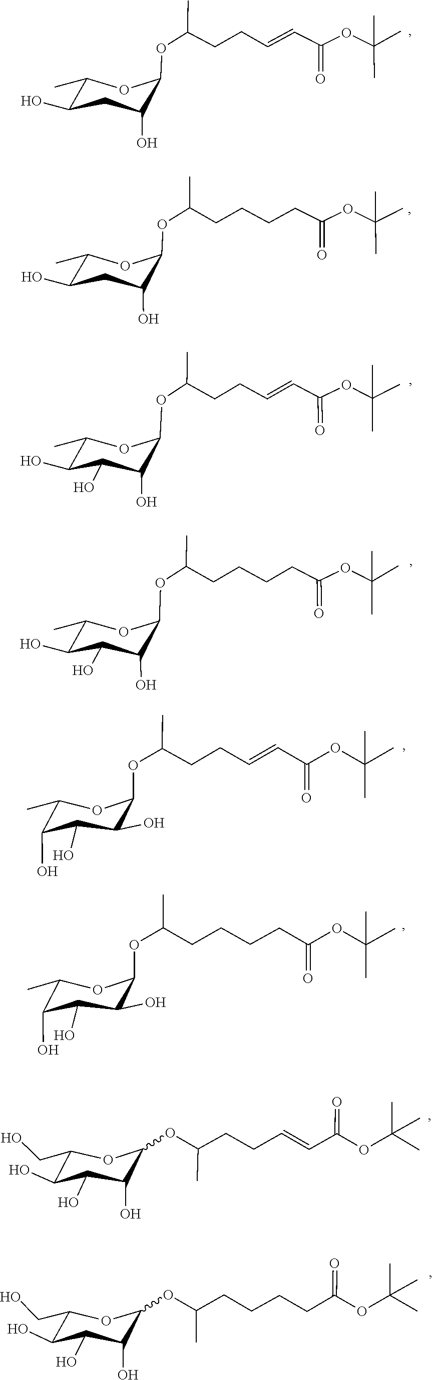

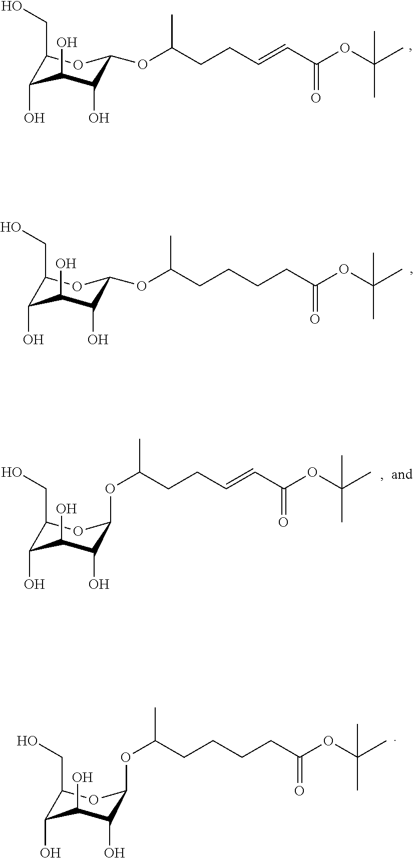

115. The method of claim 102, wherein administering comprises orally administering.

116. The method of claim 102, wherein administering comprises administering into the subject's gastrointestinal tract.

117. The method of claim 102, wherein administering comprises one or more of: oral, intravenous, intraperitoneal and intramuscular administration.

118. The method of claim 102, wherein administering comprises administering a suppository comprising ascaroside.

119. The method of claim 102, wherein administering comprises administering a time-release formulation of ascaroside.

120. The method of claim 102, wherein administering comprises administering multiple doses of ascaroside daily.

121. The method of claim 102, wherein modifying the composition of the gut microbiota comprises increase the amount of one or more of Bifidobacterium, Akkermansia and Adlercreutzia.

122. The method of claim 102, wherein modifying the composition of the gut microbiota comprises increase the amount of Bifidobacter catenulatum.

123. The method of claim 102, wherein modifying the composition of the gut microbiota comprises increase the diversity of the gut microbiota.

124. The method of claim 102, wherein modifying the composition of the gut microbiota comprises increasing the diversity of the gut microbiota and increasing the amount of one or more of: Bifidobacterium, Akkermansia and Adlercreutzia.

125. The method of claim 102, wherein administering comprises administering to the subject during or immediately after a course of antibiotics.

126. A method of treating constipation in a subject, the method comprising administering to the subject's gastrointestinal system a composition comprising pharmaceutically-acceptable carrier and an effective amount of ascaroside derivative to increase fecal softness, wherein the ascaroside derivative has the structure of Formula III: 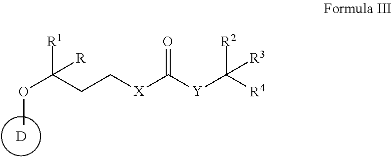
wherein
Ring D is selected from: 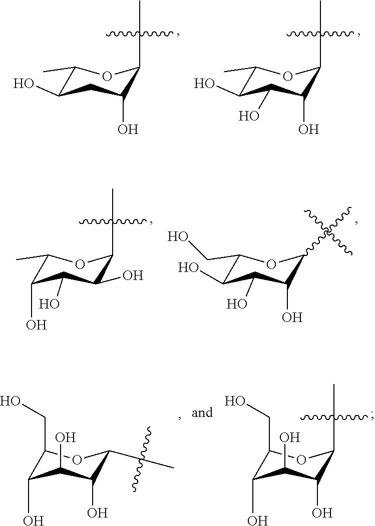
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

127. The method of claim 126, wherein X is —C(R⁵)₂—C(R⁵)₂—.

128. The method of claim 126, wherein X is E or Z —CH═CH—.

129. The method of any of claims 126-128, wherein Y is NR⁶.

130. The method of any of claims 126-128, wherein Y is O.

131. The method of any of claims 126-130, wherein
R² is H;
R³ is H or CH₃; and
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl, or, when Y is NR⁶, R⁴ and NR⁶, taken together with the carbon atom to which they are bonded, form a 5- or 6-membered heterocyclic ring.

132. The method of any of claims 126-130, wherein
R², R³ and R⁴ are each CH₃.

133. The method of any of claims 126-132, wherein the compound is selected from

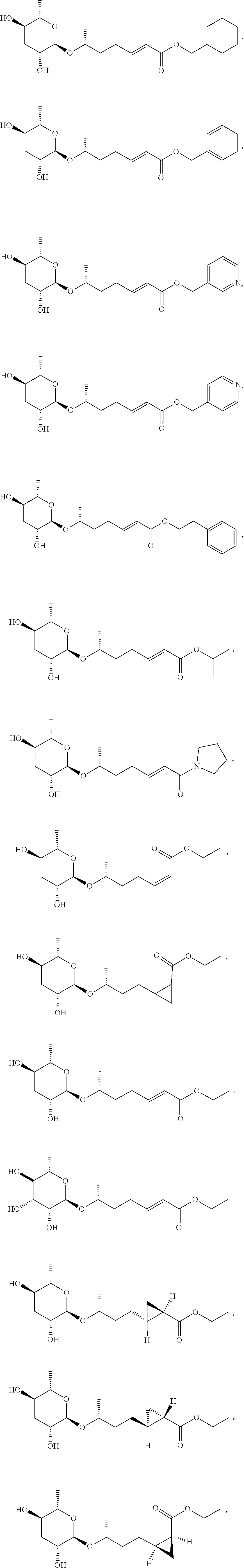

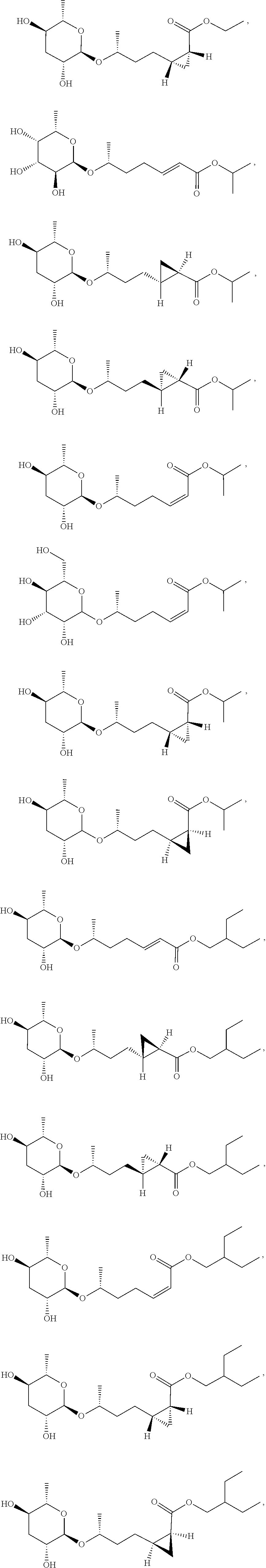

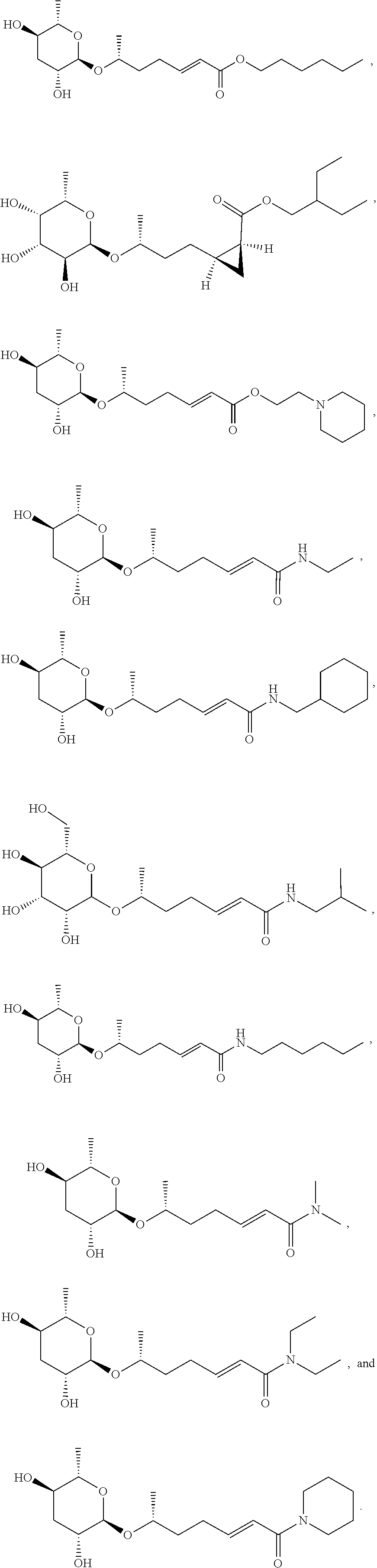

134. The method of claim 132, wherein the compound is selected from

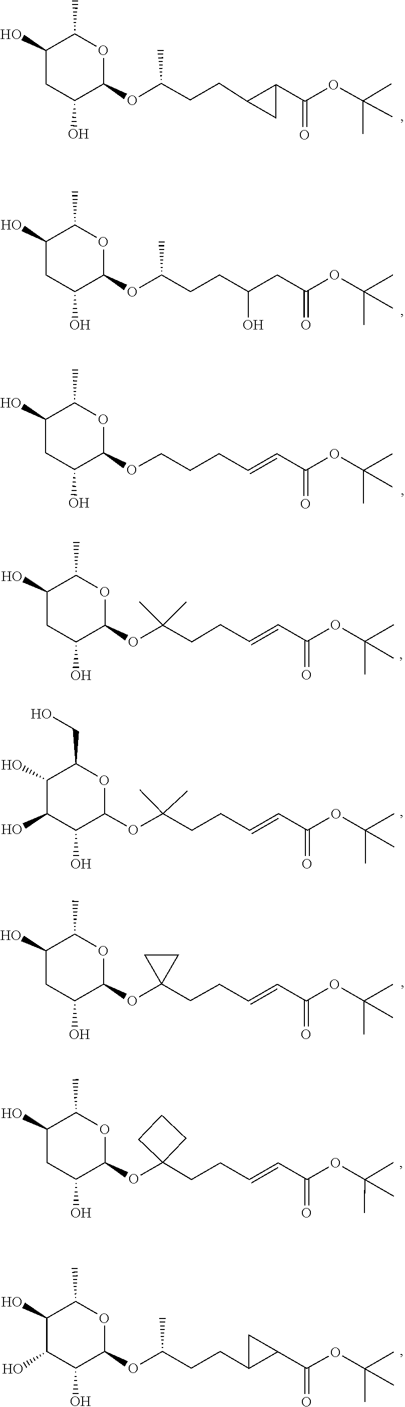

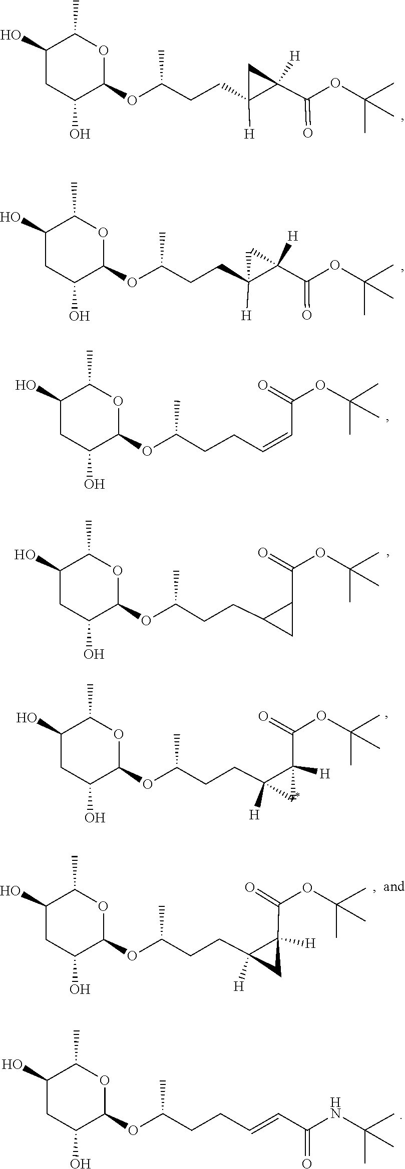

135. The method of claim 126, wherein the compound has the structure of Formula IV: 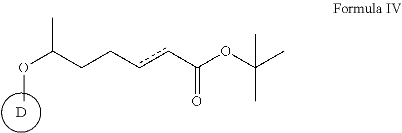
wherein
Ring D is selected from: 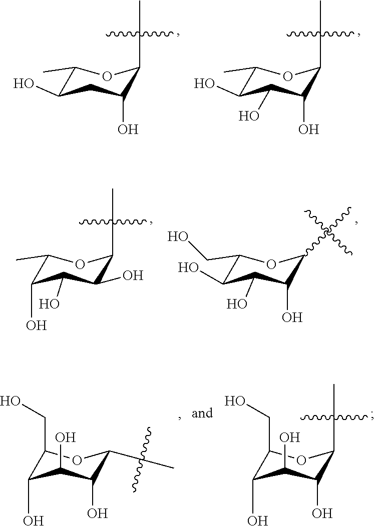
represents the point of attachment of Ring D to the oxygen atom; and [symbol]
is a double bond or a single bond.

136. The method of claim 135, wherein the compound has the structure:

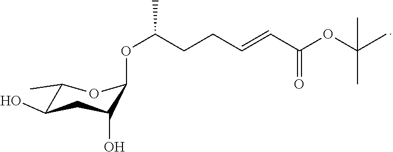

137. The method of claim 135, wherein the compound has the structure:

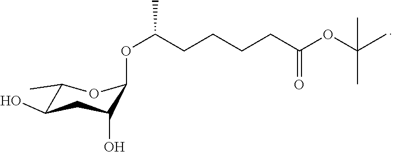

138. The method of claim 135, wherein the compound is selected from

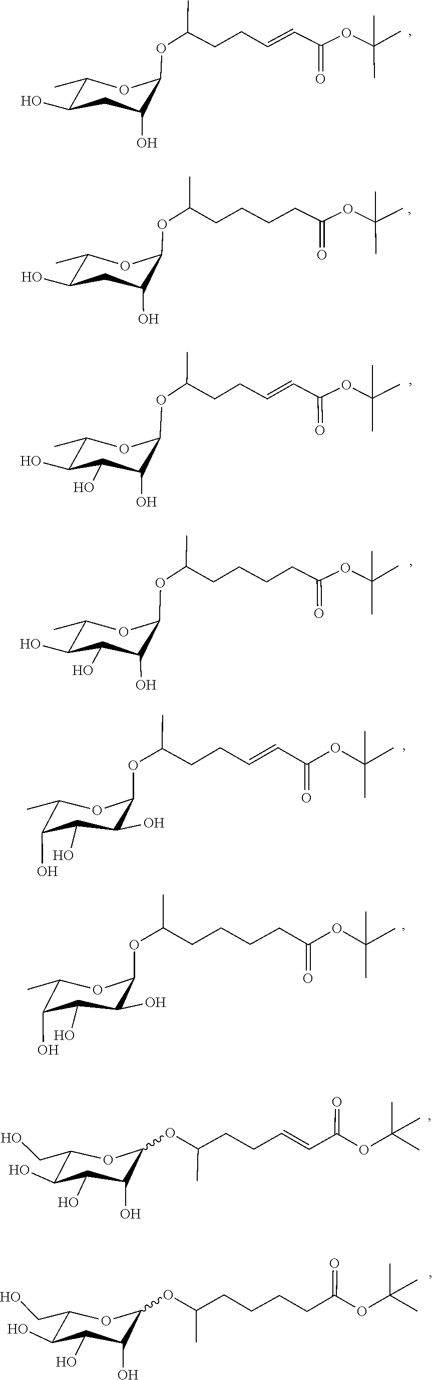

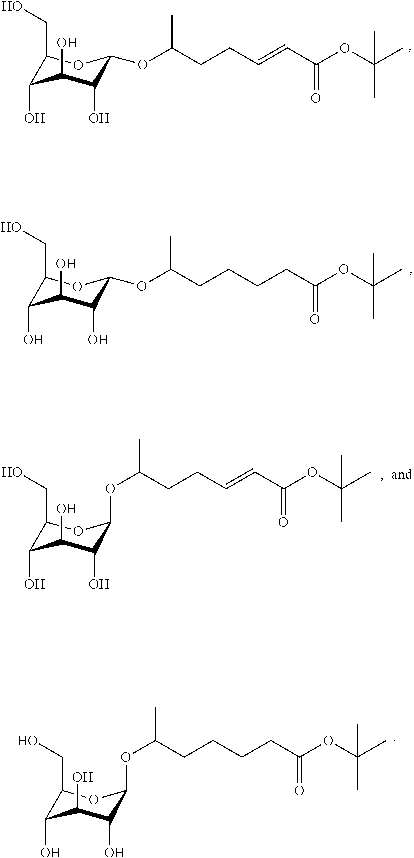

139. The method of claim 126, wherein administering comprises orally administering.

140. The method of claim 126, wherein administering comprises administering into the subject's gastrointestinal tract.

141. The method of claim 126, wherein administering comprises one or more of: oral, intravenous, intraperitoneal and intramuscular administration.

142. The method of claim 126, wherein administering comprises administering a suppository.

143. The method of claim 126, wherein administering comprises administering a time-release formulation of ascaroside.

144. The method of claim 126, wherein administering comprises administering multiple doses of ascaroside daily.

145. The method of claim 126, wherein increasing fecal softness comprises increase the amount of one or more of Bifidobacterium, Akkermansia and Adlercreutzia.

146. The method of claim 126, wherein increasing fecal softness comprises increase the amount of Bifidobacter catenulatum.

147. The method of claim 126, wherein increasing fecal softness comprises increase the diversity of the gut microbiota.

148. The method of claim 126, wherein increasing fecal softness comprises increasing the diversity of the gut microbiota and increasing the amount of one or more of: Bifidobacterium, Akkermansia and Adlercreutzia.

149. A composition of an ascaroside derivative for increasing the diversity of a subject's gut microbiota and/or treating constipation, the composition comprising a pharmaceutically acceptable carrier and a compound having the structure of Formula III: 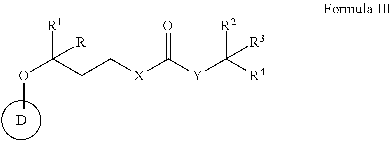
wherein Ring D is selected from 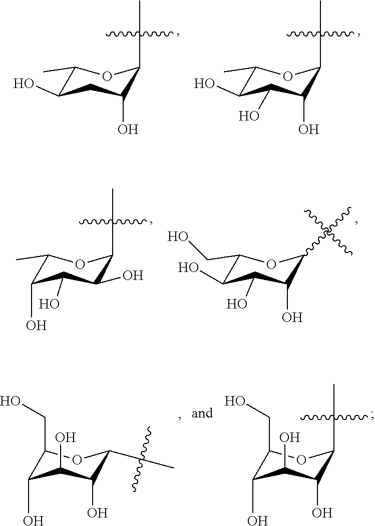
represents the point of attachment of Ring D to the oxygen atom;
X is —C(R⁵)₂—C(R⁵)₂— or E or Z —CH═CH—;
Y is O or NR⁶;
R is H or CH₃,
R¹ is H or CH₃;
or, R and R¹, taken together with the carbon atom to which they are bonded, form a 3- or 4-membered carbocyclic ring;
R² is H or CH₃;
R³ is H or CH₃;
R⁴ is CH₃, CH₂CH₃, a straight- or branched-chain C₃-C₆ alkyl, a C₅-C₇ cycloalkyl, a 6-membered aryl or heteroaryl, an aryl-substituted C₁-C₆ alkyl, or a heterocyclyl-substituted C₁-C₆ alkyl;
R⁵, independently for each occurrence, is H or OH,
or two instances of R⁵, taken together with the carbon atom or carbon atoms to which they are bonded, form a 3-membered carbocyclic ring; and
R⁶ is H or C₁-C₆ alkyl,
or, when Y is NR⁶, R⁴ and NR⁶, taken together with the carbon atom to which they are bonded, form a 5- or 6-membered heterocyclic ring.

150. The composition of claim 149, wherein the compound is selected from

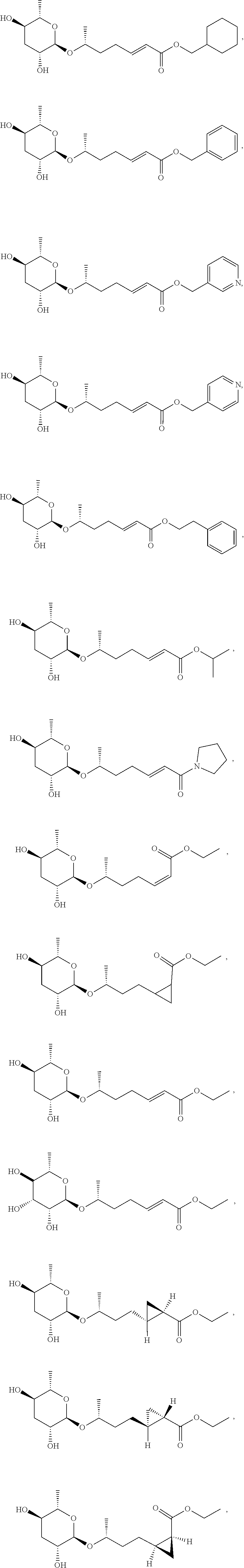

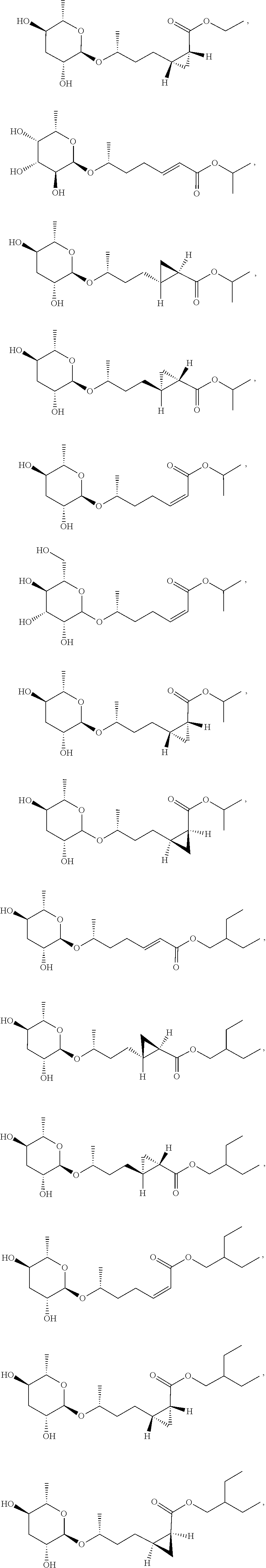

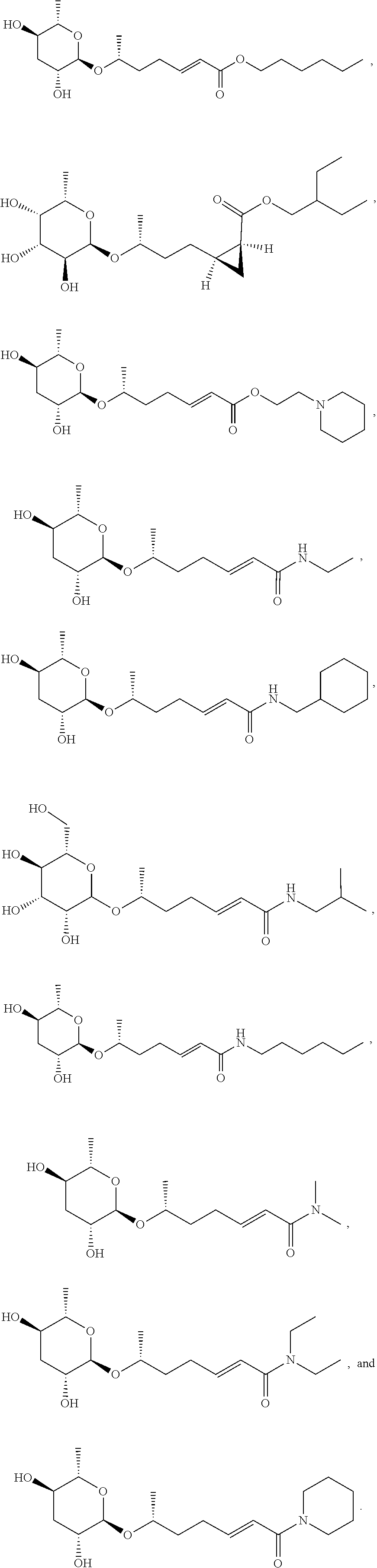

151. The composition of claim 149, wherein the compound is selected from

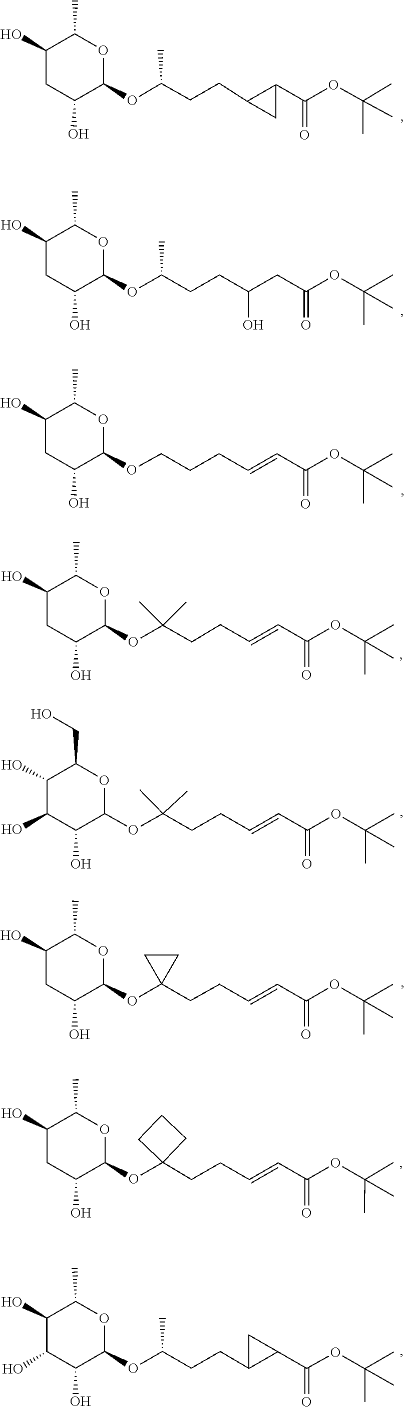

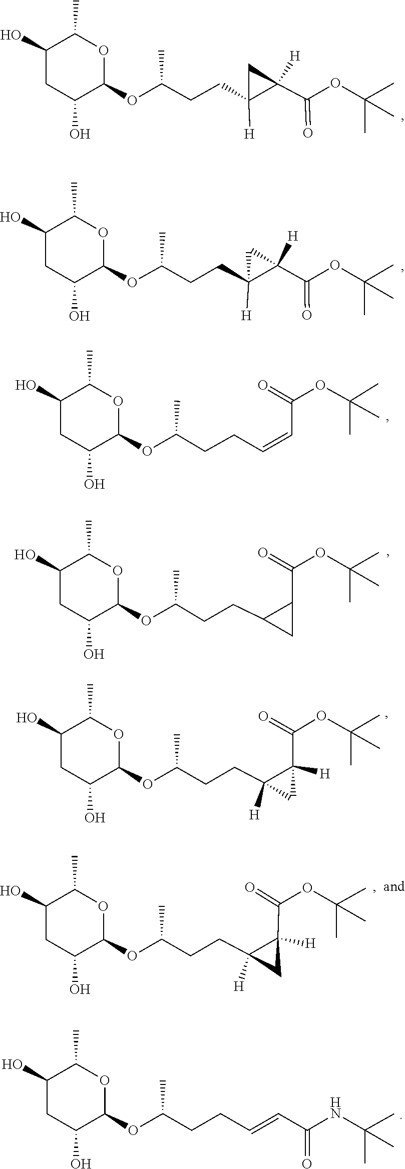

152. The composition of claim 149, wherein the compound has the structure of Formula IV: 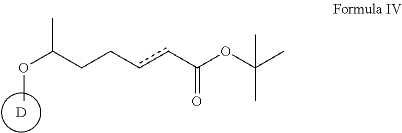
wherein Ring D is selected from 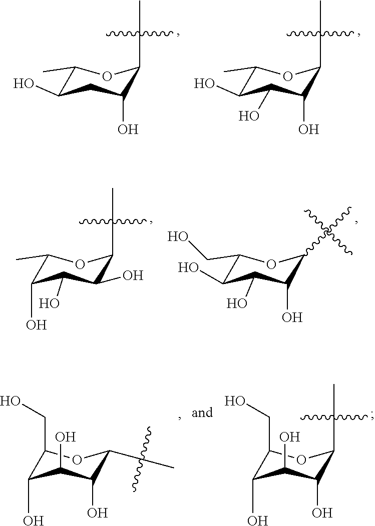
represents the point of attachment of Ring D to the oxygen atom; and [symbol]
is a double bond or a single bond.

153. The composition of claim 149, wherein the compound has the structure:

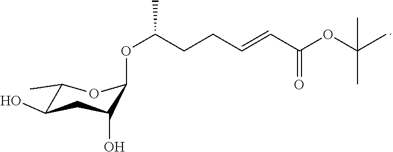

154. The composition of claim 149, wherein the compound has the structure:

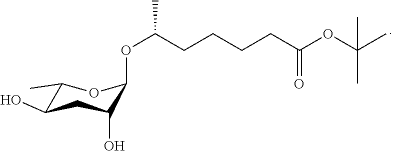

155. The composition of claim 149, wherein the compound is selected from 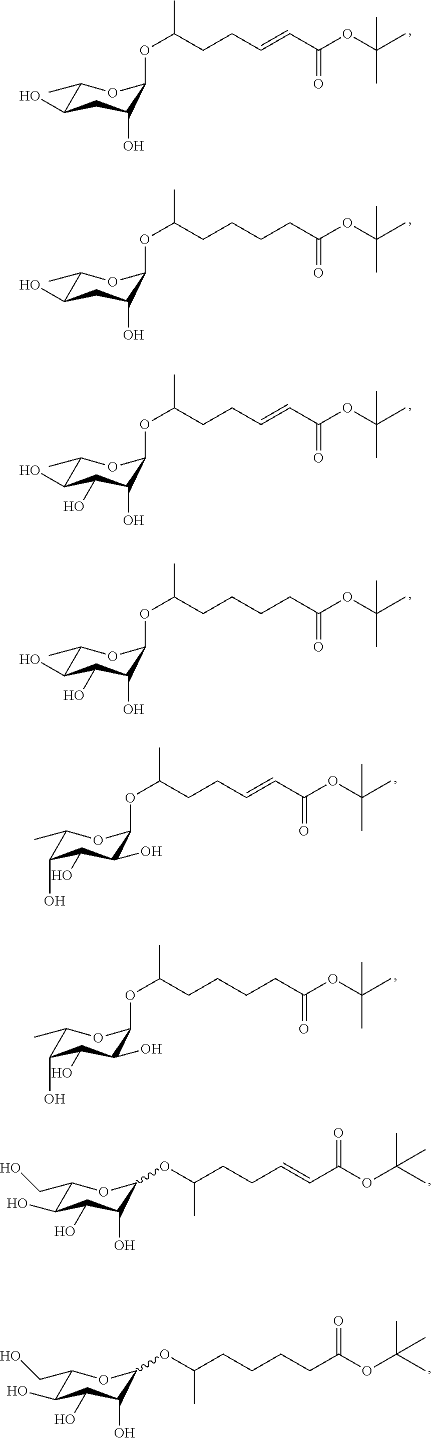 [chemical structure]
